(19) United States
(12) Patent Application Publication
(10) Pub. No.: US 2018/0258064 A1
(43) Pub. Date: Sep. 13, 2018

(54) SOLID FORMS OF 3-(5-AMINO-2-METHYL-4-OXO-4H-QUINAZOLIN-3-YL)-PIPERIDINE-2,6-DIONE, AND THEIR PHARMACEUTICAL COMPOSITIONS AND USES

(71) Applicant: Celgene Corporation, Summit, NJ (US)

(72) Inventors: Lianfeng Huang, Basking Ridge, NJ (US); Daozhong Zou, Raritan, NJ (US)

(21) Appl. No.: 15/913,774

(22) Filed: Mar. 6, 2018

Related U.S. Application Data
(60) Provisional application No. 62/468,264, filed on Mar. 7, 2017.

(51) Int. Cl. C07D 401/04; A61K 31/517

(52) U.S. Cl. CPC C07D 401/04; C07B 2200/13; A61K 31/517

(57) ABSTRACT

Solid forms comprising 3-(5-amino-2-methyl-4-oxo-4H-quinazolin-3-yl)-piperidine-2,6-dione, compositions comprising the solid forms, methods of making the solid forms and methods of their uses are disclosed.

### 1. FIELD

This application claims priority of U.S. Provisional Application No. 62/468,264 filed Mar. 7, 2017, the disclosure of which is incorporated herein by reference in its entirety.

### 2. FIELD

Provided herein are solid forms comprising 3-(5-amino-2-methyl-4-oxo-4H-quinazolin-3-yl)-piperidine-2,6-dione or salts thereof, pharmaceutical compositions thereof, and such solid forms for use in methods for the treatment of diseases or disorders.

### 3. BACKGROUND OF THE DISCLOSURE

The preparation and selection of a solid form of a pharmaceutical compound is complex, given that a change in solid form may affect a variety of physical and chemical properties, which may provide benefits or drawbacks in processing, formulation, stability and bioavailability, among other important pharmaceutical characteristics. Potential pharmaceutical solids include crystalline solids and amorphous solids. Amorphous solids are characterized by a lack of long-range structural order, whereas crystalline solids are characterized by structural periodicity. The desired class of pharmaceutical solid depends upon the specific application; amorphous solids are sometimes selected on the basis of, e.g., an enhanced dissolution profile, while crystalline solids may be desirable for properties such as, e.g., physical or chemical stability (see, e.g., S. R. Vippagunta et al., Adv. Drug. Deliv. Rev., (2001) 48:3-26; L. Yu, Adv. Drug. Deliv. Rev., (2001) 48:27-42).

Whether crystalline or amorphous, potential solid forms of a pharmaceutical compound include single-component and multiple-component solids. Single-component solids consist essentially of the pharmaceutical compound in the absence of other compounds. Variety among single-component crystalline materials may potentially arise, e.g., from the phenomenon of polymorphism, wherein multiple three-dimensional arrangements exist for a particular pharmaceutical compound (see, e.g., S. R. Byrn et al., Solid State Chemistry of Drugs, (1999) SSCI, West Lafayette). The importance of studying polymorphs was underscored by the case of Ritonavir, an HIV protease inhibitor that was formulated as soft gelatin capsules. About two years after the product was launched, the unanticipated precipitation of a new, less soluble polymorph in the formulation necessitated the withdrawal of the product from the market until a more consistent formulation could be developed (see S. R. Chemburkar et al., Org. Process Res. Dev., (2000) 4:413-417).

Additional diversity among the potential solid forms of a pharmaceutical compound may arise, e.g., from the possibility of multiple-component solids. Crystalline solids comprising two or more ionic species may be termed salts (see, e.g., Handbook of Pharmaceutical Salts: Properties, Selection and Use, P. H. Stahl and C. G. Wermuth, Eds., (2002), Wiley, Weinheim). Additional types of multiple-component solids that may potentially offer other property improvements for a pharmaceutical compound or salt thereof include, e.g., hydrates, solvates, cocrystals and clathrates, among others (see, e.g., S. R. Byrn et al., Solid State Chemistry of Drugs, (1999) SSCI, West Lafayette). Moreover, multiple-component crystal forms may potentially be susceptible to polymorphism, wherein a given multiple-component composition may exist in more than one three-dimensional crystalline arrangement. The preparation of solid forms is of great importance in the development of a safe, effective, stable and marketable pharmaceutical compound.

Provided herein are embodiments addressing a need for solid forms of 3-(5-amino-2-methyl-4-oxo-4H-quinazolin-3-yl)-piperidine-2,6-dione (“Compound 1”). Compound 1 was described in U.S. Pat. No. 7,635,700, the disclosure of which is incorporated herein by reference in its entirety.

### 4. SUMMARY

Provided herein are solid forms comprising 3-(5-amino-2-methyl-4-oxo-4H-quinazolin-3-yl)-piperidine-2,6-dione (Compound 1) or a stereoisomer thereof, or a pharmaceutically acceptable salt, solvate, hydrate, cocrystal, clathrate, or polymorph thereof.

In certain embodiments, the solid forms provided herein are single-component or multiple-component crystal forms, including, but not limited to, salts, cocrystals and/or solvates, comprising Compound 1. In one embodiment, provided herein are solid forms of salts of Compound 1. Without intending to be limited by any particular theory, the storage stability, compressibility, bulk density or dissolution properties of certain solid forms described herein are believed to be beneficial for manufacturing, formulation and bioavailability of Compound 1 or salts or cocrystals thereof.

In certain embodiments, solid forms provided herein include solid forms comprising salts of Compound 1, such as sulfate salts (sulfuric acid salt), mesylate salts (methanesulfonate salt), esylate salts (ethanesulfonate salt), besylate salts (benzenesulfonate salt), napadisylate salts (naphthalene-1,5-disulfonic acid salt), napsilate salts (naphthalene-2-sulfonic acid salt), thiocyanate salts (thiocyanic acid salt) and tosylate salts (p-toluenesulfonate salt).

In certain embodiments, solid forms provided herein include cocrystals comprising Compound 1, such as acetic acid cocrystal comprising Compound 1, isethionic acid cocrystal comprising Compound 1, lauric acid cocrystal comprising Compound 1, maleic acid cocrystal comprising Compound 1 and phosphoric acid cocrystal comprising Compound 1.

Further provided herein are pharmaceutical compositions, single unit dosage forms, dosing regimens, and kits, which comprise a solid form of a pharmaceutically acceptable salt of Compound 1 or a cocrystal comprising Compound 1; and a pharmaceutically acceptable carrier.

Provided herein is a solid form of a pharmaceutically acceptable salt of Compound 1 or a cocrystal comprising Compound 1 that can be used in the methods provided herein.

Also provided herein are methods of treating diseases and disorders utilizing a solid form of a pharmaceutically acceptable salt of Compound 1 or a cocrystal comprising Compound 1. The solid form of a pharmaceutically acceptable salt of Compound 1 or a cocrystal comprising Compound 1 provided herein can be used in methods of treating diseases and disorders.

Additionally provided herein are methods of treating and managing various diseases or disorders, which comprise administering to a patient a therapeutically effective amount of a solid form of a pharmaceutically acceptable salt of Compound 1 or a cocrystal comprising Compound 1. The solid form of a pharmaceutically acceptable salt of Compound 1 or a cocrystal comprising Compound 1 provided herein can be used in methods of treating and managing various diseases or disorders, which comprise administering to a patient a therapeutically effective amount of said solid form of a pharmaceutically acceptable salt of Compound 1 or said cocrystal comprising Compound 1.

In certain embodiments, provided herein are methods of making, isolating and/or characterizing the solid forms provided herein.

The solid forms provided herein are useful as active pharmaceutical ingredients for the preparation of formulations for use in patients. Thus, embodiments herein encompass the use of these solid forms as a final drug product. Certain embodiments provide solid forms useful in making final dosage forms with improved properties, e.g., powder flow properties, compaction properties, tableting properties, stability properties, and excipient compatibility properties, among others, that are needed for manufacturing, processing, formulation and/or storage of final drug products. Certain embodiments herein provide pharmaceutical compositions comprising a single-component crystal form, a multiple-component crystal form, a single-component amorphous form and/or a multiple-component amorphous form comprising 3-(5-amino-2-methyl-4-oxo-4H-quinazolin-3-yl)-piperidine-2,6-dione and a pharmaceutically acceptable diluent, excipient or carrier. The solid forms and the final drug products provided herein are useful, for example, for the treatment, prevention or management of diseases and disorders provided herein.

### 4.1. BRIEF DESCRIPTION OF THE FIGURES

FIG. 1A provides an X-ray Powder Diffraction (“XRPD”) pattern of Form A of besylate salt of 3-(5-amino-2-methyl-4-oxo-4H-quinazolin-3-yl)-piperidine-2,6-dione.

FIG. 1B provides a post-DVS plot for X-ray Powder Diffraction (“XRPD”) pattern of Form A of besylate salt of 3-(5-amino-2-methyl-4-oxo-4H-quinazolin-3-yl)-piperidine-2,6-dione.

FIG. 2 provides Differential Scanning Calorimetry (“DSC”) and Thermal Gravimetric Analysis (“TGA”) plots of Form A of besylate salt of 3-(5-amino-2-methyl-4-oxo-4H-quinazolin-3-yl)-piperidine-2,6-dione.

FIG. 3 provides a Dynamic Vapor Sorption (DVS) isotherm plot of Form A of besylate salt of 3-(5-amino-2-methyl-4-oxo-4H-quinazolin-3-yl)-piperidine-2,6-dione.

FIG. 4A, FIG. 4B and FIG. 4C show a representative 1H NMR spectrum of Form A of besylate salt of 3-(5-amino-2-methyl-4-oxo-4H-quinazolin-3-yl)-piperidine-2,6-dione.

FIG. 5 provides an X-ray Powder Diffraction (“XRPD”) pattern of Form B of esylate salt of 3-(5-amino-2-methyl-4-oxo-4H-quinazolin-3-yl)-piperidine-2,6-dione.

FIG. 6 provides an overlay of X-ray Powder Diffraction (“XRPD”) patterns of Form A and Form B of esylate salt of 3-(5-amino-2-methyl-4-oxo-4H-quinazolin-3-yl)-piperidine-2,6-dione.

FIG. 7 provides a Differential Scanning Calorimetry (“DSC”) and Thermal Gravimetric Analysis (“TGA”) plots of Form B of esylate salt of 3-(5-amino-2-methyl-4-oxo-4H-quinazolin-3-yl)-piperidine-2,6-dione.

FIG. 8A and FIG. 8B show a representative 1H NMR spectrum of Form B of esylate salt of 3-(5-amino-2-methyl-4-oxo-4H-quinazolin-3-yl)-piperidine-2,6-dione.

FIG. 9 provides an X-ray Powder Diffraction (“XRPD”) pattern of Form A of napadisylate salt of 3-(5-amino-2-methyl-4-oxo-4H-quinazolin-3-yl)-piperidine-2,6-dione.

FIG. 10 provides Differential Scanning Calorimetry (“DSC”) and Thermal Gravimetric Analysis (“TGA”) plots of Form A of napadisylate salt of 3-(5-amino-2-methyl-4-oxo-4H-quinazolin-3-yl)-piperidine-2,6-dione.

FIG. 11A and FIG. 11B show a representative 1H NMR spectrum of Form A of napadisylate salt of 3-(5-amino-2-methyl-4-oxo-4H-quinazolin-3-yl)-piperidine-2,6-dione.

FIG. 12 provides an X-ray Powder Diffraction (“XRPD”) pattern of Form A of napsilate salt of 3-(5-amino-2-methyl-4-oxo-4H-quinazolin-3-yl)-piperidine-2,6-dione.

FIG. 13 provides Differential Scanning Calorimetry (“DSC”) and Thermal Gravimetric Analysis (“TGA”) plots of Form A of napsilate salt of 3-(5-amino-2-methyl-4-oxo-4H-quinazolin-3-yl)-piperidine-2,6-dione.

FIG. 14A, FIG. 14B and FIG. 14C show a representative 1H NMR spectrum of Form A of napsilate salt of 3-(5-amino-2-methyl-4-oxo-4H-quinazolin-3-yl)-piperidine-2,6-dione.

FIG. 15 provides an X-ray Powder Diffraction (“XRPD”) pattern of Form A of mesylate salt of 3-(5-amino-2-methyl-4-oxo-4H-quinazolin-3-yl)-piperidine-2,6-dione.

FIG. 16 provides an overlay of X-ray Powder Diffraction (“XRPD”) patterns of Form A and Form B of mesylate salt of 3-(5-amino-2-methyl-4-oxo-4H-quinazolin-3-yl)-piperidine-2,6-dione.

FIG. 17 provides Differential Scanning Calorimetry (“DSC”) and Thermal Gravimetric Analysis (“TGA”) plots of Form A of mesylate salt of 3-(5-amino-2-methyl-4-oxo-4H-quinazolin-3-yl)-piperidine-2,6-dione.

FIG. 18A and FIG. 18B show a representative 1H NMR spectrum of Form A of mesylate salt of 3-(5-amino-2-methyl-4-oxo-4H-quinazolin-3-yl)-piperidine-2,6-dione.

FIG. 19 provides a Dynamic Vapor Sorption (DVS) isotherm plot of Form A of mesylate salt of 3-(5-amino-2-methyl-4-oxo-4H-quinazolin-3-yl)-piperidine-2,6-dione.

FIG. 20 provides an X-ray Powder Diffraction (“XRPD”) pattern of Form B of mesylate salt of 3-(5-amino-2-methyl-4-oxo-4H-quinazolin-3-yl)-piperidine-2,6-dione.

FIG. 21 provides an X-ray Powder Diffraction (“XRPD”) pattern of Form A of sulfate salt of 3-(5-amino-2-methyl-4-oxo-4H-quinazolin-3-yl)-piperidine-2,6-dione.

FIG. 22 provides Differential Scanning Calorimetry (“DSC”) and Thermal Gravimetric Analysis (“TGA”) plots of Form A of sulfate salt of 3-(5-amino-2-methyl-4-oxo-4H-quinazolin-3-yl)-piperidine-2,6-dione.

FIG. 23 provides a Dynamic Vapor Sorption (DVS) isotherm plot of Form A of sulfate salt of 3-(5-amino-2-methyl-4-oxo-4H-quinazolin-3-yl)-piperidine-2,6-dione.

FIG. 24 provides a post-DVS plot for X-ray Powder Diffraction (“XRPD”) pattern of Form A of sulfate salt of 3-(5-amino-2-methyl-4-oxo-4H-quinazolin-3-yl)-piperidine-2,6-dione.

FIG. 25A and FIG. 25B show a representative 1H NMR spectrum of Form A of sulfate salt of 3-(5-amino-2-methyl-4-oxo-4H-quinazolin-3-yl)-piperidine-2,6-dione.

FIG. 26 provides an X-ray Powder Diffraction (“XRPD”) pattern of Form A of thiocyanate salt of 3-(5-amino-2-methyl-4-oxo-4H-quinazolin-3-yl)-piperidine-2,6-dione.

FIG. 27 provides Differential Scanning Calorimetry (“DSC”) and Thermal Gravimetric Analysis (“TGA”) plots of Form A of thiocyanate salt of 3-(5-amino-2-methyl-4-oxo-4H-quinazolin-3-yl)-piperidine-2,6-dione.

FIG. 28 provides an overlay of X-ray Powder Diffraction (“XRPD”) patterns of Form A and Form B of thiocyanate salt of 3-(5-amino-2-methyl-4-oxo-4H-quinazolin-3-yl)-piperidine-2,6-dione.

FIG. 29A and FIG. 29B show a representative 1H NMR spectrum of Form A of thiocyanate salt of 3-(5-amino-2-methyl-4-oxo-4H-quinazolin-3-yl)-piperidine-2,6-dione.

FIG. 30 provides an X-ray Powder Diffraction (“XRPD”) pattern of Form A of tosylate salt of 3-(5-amino-2-methyl-4-oxo-4H-quinazolin-3-yl)-piperidine-2,6-dione.

FIG. 31 provides Differential Scanning Calorimetry (“DSC”) and Thermal Gravimetric Analysis (“TGA”) plots of Form A of tosylate salt of 3-(5-amino-2-methyl-4-oxo-4H-quinazolin-3-yl)-piperidine-2,6-dione.

FIG. 32 provides a Dynamic Vapor Sorption (DVS) isotherm plot of Form A of tosylate salt of 3-(5-amino-2-methyl-4-oxo-4H-quinazolin-3-yl)-piperidine-2,6-dione.

FIG. 33 provides an X-ray Powder Diffraction (“XRPD”) pattern of Form B of tosylate salt of 3-(5-amino-2-methyl-4-oxo-4H-quinazolin-3-yl)-piperidine-2,6-dione.

FIG. 34 provides an overlay of X-ray Powder Diffraction (“XRPD”) patterns of Form A and Form B of tosylate salt of 3-(5-amino-2-methyl-4-oxo-4H-quinazolin-3-yl)-piperidine-2,6-dione.

FIG. 35A and FIG. 35B show a representative 1H NMR spectrum of Form A of tosylate salt of 3-(5-amino-2-methyl-4-oxo-4H-quinazolin-3-yl)-piperidine-2,6-dione.

FIG. 36 provides an X-ray Powder Diffraction (“XRPD”) pattern of 3-(5-amino-2-methyl-4-oxo-4H-quinazolin-3-yl)-piperidine-2,6-dione acetic acid cocrystal Form A.

FIG. 37 provides Differential Scanning Calorimetry (“DSC”) and Thermal Gravimetric Analysis (“TGA”) plots of 3-(5-amino-2-methyl-4-oxo-4H-quinazolin-3-yl)-piperidine-2,6-dione acetic acid cocrystal Form A.

FIG. 38A and FIG. 38B show a representative 1H NMR spectrum of Form A of 3-(5-amino-2-methyl-4-oxo-4H-quinazolin-3-yl)-piperidine-2,6-dione acetic acid cocrystal Form A.

FIG. 39 provides an X-ray Powder Diffraction (“XRPD”) pattern of 3-(5-amino-2-methyl-4-oxo-4H-quinazolin-3-yl)-piperidine-2,6-dione isethionic acid cocrystal Form B.

FIG. 40 provides an overlay of X-ray Powder Diffraction (“XRPD”) patterns of 3-(5-amino-2-methyl-4-oxo-4H-quinazolin-3-yl)-piperidine-2,6-dione isethionic acid cocrystal Form A and Form B.

FIG. 41 provides Differential Scanning Calorimetry (“DSC”) and Thermal Gravimetric Analysis (“TGA”) plots of 3-(5-amino-2-methyl-4-oxo-4H-quinazolin-3-yl)-piperidine-2,6-dione isethionic acid cocrystal Form B.

FIG. 42A and FIG. 42B show a representative 1H NMR spectrum of 3-(5-amino-2-methyl-4-oxo-4H-quinazolin-3-yl)-piperidine-2,6-dione isethionic acid cocrystal Form B.

FIG. 43 provides an X-ray Powder Diffraction (“XRPD”) pattern of 3-(5-amino-2-methyl-4-oxo-4H-quinazolin-3-yl)-piperidine-2,6-dione lauric acid cocrystal Form A.

FIG. 44 provides Differential Scanning Calorimetry (“DSC”) and Thermal Gravimetric Analysis (“TGA”) plots of 3-(5-amino-2-methyl-4-oxo-4H-quinazolin-3-yl)-piperidine-2,6-dione lauric acid cocrystal Form A.

FIG. 45A and FIG. 45B show a representative 1H NMR spectrum of Form A of 3-(5-amino-2-methyl-4-oxo-4H-quinazolin-3-yl)-piperidine-2,6-dione lauric acid cocrystal Form A.

FIG. 46 provides an X-ray Powder Diffraction (“XRPD”) pattern of 3-(5-amino-2-methyl-4-oxo-4H-quinazolin-3-yl)-piperidine-2,6-dione maleic acid cocrystal Form A.

FIG. 47 provides Differential Scanning Calorimetry (“DSC”) and Thermal Gravimetric Analysis (“TGA”) plots of 3-(5-amino-2-methyl-4-oxo-4H-quinazolin-3-yl)-piperidine-2,6-dione maleic acid cocrystal Form A.

FIG. 48A and FIG. 48B show a representative 1H NMR spectrum of Form A of 3-(5-amino-2-methyl-4-oxo-4H-quinazolin-3-yl)-piperidine-2,6-dione maleic acid cocrystal Form A.

FIG. 49 provides an X-ray Powder Diffraction (“XRPD”) pattern of 3-(5-amino-2-methyl-4-oxo-4H-quinazolin-3-yl)-piperidine-2,6-dione phosphoric acid cocrystal Form A.

FIG. 50 provides Differential Scanning Calorimetry (“DSC”) and Thermal Gravimetric Analysis (“TGA”) plots of 3-(5-amino-2-methyl-4-oxo-4H-quinazolin-3-yl)-piperidine-2,6-dione phosphoric acid cocrystal Form A.

FIG. 51A and FIG. 51B show a representative 1H NMR spectrum of Form A of 3-(5-amino-2-methyl-4-oxo-4H-quinazolin-3-yl)-piperidine-2,6-dione phosphoric acid cocrystal Form A.

### 4.2. DEFINITIONS

The use of the word “a” or “an” when used in conjunction with the term “comprising” in the claims and/or the specification can mean “one,” but it is also consistent with the meaning of “one or more,” “at least one,” and “one or more than one.”

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein, term “Compound 1” refers to 3-(5-amino-2-methyl-4-oxo-4H-quinazolin-3-yl)-piperidine-2,6-dione. The ¹H NMR spectrum of Compound 1 is substantially as follows: δ (DMSO-d₆): 2.10-2.17 (m, 1H), 2.53 (s, 3H), 2.59-2.69 (m, 2H), 2.76-2.89 (m, 1H), 5.14 (dd, J=6, 11 Hz, 1H), 6.56 (d, J=8 Hz, 1H), 6.59 (d, J=8 Hz, 1H), 7.02 (s, 2H), 7.36 (t, J=8 Hz, 1H), 10.98 (s, 1H). The ¹³C NMR spectrum of Compound 1 is substantially as follows: δ (DMSO-d₆): 20.98, 23.14, 30.52, 55.92, 104.15, 110.48, 111.37, 134.92, 148.17, 150.55, 153.62, 162.59, 169.65, 172.57.

Compound 1 is 3-(5-amino-2-methyl-4-oxo-4H-quinazolin-3-yl)-piperidine-2,6-dione, which has the following structure:

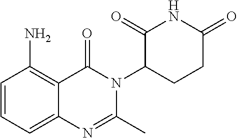 [SMILES: Cc1nc2cccc(N)c2c(=O)n1C1CCC(=O)NC1=O]

As used herein, the term “patient” refers to a mammal, particularly a human.

As used herein, the term “pharmaceutically acceptable salt” refers to a salt prepared from a pharmaceutically acceptable non-toxic acid or base, including inorganic acids and bases and organic acids and bases.

As used herein, term “adverse effects” includes, but is not limited to gastrointestinal, renal and hepatic toxicities, leukopenia, increases in bleeding times, thrombocytopenia, prolongation of gestation, nausea, vomiting, somnolence, asthenia, dizziness, teratogenicity, extra-pyramidal symptoms, akathisia, cardiotoxicity including cardiovascular disturbances, inflammation, male sexual dysfunction, and elevated serum liver enzyme levels. The term “gastrointestinal toxicities” includes, but is not limited to, nausea, vomiting, gastric and intestinal ulcerations and erosions. The term “renal toxicities” includes, but is not limited to, such conditions as papillary necrosis and chronic interstitial nephritis.

As used herein and unless otherwise indicated, the phrases “reduce or avoid adverse effects” and “reducing or avoiding adverse effects” mean the reduction of the severity of one or more adverse effects as defined herein.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

As used herein and unless otherwise specified, the terms “solid form” and related terms refer to a physical form which is not predominantly in a liquid or a gaseous state. As used herein and unless otherwise specified, the term “solid form” and related terms, refer to a physical form comprising Compound 1, a salt or a cocrystal thereof, which is not predominantly in a liquid or a gaseous state. Solid forms may be crystalline, amorphous, or mixtures thereof. A “multiple-component” solid form comprising Compound 1 comprises a significant quantity of one or more additional species, such as ions and/or molecules, within the solid form. In certain embodiments, a “multiple-component” solid form comprising Compound 1 comprises a salt of Compound 1. For example, in particular embodiments, a crystalline multiple-component solid form comprising Compound 1 further comprises one or more species non-covalently bonded at regular positions in the crystal lattice. Multiple-component solid forms comprising Compound 1 include cocrystals, solvates (e.g., hydrates), and clathrates of Compound 1. In particular embodiments, the term “solid form comprising Compound 1” and related terms include single-component and multiple-component solid forms comprising Compound 1. In particular embodiments, “solid forms comprising Compound 1” and related terms include crystal forms comprising Compound 1, amorphous forms comprising Compound 1, and mixtures thereof.

As used herein and unless otherwise specified, the term “crystalline” and related terms used herein, when used to describe a compound, substance, modification, material, component or product, unless otherwise specified, mean that the compound, substance, modification, material, component or product is substantially crystalline as determined by X-ray diffraction. See, e.g., Remington: The Science and Practice of Pharmacy, 21^{st} edition, Lippincott, Williams and Wilkins, Baltimore, Md. (2005); The United States Pharmacopeia, 23^{rd} ed., 1843-1844 (1995).

As used herein and unless otherwise specified, the term “crystal forms,” “crystalline forms” and related terms herein refer to solid forms that are crystalline. Crystal forms include single-component crystal forms and multiple-component crystal forms, and include, but are not limited to, salts (e.g., a hydrochloride salt), polymorphs, solvates, hydrates, and/or other molecular complexes. In certain embodiments, a crystal form of a substance may be substantially free of amorphous forms and/or other crystal forms. In certain embodiments, a crystal form of a substance may contain less than about 1%, 2%, 3%, 4%, 5%, 6%, 7%, 8%, 9%, 10%, 15%, 20%, 25%, 30%, 35%, 40%, 45% or 50% of one or more amorphous forms and/or other crystal forms on a weight basis. In certain embodiments, a crystal form of a substance may be physically and/or chemically pure. In certain embodiments, a crystal form of a substance may be about 99%, 98%, 97%, 96%, 95%, 94%, 93%, 92%, 91% or 90% physically and/or chemically pure.

As used herein and unless otherwise specified, the terms “polymorphs,” “polymorphic forms” and related terms herein, refer to two or more crystal forms that consist essentially of the same molecule, molecules, and/or ions. Like different crystal forms, different polymorphs may have different physical properties such as, e.g., melting temperature, heat of fusion, solubility, dissolution properties and/or vibrational spectra, as a result of the arrangement or conformation of the molecules and/or ions in the crystal lattice. The differences in physical properties may affect pharmaceutical parameters such as storage stability, compressibility and density (important in formulation and product manufacturing), and dissolution rate (an important factor in bioavailability). Differences in stability can result from changes in chemical reactivity (e.g., differential oxidation, such that a dosage form discolors more rapidly when comprised of one polymorph than when comprised of another polymorph) or mechanical changes (e.g., tablets crumble on storage as a kinetically favored polymorph converts to thermodynamically more stable polymorph) or both (e.g., tablets of one polymorph are more susceptible to breakdown at high humidity). As a result of solubility/dissolution differences, in the extreme case, some solid-state transitions may result in lack of potency or, at the other extreme, toxicity. In addition, the physical properties may be important in processing (e.g., one polymorph might be more likely to form solvates or might be difficult to filter and wash free of impurities, and particle shape and size distribution might be different between polymorphs).

Unless otherwise specified, the term “cocrystal” or “co-crystal,” as used herein, refers to a crystalline material comprised of two or more non-volative compounds bound together in a crystal lattice by non-covalent interactions.

Unless otherwise specified, the term “pharmaceutical cocrystal” or “cocrystal” of an active pharmaceutical ingredient (API), as used herein, refers to a crystalline material comprised of an API and one or more non-volative compound(s) (refered herein as a coformer). The API and the coformer interact through non-covalent forces in a crystal lattice.

As used herein and unless otherwise specified, the terms “solvate” and “solvated,” refer to a crystal form of a substance which contains solvent. The terms “hydrate” and “hydrated” refer to a solvate wherein the solvent comprises water. “Polymorphs of solvates” refers to the existence of more than one crystal form for a particular solvate composition. Similarly, “polymorphs of hydrates” refers to the existence of more than one crystal form for a particular hydrate composition. The term “desolvated solvate,” as used herein, refers to a crystal form of a substance which may be prepared by removing the solvent from a solvate.

As used herein and unless otherwise specified, the term “amorphous,” “amorphous form,” and related terms used herein, mean that the substance, component or product in question is not substantially crystalline as determined by X-ray diffraction. In particular, the term “amorphous form” describes a disordered solid form, i.e., a solid form lacking long range crystalline order. In certain embodiments, an amorphous form of a substance may be substantially free of other amorphous forms and/or crystal forms. In other embodiments, an amorphous form of a substance may contain less than about 1%, 2%, 3%, 4%, 5%, 10%, 15%, 20%, 25%, 30%, 35%, 40%, 45% or 50% of one or more other amorphous forms and/or crystal forms on a weight basis. In certain embodiments, an amorphous form of a substance may be physically and/or chemically pure. In certain embodiments, an amorphous form of a substance be about 99%, 98%, 97%, 96%, 95%, 94%, 93%, 92%, 91% or 90% physically and/or chemically pure.

Techniques for characterizing crystal forms and amorphous forms include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility measurements, dissolution measurements, elemental analysis, and Karl Fischer analysis. Characteristic unit cell parameters may be determined using one or more techniques such as, but not limited to, X-ray diffraction and neutron diffraction, including single-crystal diffraction and powder diffraction. Techniques useful for analyzing powder diffraction data include profile refinement, such as Rietveld refinement, which may be used, e.g., to analyze diffraction peaks associated with a single phase in a sample comprising more than one solid phase. Other methods useful for analyzing powder diffraction data include unit cell indexing, which allows one of skill in the art to determine unit cell parameters from a sample comprising crystalline powder.

As used herein and unless otherwise specified, the terms “about” and “approximately,” when used in connection with a numeric value or a range of values which is provided to characterize a particular solid form, e.g., a specific temperature or temperature range, such as, e.g., that describing a DSC or TGA thermal event, including, e.g., melting, dehydration, desolvation or glass transition events; a mass change, such as, e.g., a mass change as a function of temperature or humidity; a solvent or water content, in terms of, e.g., mass or a percentage; or a peak position, such as, e.g., in analysis by IR or Raman spectroscopy or XRPD; indicate that the value or range of values may deviate to an extent deemed reasonable to one of ordinary skill in the art while still describing the particular solid form. For example, in particular embodiments, the terms “about” and “approximately,” when used in this context and unless otherwise specified, indicate that the numeric value or range of values may vary within 25%, 20%, 15%, 10%, 9%, 8%, 7%, 6%, 5%, 4%, 3%, 2%, 1.5%, 1%, 0.5%, or 0.25% of the recited value or range of values.

As used herein and unless otherwise specified, a sample comprising a particular crystal form or amorphous form that is “substantially pure,” e.g., substantially free of other solid forms and/or of other chemical compounds, contains, in particular embodiments, less than about 25%, 20%, 15%, 10%, 9%, 8%, 7%, 6%, 5%, 4%, 3%, 2%, 1%, 0.75%, 0.5%, 0.25% or 0.1% percent by weight of one or more other solid forms and/or of other chemical compounds.

As used herein and unless otherwise specified, a sample or composition that is “substantially free” of one or more other solid forms and/or other chemical compounds means that the composition contains, in particular embodiments, less than about 25%, 20%, 15%, 10%, 9%, 8%, 7%, 6%, 5%, 4%, 3%, 2%, 1%, 0.75%, 0.5%, 0.25% or 0.1% percent by weight of one or more other solid forms and/or other chemical compounds.

As used herein, and unless otherwise specified, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a patient with such a disease or disorder. In some embodiments, the terms refer to the administration of a compound provided herein, with or without other additional active agent, after the onset of symptoms of the particular disease.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound provided herein, with or without other additional active compound, prior to the onset of symptoms, particularly to patients at risk of diseases or disorders provided herein. The terms encompass the inhibition or reduction of a symptom of the particular disease. Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.”

As used herein, and unless otherwise specified, the terms “manage,” “managing” and “management” refer to preventing or slowing the progression, spread or worsening of a disease or disorder, or of one or more symptoms thereof. Often, the beneficial effects that a patient derives from a prophylactic and/or therapeutic agent do not result in a cure of the disease or disorder. In this regard, the term “managing” encompasses treating a patient who had suffered from the particular disease in an attempt to prevent or minimize the recurrence of the disease.

As used herein, and unless otherwise specified, a “therapeutically effective amount” of a compound is an amount sufficient to provide a therapeutic benefit in the treatment or management of a disease or disorder, or to delay or minimize one or more symptoms associated with the disease or disorder. A therapeutically effective amount of a compound means an amount of therapeutic agent, alone or in combination with other therapies, which provides a therapeutic benefit in the treatment or management of the disease or disorder. The term “therapeutically effective amount” can encompass an amount that improves overall therapy, reduces or avoids symptoms or causes of disease or disorder, or enhances the therapeutic efficacy of another therapeutic agent.

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

The term “composition” as used herein is intended to encompass a product comprising the specified ingredients (and in the specified amounts, if indicated), as well as any product which results, directly or indirectly, from combination of the specified ingredients in the specified amounts. By “pharmaceutically acceptable” it is meant that the diluent, excipient or carrier must be compatible with the other ingredients of the formulation and not deleterious to the recipient thereof.

### 5. DETAILED DESCRIPTION

This disclosure relates to solid forms comprising salts and cocrystals of Compound 1, which is 3-(5-amino-2-methyl-4-oxo-4H-quinazolin-3-yl)-piperidine-2,6-dione, and stereoisomers thereof, as well as methods of using, and compositions comprising, a solid form comprising salts and cocrystals of Compound 1 or a stereoisomer thereof. For example, the present disclosure encompasses the in vitro and in vivo use of a solid form comprising salts and cocrystals of Compound 1, and the incorporation of a solid form comprising salts and cocrystals of Compound 1 into pharmaceutical compositions and single unit dosage forms useful in the treatment and prevention of a variety of diseases and disorders.

### 5.1. SOLID FORMS OF COMPOUND 1

In one embodiment, provided herein are solid forms of salts of Compound 1 or cocrystals comprising Compound 1.

Compound 1 is readily prepared using the methods as described U.S. Pat. No. 7,635,700, the disclosure of which is incorporated herein by reference in its entirety.

In certain embodiments, the solid forms provided herein include solid forms comprising salts of Compound 1, such as a besylate salt (benzenesulfonate salt), an esylate salt (ethanesulfonate salt), a mesylate salt (methanesulfonate salt), a napadisylate salt (naphthalene-1,5-disulfonic acid salt), a napsilate salt (naphthalene-2-sulfonic acid salt), a thiocyanate salt (thiocyanic acid salt), a tosylate salt (p-toluenesulfonate salt) and a sulfate salt (sulfuric acid salt).

In one embodiment, provided herein is Form A of a besylate salt of Compound 1.

In one embodiment, provided herein are solid forms of an esylate salt of Compound 1. In one embodiment, provided herein is polymorph Form A of an esylate salt of Compound 1. In one embodiment, provided herein is polymorph Form B of an esylate salt of Compound 1.

In one embodiment, provided herein are solid forms of a mesylate salt of Compound 1. In one embodiment, provided herein is polymorph Form A of a mesylate salt of Compound 1. In one embodiment, provided herein is polymorph Form B of a mesylate salt of Compound 1.

In one embodiment, provided herein is Form A of a napadisylate salt of Compound 1.

In one embodiment, provided herein is Form A of a napsilate salt of Compound 1.

In one embodiment, provided herein is Form A of a sulfate salt of Compound 1.

In one embodiment, provided herein are solid forms of a thiocyanate salt of Compound 1. In one embodiment, provided herein is polymorph Form A of a thiocyanate salt of Compound 1. In one embodiment, provided herein is polymorph Form B of a thiocyanate salt of Compound 1.

In one embodiment, provided herein are solid forms of a tosylate salt of Compound 1. In one embodiment, provided herein is polymorph Form A of a tosylate salt of Compound 1. In one embodiment, provided herein is polymorph Form B of a tosylate salt of Compound 1.

In certain embodiments, solid forms provided herein include cocrystals comprising Compound 1, such as solids forms of Compound 1 acetic acid cocrystal, Compound 1 isethionic acid cocrystal, Compound 1 lauric acid cocrystal, Compound 1 maleic acid cocrystal and Compound 1 phosphoric acid cocrystal.

In one embodiment, provided herein is Compound 1 acetic acid cocrystal Form A.

In one embodiment, provided herein are solid forms of Compound 1 isethionic acid cocrystal. In one embodiment, provided herein is Compound 1 isethionic acid cocrystal Form A. In one embodiment, provided herein is Compound 1 isethionic acid cocrystal Form B.

In one embodiment, provided herein is Compound 1 lauric acid cocrystal Form A.

In one embodiment, provided herein is Compound 1 maleic acid cocrystal Form A.

In one embodiment, provided herein is Compound 1 phosphoric acid cocrystal Form A.

Solid forms comprising Compound 1 can be prepared by the methods described herein, including the methods described in the Examples below, or by techniques known in the art, including heating, cooling, freeze drying, lyophilization, quench cooling the melt, rapid solvent evaporation, slow solvent evaporation, solvent recrystallization, antisolvent addition, slurry recrystallization, crystallization from the melt, desolvation, recrystallization in confined spaces such as, e.g., in nanopores or capillaries, recrystallization on surfaces or templates such as, e.g., on polymers, recrystallization in the presence of additives, such as, e.g., cocrystal counter-molecules, desolvation, dehydration, rapid cooling, slow cooling, exposure to solvent and/or water, drying, including, e.g., vacuum drying, vapor diffusion, sublimation, grinding (including, e.g., cryo-grinding, solvent-drop grinding or liquid assisted grinding), microwave-induced precipitation, sonication-induced precipitation, laser-induced precipitation and precipitation from a supercritical fluid. The particle size of the resulting solid forms, which can vary, (e.g., from nanometer dimensions to millimeter dimensions), can be controlled, e.g., by varying crystallization conditions, such as, e.g., the rate of crystallization and/or the crystallization solvent system, or by particle-size reduction techniques, e.g., grinding, milling, micronizing or sonication.

While not intending to be bound by any particular theory, certain solid forms are characterized by physical properties, e.g., stability, solubility and dissolution rate, appropriate for pharmaceutical and therapeutic dosage forms. Moreover, while not wishing to be bound by any particular theory, certain solid forms are characterized by physical properties (e.g., density, compressibility, hardness, morphology, cleavage, stickiness, solubility, water uptake, electrical properties, thermal behavior, solid-state reactivity, physical stability, and chemical stability) affecting particular processes (e.g., yield, filtration, washing, drying, milling, mixing, tableting, flowability, dissolution, formulation, and lyophilization) which make certain solid forms suitable for the manufacture of a solid dosage form. Such properties can be determined using particular analytical chemical techniques, including solid-state analytical techniques (e.g., X-ray diffraction, microscopy, spectroscopy and thermal analysis), as described herein and known in the art.

Certain embodiments herein provide compositions comprising one or more of the solid forms. Certain embodiments provide compositions of one or more solid forms in combination with other active ingredients. Certain embodiments provide methods of using these compositions in the treatment, prevention or management of diseases and disorders including, but not limited to, the diseases and disorders provided herein.

Solid forms provided herein may also comprise unnatural proportions of atomic isotopes at one or more of the atoms in Compound 1. For example, the compound may be radiolabeled with radioactive isotopes, such as for example deuterium, tritium (³H), iodine-125 (¹²⁵I) sulfur-35 (³⁵S), or carbon-14 (¹⁴C). Radiolabeled compounds are useful as therapeutic agents, e.g., cancer therapeutic agents, research reagents, e.g., binding assay reagents, and diagnostic agents, e.g., in vivo imaging agents. All isotopic variations of Compound 1, whether radioactive or not, are intended to be encompassed within the scope of the embodiments provided herein.

### 5.1.1. Besylate Salt of Compound 1

Certain embodiments herein provide Form A of besylate salt of Compound 1. In one embodiment, Form A of besylate salt of Compound 1 provided herein is prepared by stoichiometric precipitation of the benzenesulfonic acid and Compound 1 in acetone at room temperature.

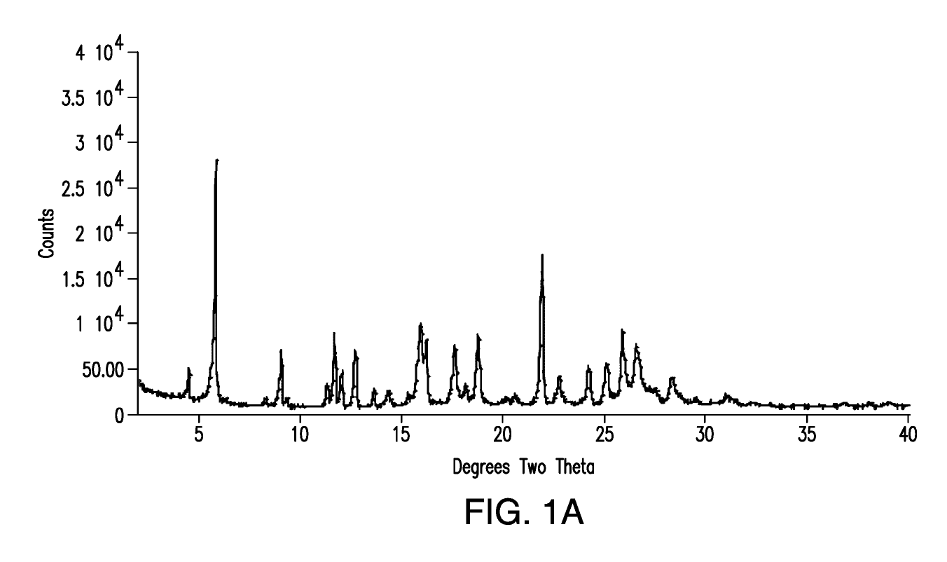

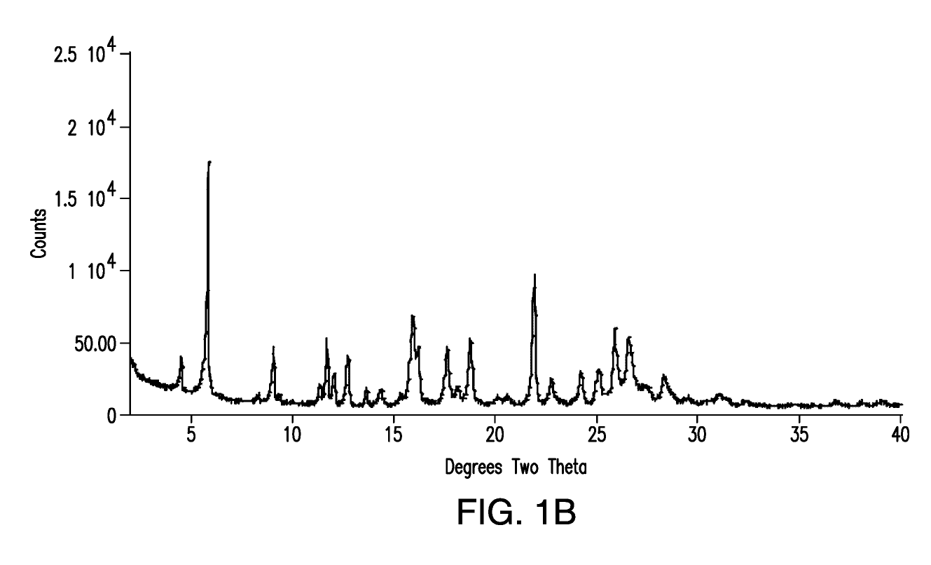

A representative XRPD pattern of Form A of the besylate salt of Compound 1 is shown in FIG. 1A.

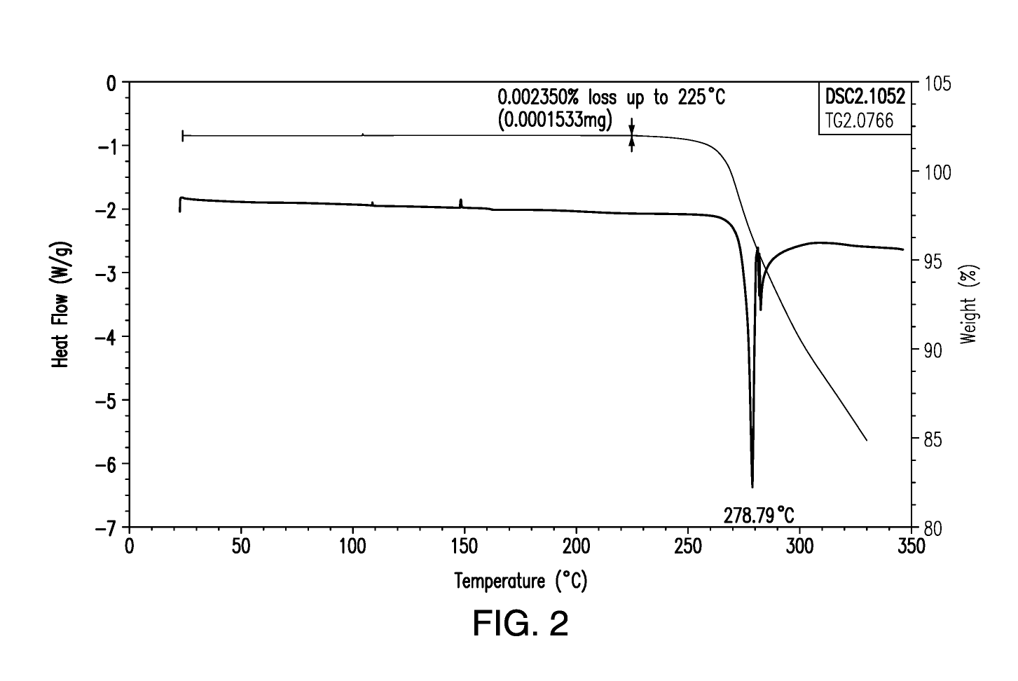

Representative thermal characteristics for Form A of the besylate salt of Compound 1 are shown in FIG. 2. A representative TGA thermogram comprises a weight loss of about 0.002% based on the total weight of sample upon heating up to about 225° C. In certain embodiments, the aforementioned weight loss comprises a loss of solvent, such as, e.g., a loss of acetone.

In certain embodiments, Form A of the besylate salt of Compound 1 is characterized by a DSC thermogram comprising a thermal event at approximate temperature 279° C. In certain embodiments, Form A of the besylate salt of Compound 1 is characterized by a DSC thermogram comprising a thermal event at approximate temperature 278.79° C. A representative DSC thermogram is shown in FIG. 2.

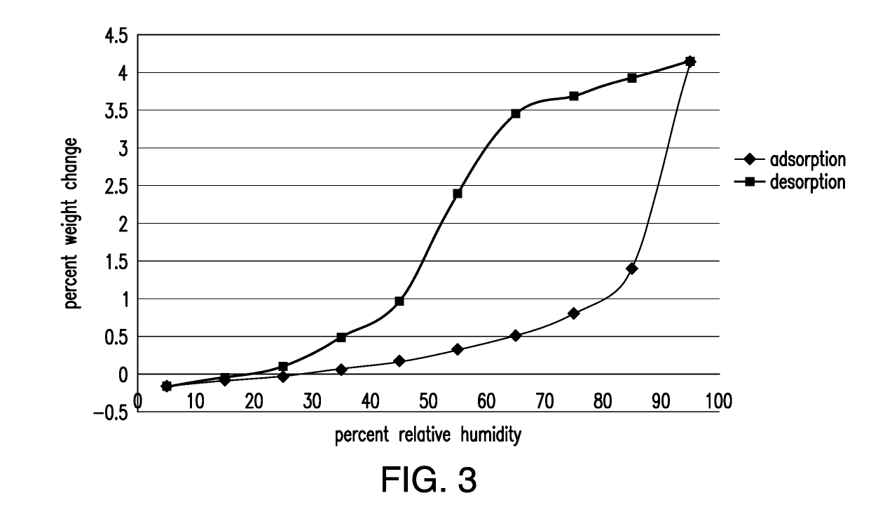

A representative dynamic vapor sorption (DVS) isotherm plot for Form A of the besylate salt of Compound 1 is shown in FIG. 3. In certain embodiments, when the relative humidity (“RH”) is increased from about 5% to about 95% RH, Form A of the besylate salt of Compound 1 exhibits about 4.31% weight gain. In certain embodiments, when the relative humidity (“RH”) is decreased from about 95% to about 5% RH, Form A of the besylate salt of Compound 1 exhibits about 4.31% weight loss.

FIG. 1B provides a post-DVS X-ray Powder Diffraction (“XRPD”) pattern of Form A of besylate salt of 3-(5-amino-2-methyl-4-oxo-4H-quinazolin-3-yl)-piperidine-2,6-dione

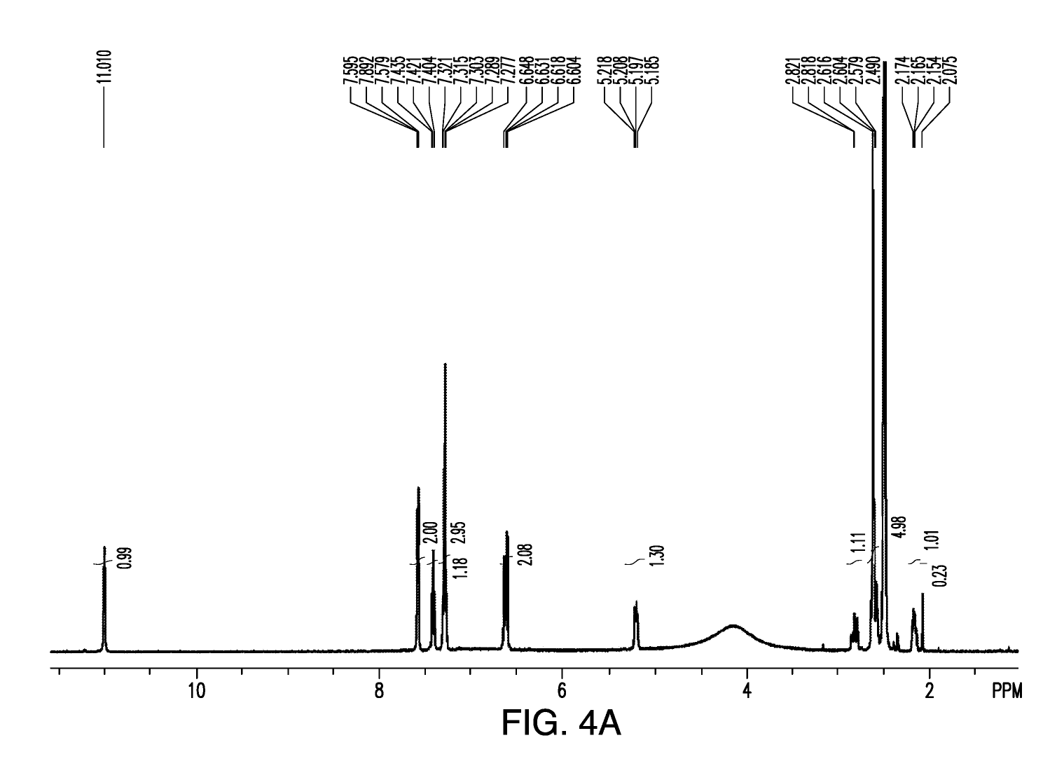

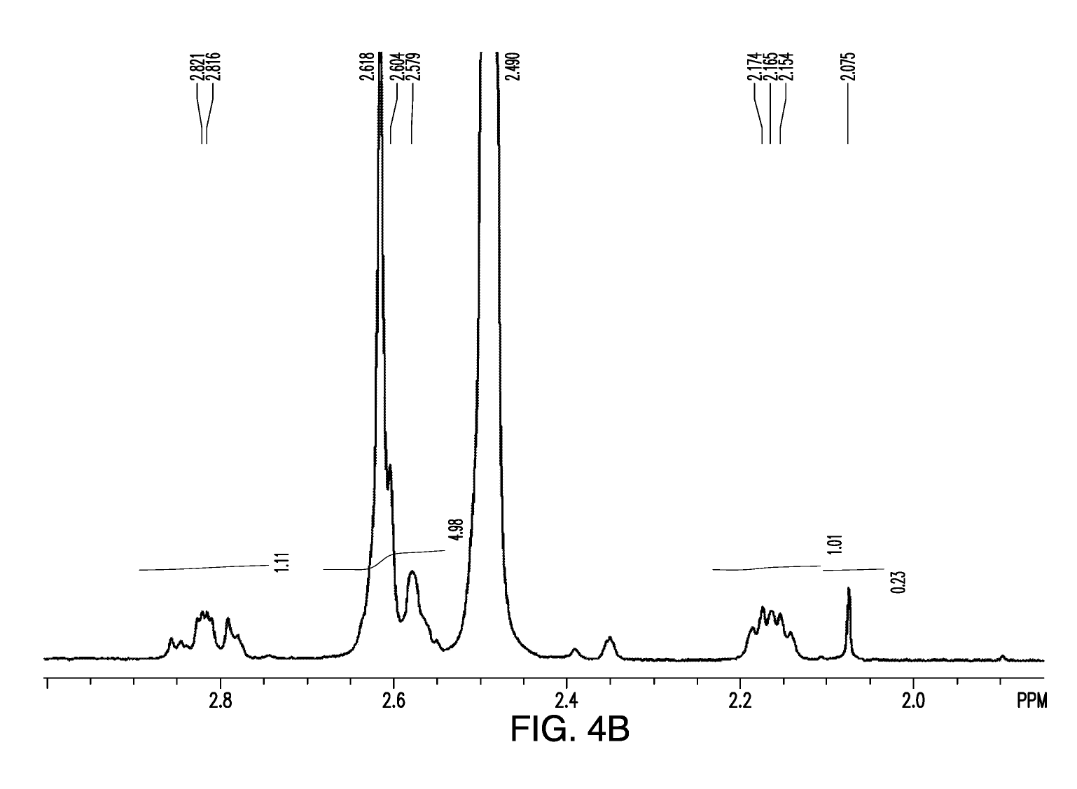

In certain embodiments, the chemical profile of a sample of Form A of the besylate salt of Compound 1 is characterized by solution NMR analysis. A representative ¹H NMR spectrum of a sample of Form A of the besylate salt of Compound 1 dissolved in DMSO-d₆ is provided as FIGS. 4A, 4B and 4C.

### 5.1.2. Esylate Salt of Compound 1

Certain embodiments herein provide Form A and Form B of esylate salt of Compound 1.

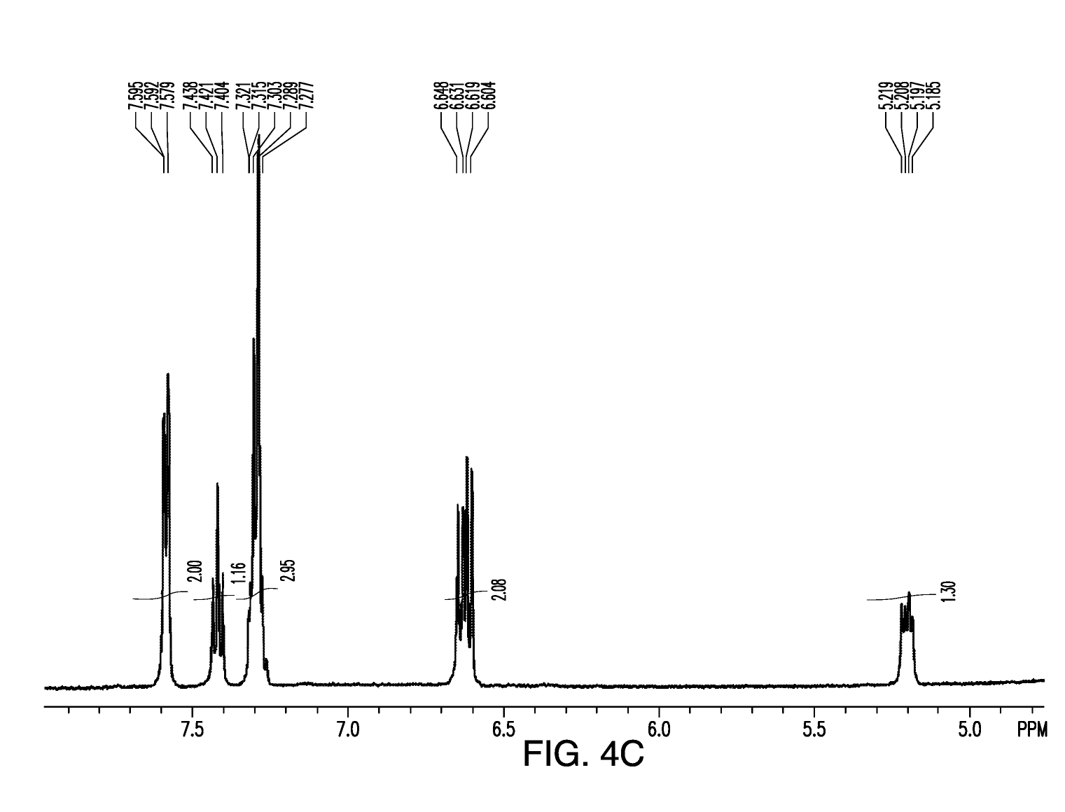

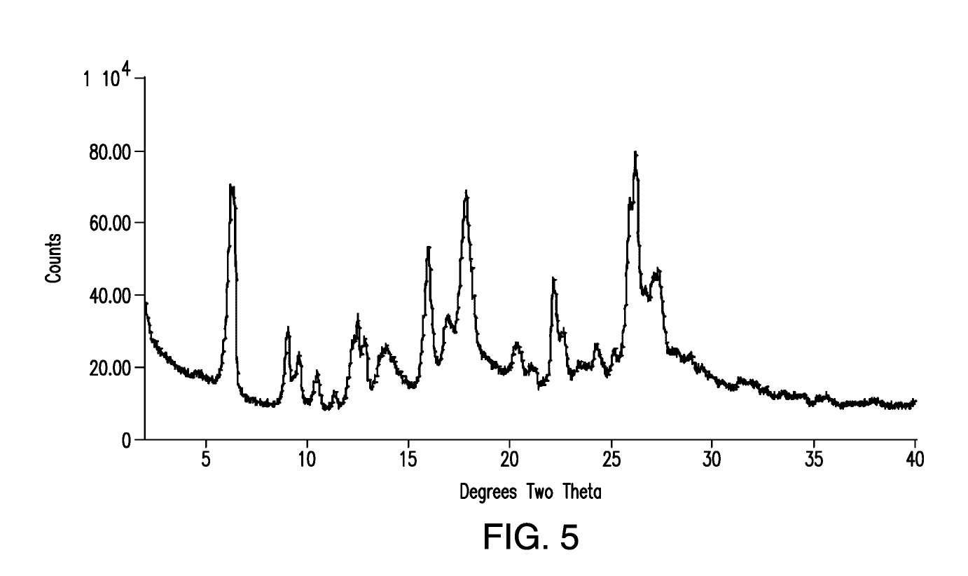

A representative XRPD pattern of Form B of the esylate salt of Compound 1 is shown in FIG. 5.

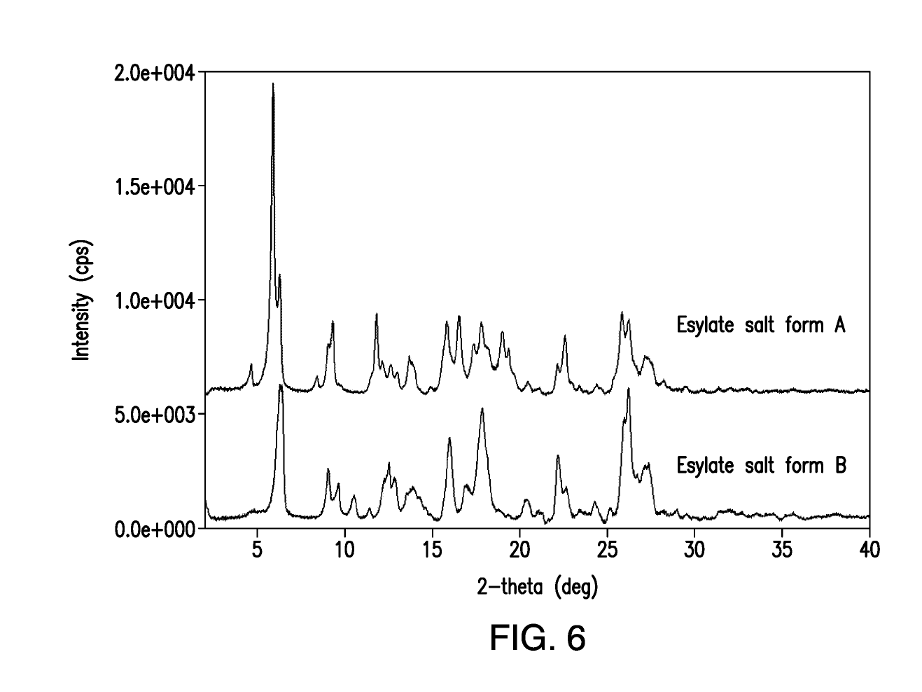

Representative XRPD patterns of esylate salt Form A and Form B of Compound 1 are overlaid and shown in FIG. 6.

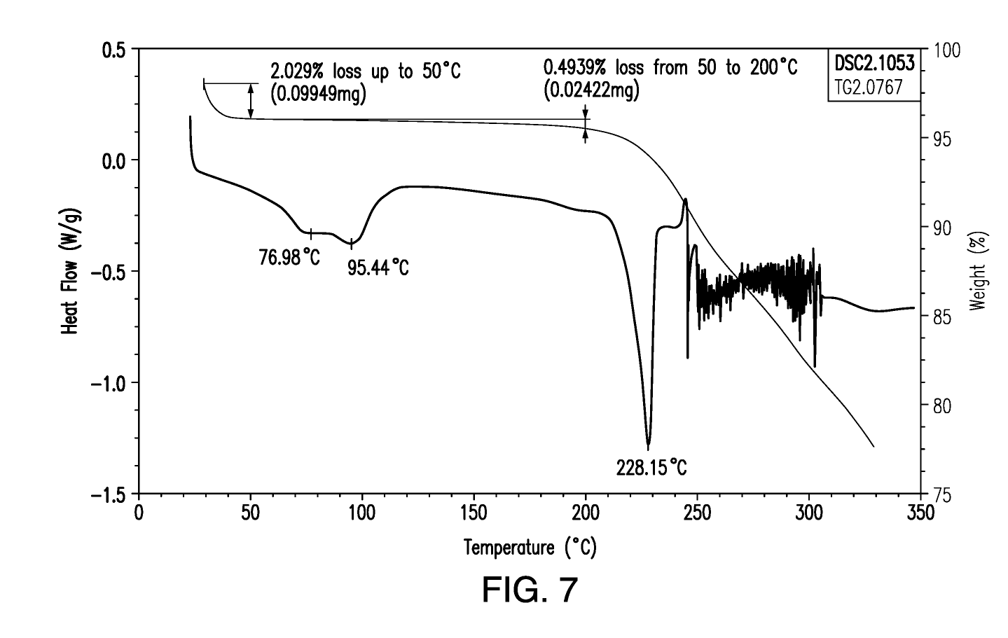

Representative thermal characteristics for Form B of the esylate salt of Compound 1 are shown in FIG. 7. A representative TGA thermogram comprises a weight loss of about 2.03% based on the total weight of sample upon heating up to about 50° C., and about 0.49% upon heating from about 50° C. to 200° C. In certain embodiments, the aforementioned weight loss comprises a loss of solvent, such as, e.g., a loss of acetone.

In certain embodiments, Form B of the esylate salt of Compound 1 is characterized by a DSC thermogram comprising one or more thermal events with the following approximate temperatures: 77, 95, and 228° C. In certain embodiments, Form B of the esylate salt of Compound 1 is characterized by a DSC thermogram comprising one or more thermal events with the following approximate temperatures: 76.98, 95.44, and 228.15° C. A representative DSC thermogram is shown in FIG. 7.

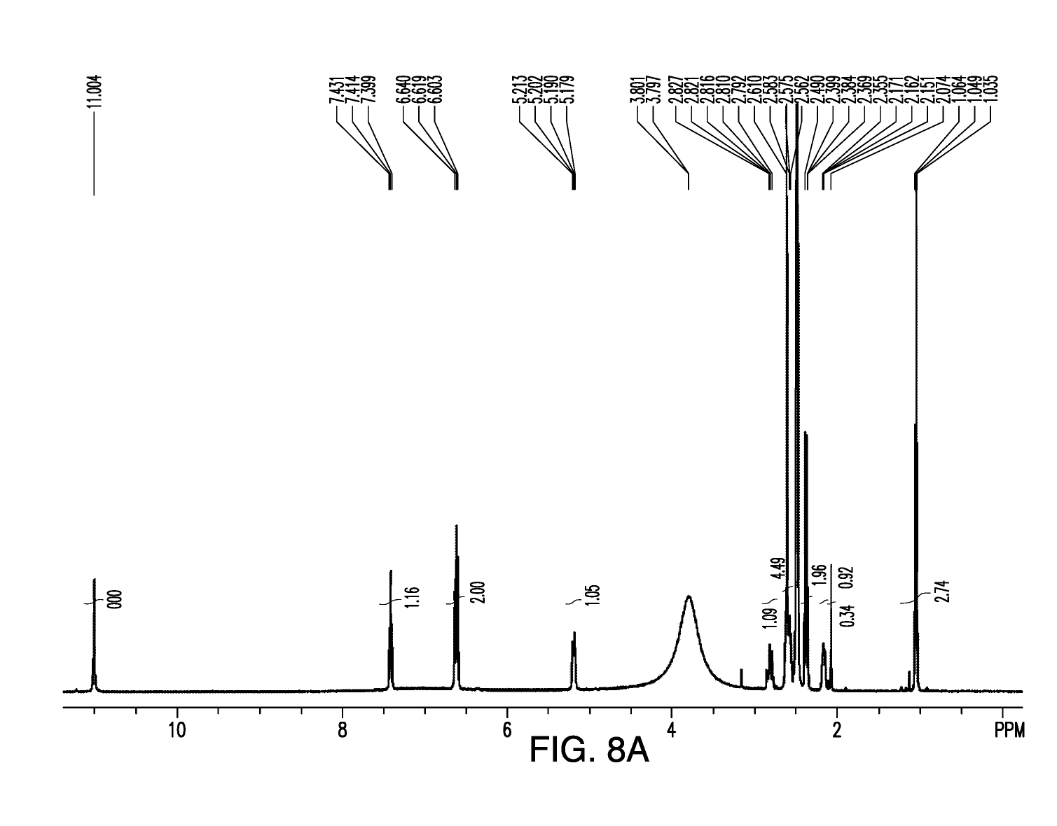

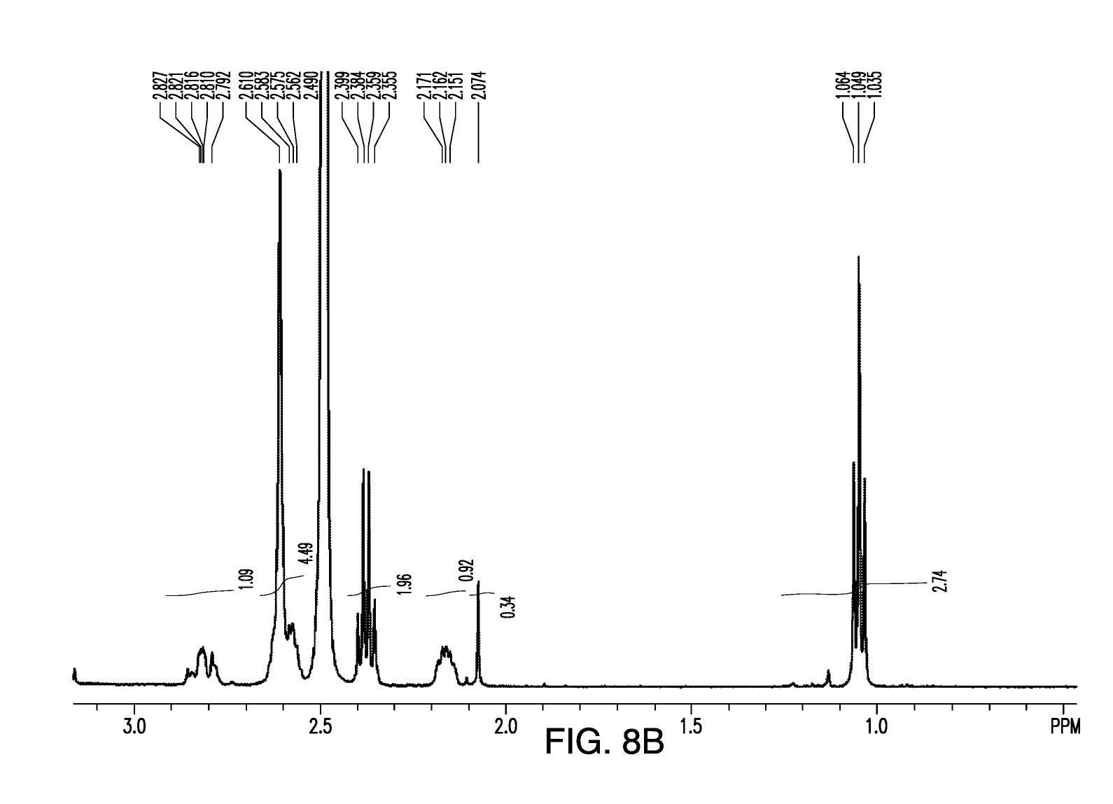

In certain embodiments, the chemical profile of a sample of Form B of the esylate salt of Compound 1 is characterized by solution NMR analysis. A representative ¹H NMR spectrum of a sample of Form B of the esylate salt of Compound 1 dissolved in DMSO-d₆ is provided as FIGS. 8A and 8B.

### 5.1.3. Napadisylate Salt of Compound 1

Certain embodiments herein provide Form A of napadisylate salt of Compound 1.

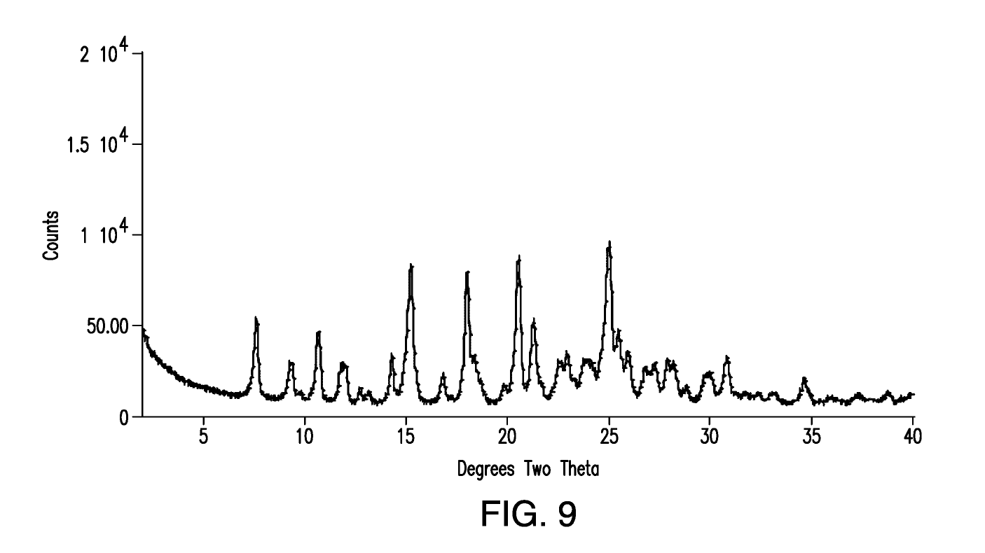

A representative XRPD pattern of Form A of the napadisylate salt of Compound 1 is shown in FIG. 9.

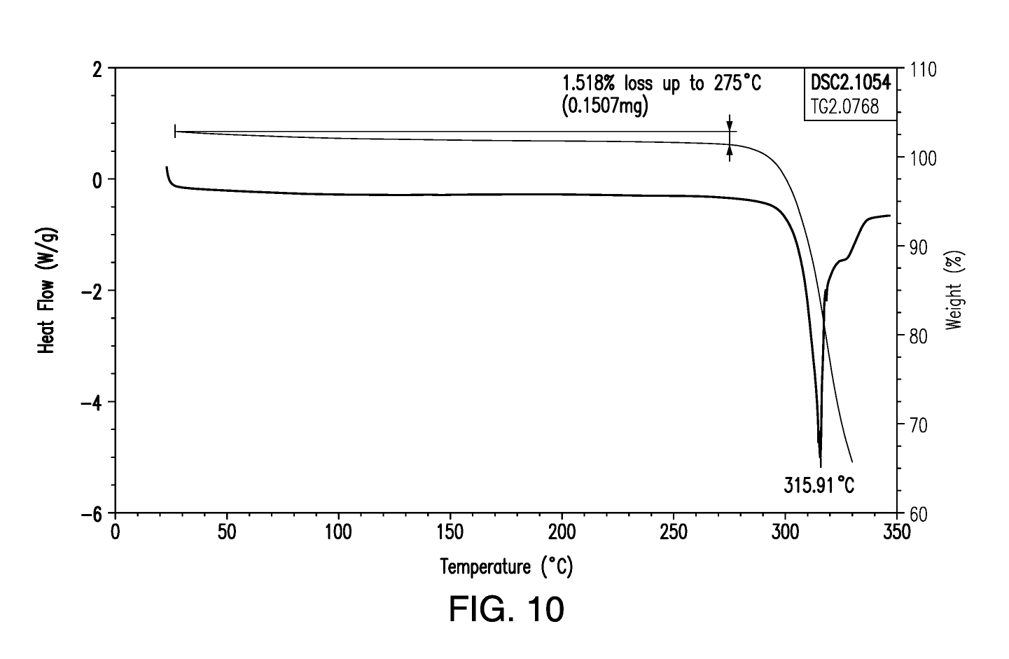

Representative thermal characteristics for Form A of the napadisylate salt of Compound 1 are shown in FIG. 10. A representative TGA thermogram comprises a weight loss of about 1.52% based on the total weight of sample upon heating up to about 275° C. In certain embodiments, the aforementioned weight loss comprises a loss of solvent, such as, e.g., a loss of acetone.

In certain embodiments, Form A of the napadisylate salt of Compound 1 is characterized by a DSC thermogram comprising an endothermic event at an approximate temperature of 316° C. In certain embodiments, Form A of the napadisylate salt of Compound 1 is characterized by a DSC thermogram comprising an endothermic event at an approximate temperature of 315.91° C. A representative DSC thermogram is shown in FIG. 10.

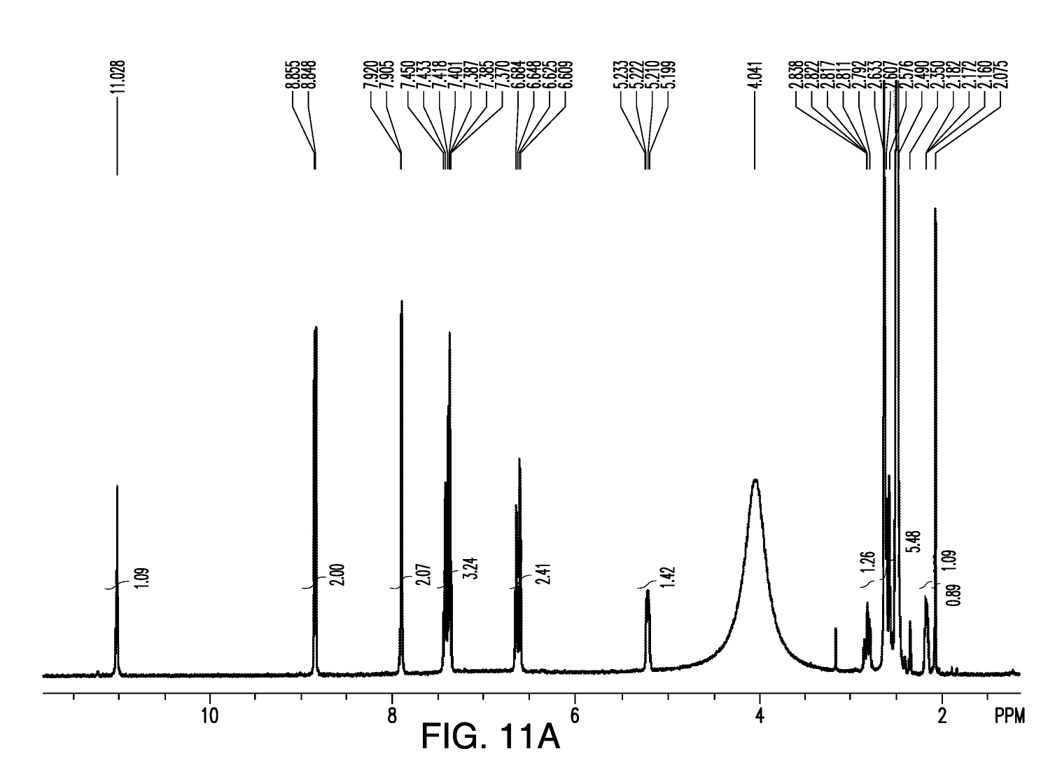

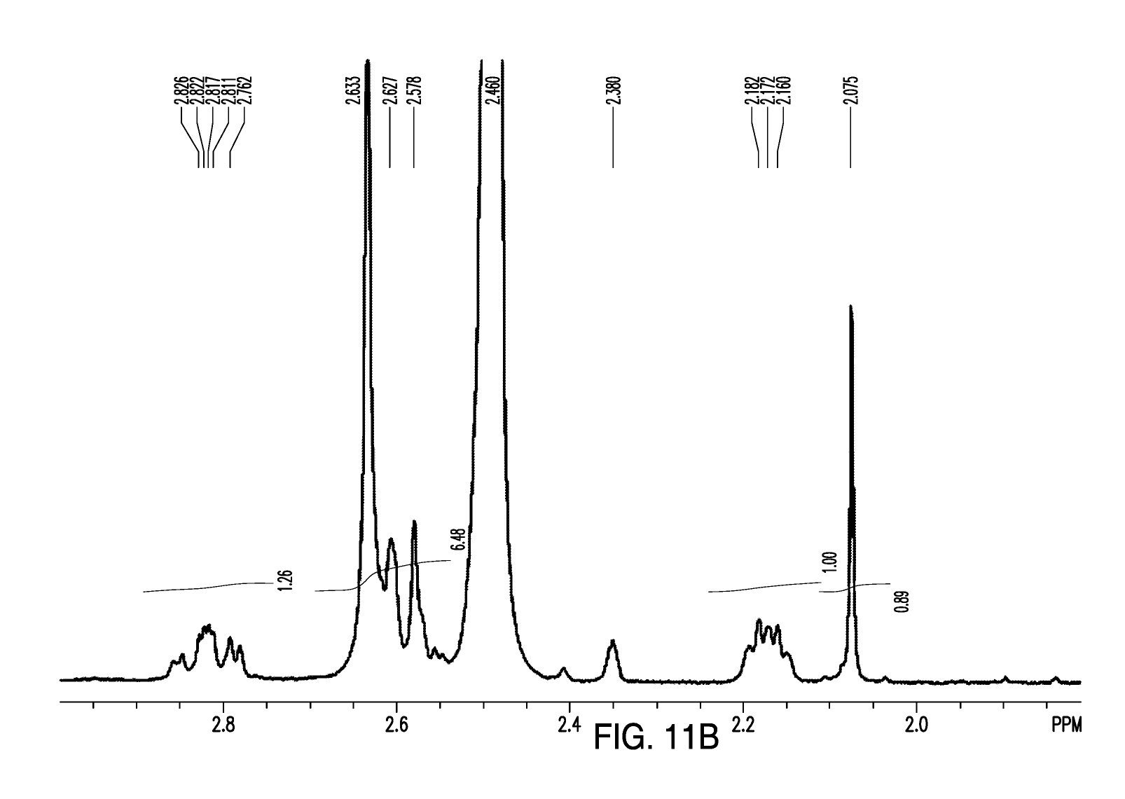

In certain embodiments, the chemical profile of a sample of Form A of the napadisylate salt of Compound 1 is characterized by solution NMR analysis. A representative ¹H NMR spectrum of a sample of Form A of the napadisylate salt of Compound 1 dissolved in DMSO-d₆ is provided as FIGS. 11A and 11B.

### 5.1.4. Napsilate Salt of Compound 1

Certain embodiments herein provide Form A of napsilate salt of Compound 1.

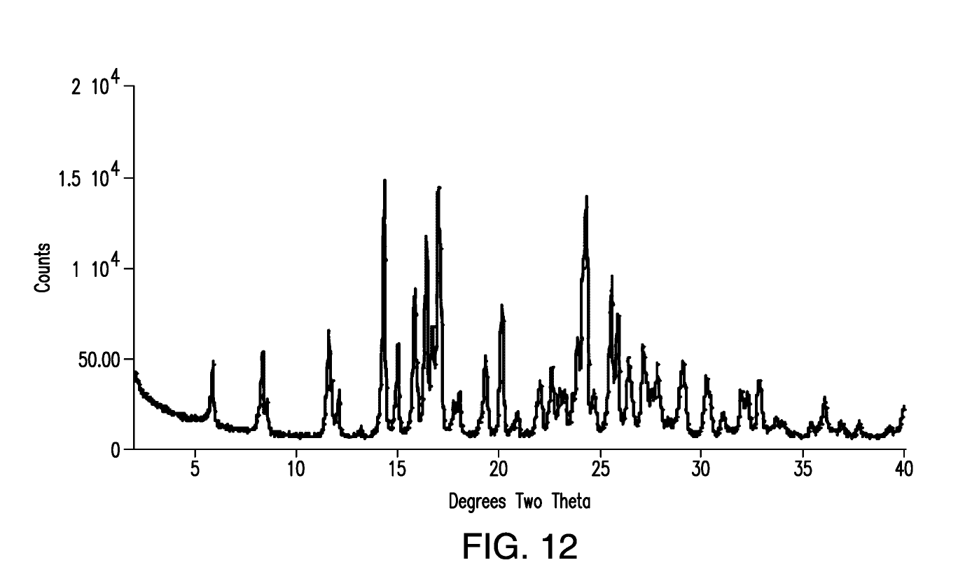

A representative XRPD pattern of Form A of the napsilate salt of Compound 1 is shown in FIG. 12.

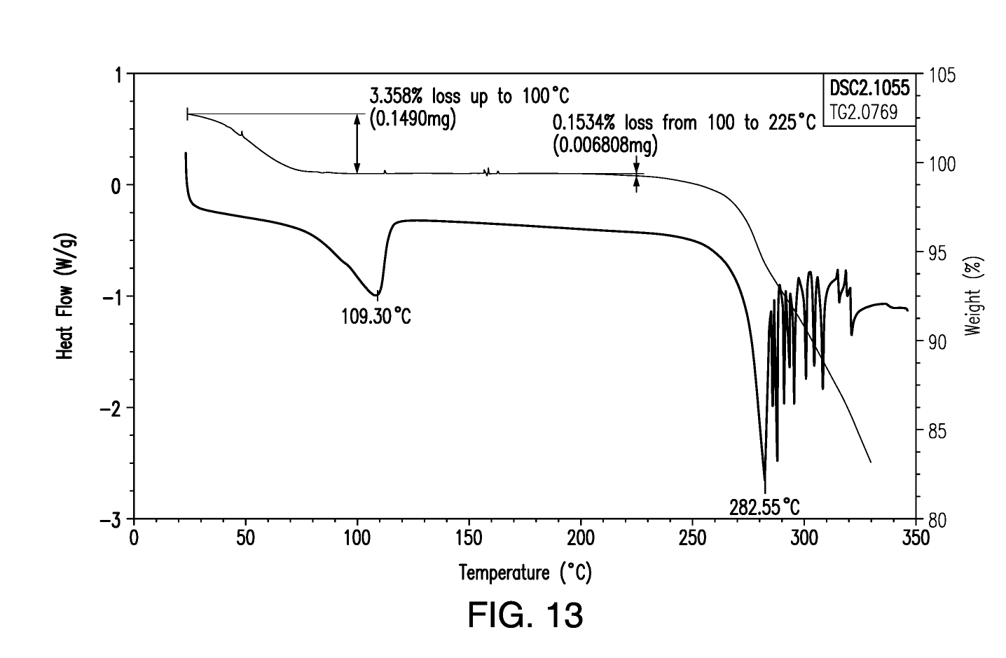

Representative thermal characteristics for Form A of the napsilate salt of Compound 1 are shown in FIG. 13. A representative TGA thermogram comprises a weight loss of about 3.36% based on the total weight of sample upon heating up to about 100° C., and about 0.15% weight loss from 100 to 225° C. In certain embodiments, the aforementioned weight loss comprises a loss of solvent, such as, e.g., a loss of acetone.

In certain embodiments, Form A of the napsilate salt of Compound 1 is characterized by a DSC thermogram comprising one or more endothermic events at approximate temperatures of 109 and 283° C. In certain embodiments, Form A of the napsilate salt of Compound 1 is characterized by a DSC thermogram comprising one or more endothermic events at approximate temperatures of 109.30 and 282.55° C. A representative DSC thermogram is shown in FIG. 13.

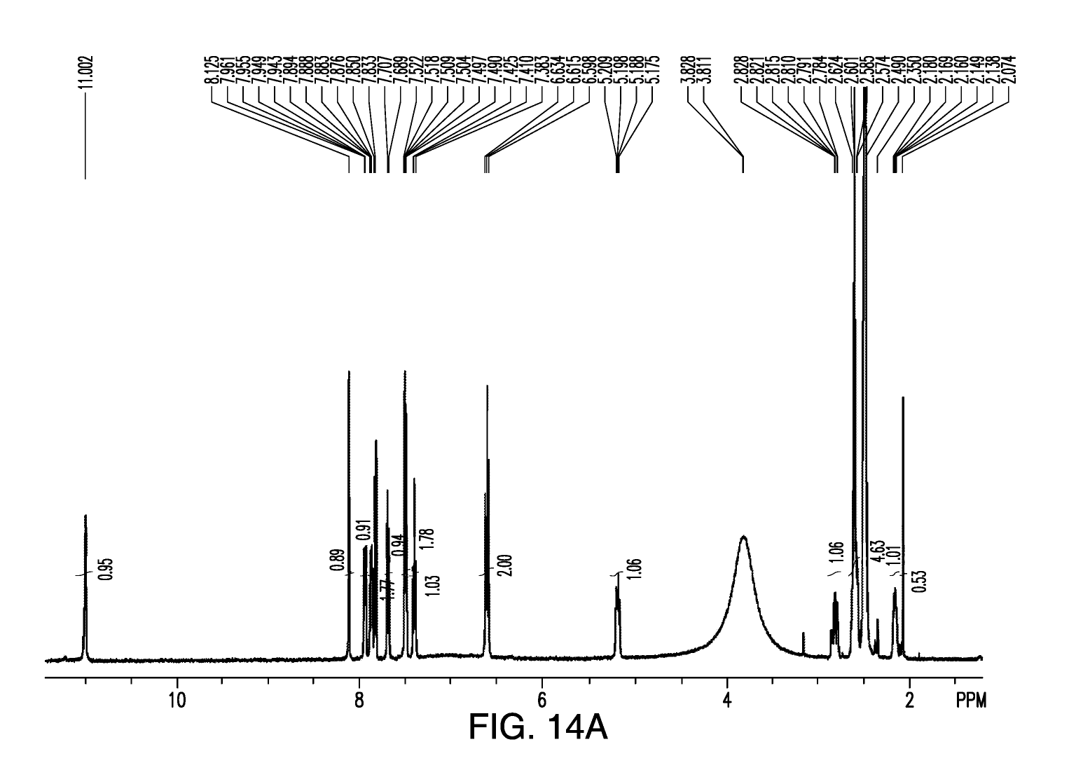

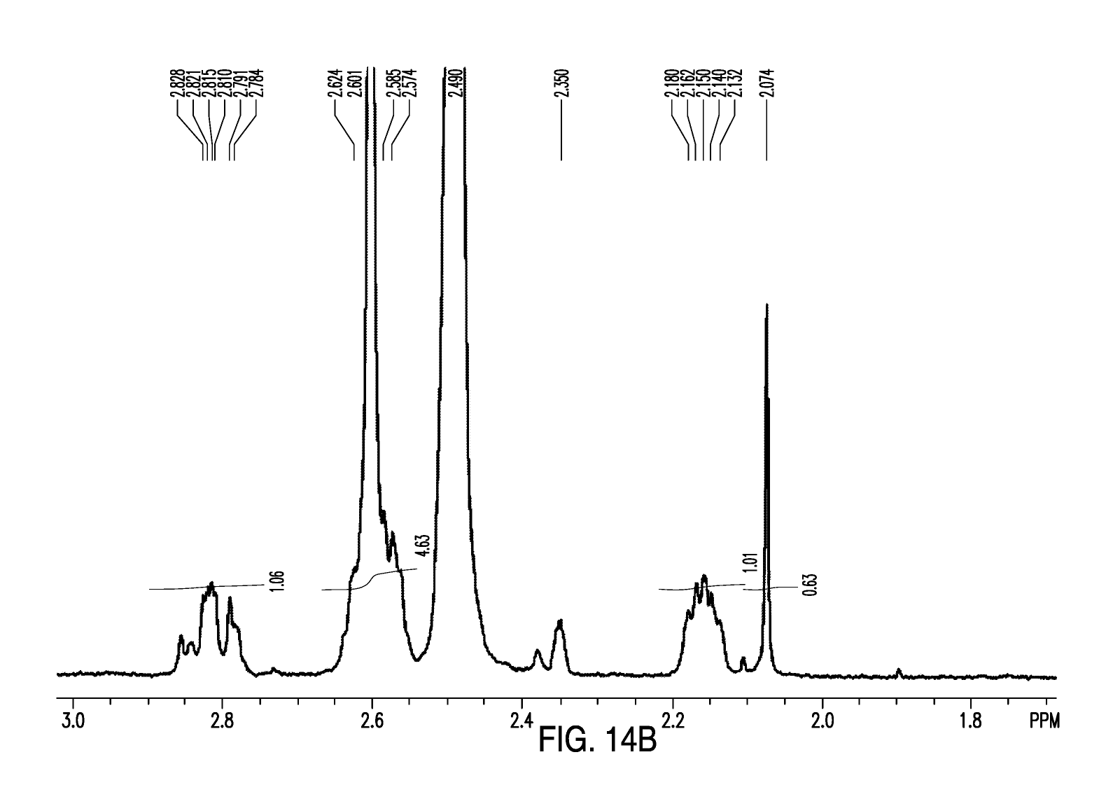

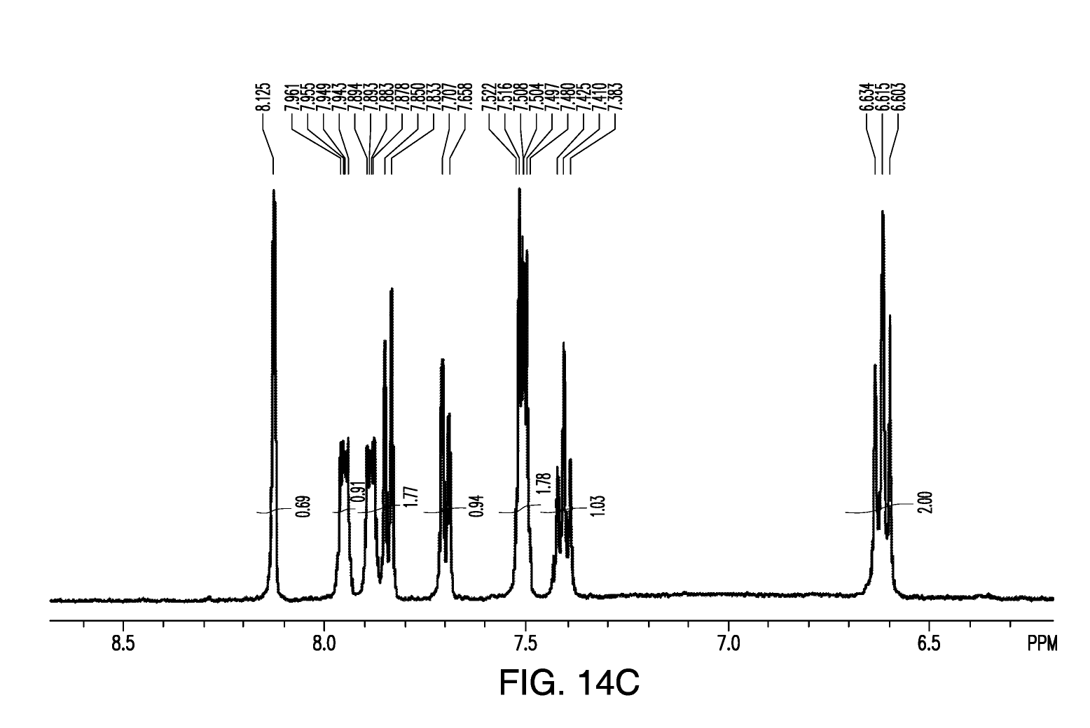

In certain embodiments, the chemical profile of a sample of Form A of the napsilate salt of Compound 1 is characterized by solution NMR analysis. A representative ¹H NMR spectrum of a sample of Form A of the napsilate salt of Compound 1 dissolved in DMSO-d₆ is provided as FIGS. 14A 14B and 14C.

### 5.1.5. Mesylate Salt of Compound 1

Certain embodiments herein provide Form A and Form B of mesylate salt of Compound 1.

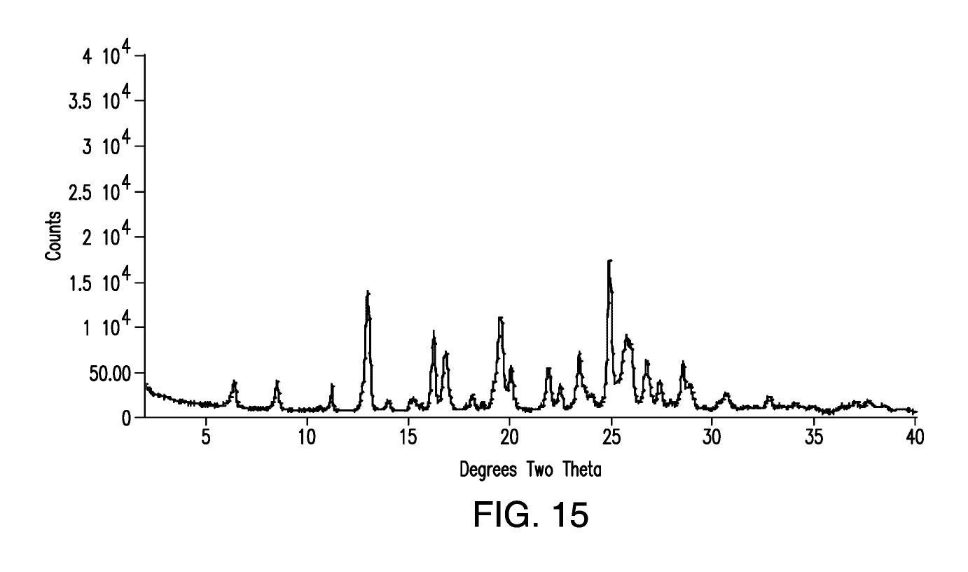

A representative XRPD pattern of Form A of the mesylate salt of Compound 1 is shown in FIG. 15.

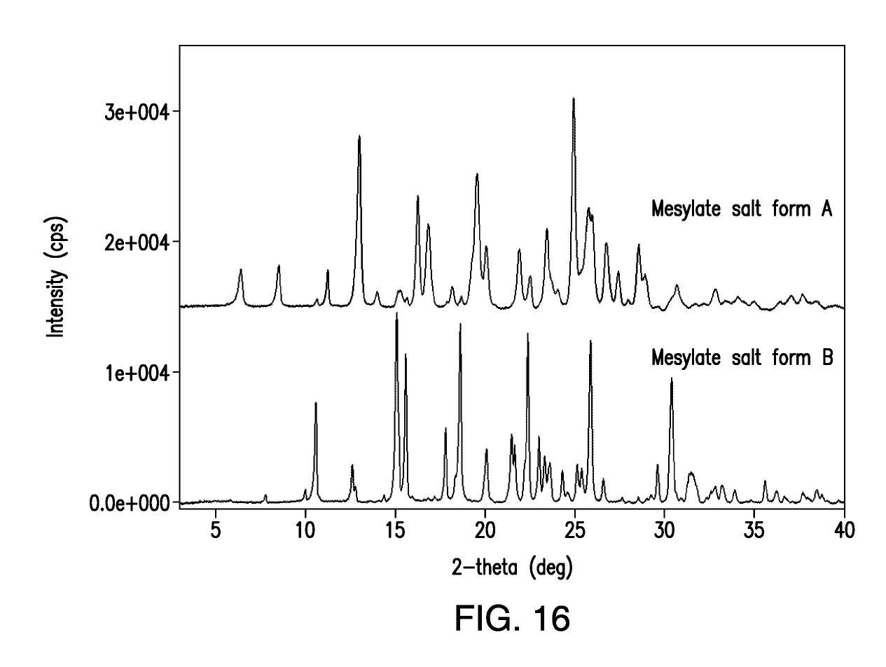

Representative XRPD patterns of Compound 1 mesylate salt Form A and Form B are overlaid and shown in FIG. 16.

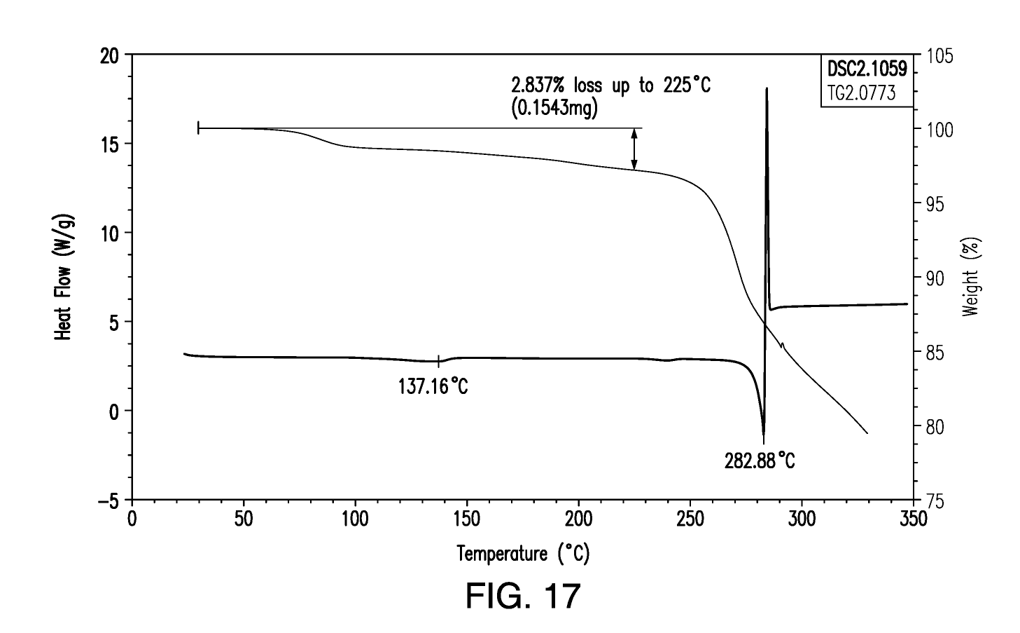

Representative thermal characteristics for Form A of the mesylate salt of Compound 1 are shown in FIG. 17. A representative TGA thermogram comprises a weight loss of about 2.84% based on the total weight of sample upon heating up to about 225° C. In certain embodiments, the aforementioned weight loss comprises a loss of solvent, such as, e.g., a loss of acetone.

In certain embodiments, Form A of the mesylate salt of Compound 1 is characterized by a DSC thermogram comprising one or more thermal events with the following approximate temperatures: 137 and 283° C. In certain embodiments, Form A of the mesylate salt of Compound 1 is characterized by a DSC thermogram comprising one or more thermal events with the following approximate temperatures: 137.16 and 282.88° C. A representative DSC thermogram is shown in FIG. 17.

In certain embodiments, the chemical profile of a sample of Form A of the mesylate salt of Compound 1 is characterized by solution NMR analysis. A representative ¹H NMR spectrum of a sample of Form A of the mesylate salt of Compound 1 dissolved in DMSO-d₆ is provided as FIGS. 18A and 18B.

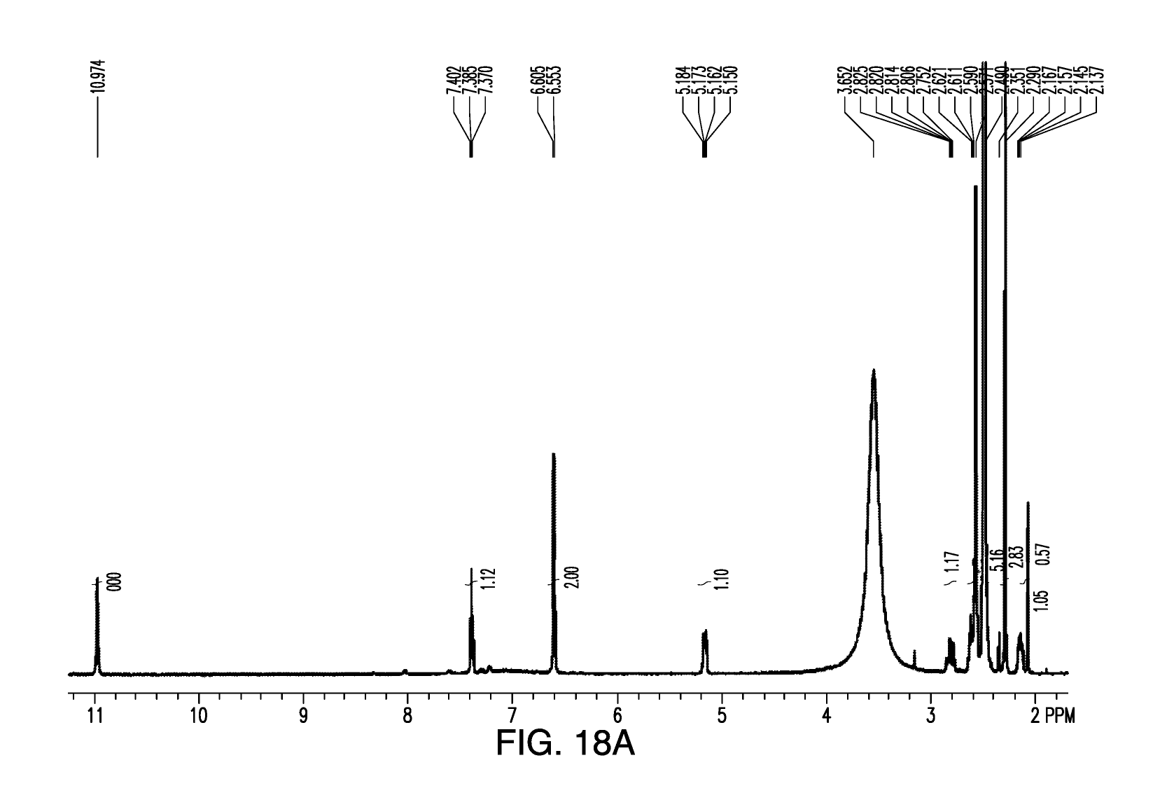

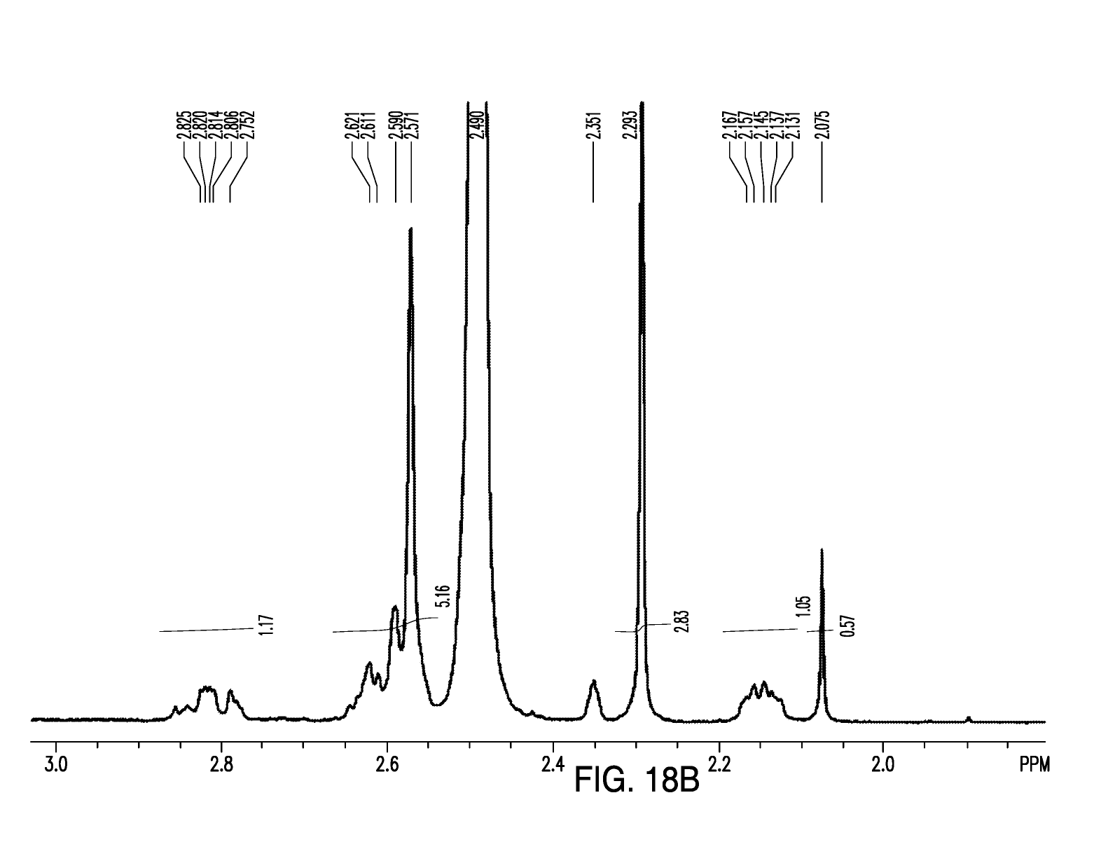

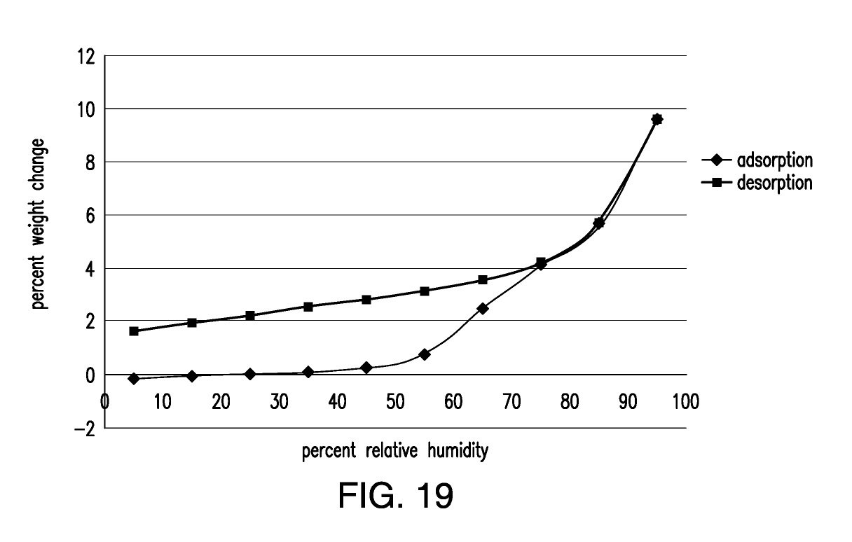

A representative dynamic vapor sorption (DVS) isotherm plot for Form A of the mesylate salt of Compound 1 is shown in FIG. 19. In certain embodiments, when the relative humidity (“RH”) is increased from about 5% to about 95% RH, Form A of the mesylate salt of Compound 1 exhibits about 9.79% weight gain. In certain embodiments, when the relative humidity (“RH”) is decreased from about 95% to about 5% RH, Form A of the mesylate salt of Compound 1 exhibits about 7.98% weight loss.

### 5.1.6. Sulfate Salt of Compound 1

Certain embodiments herein provide Form A of sulfate salt of Compound 1.

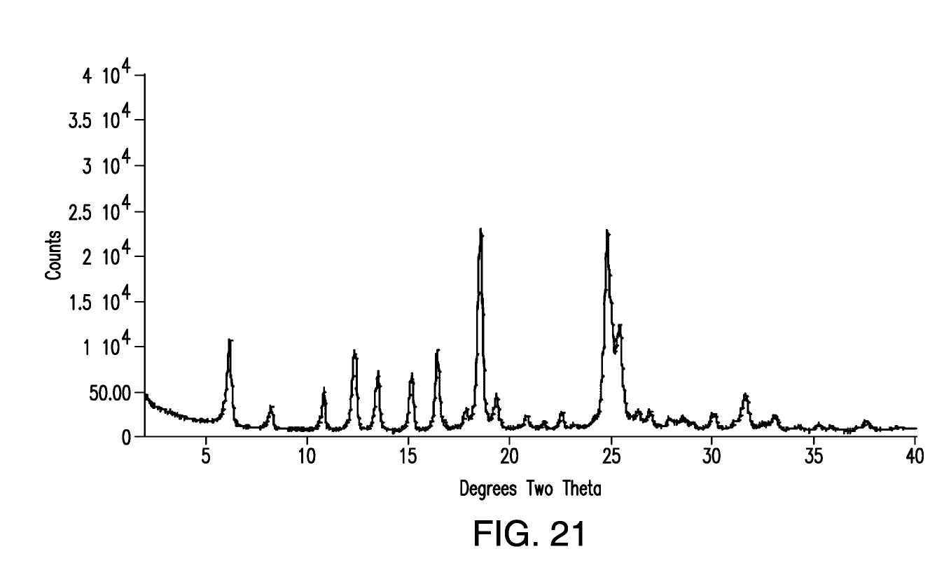

A representative XRPD pattern of Form A of the sulfate salt of Compound 1 is shown in FIG. 21.

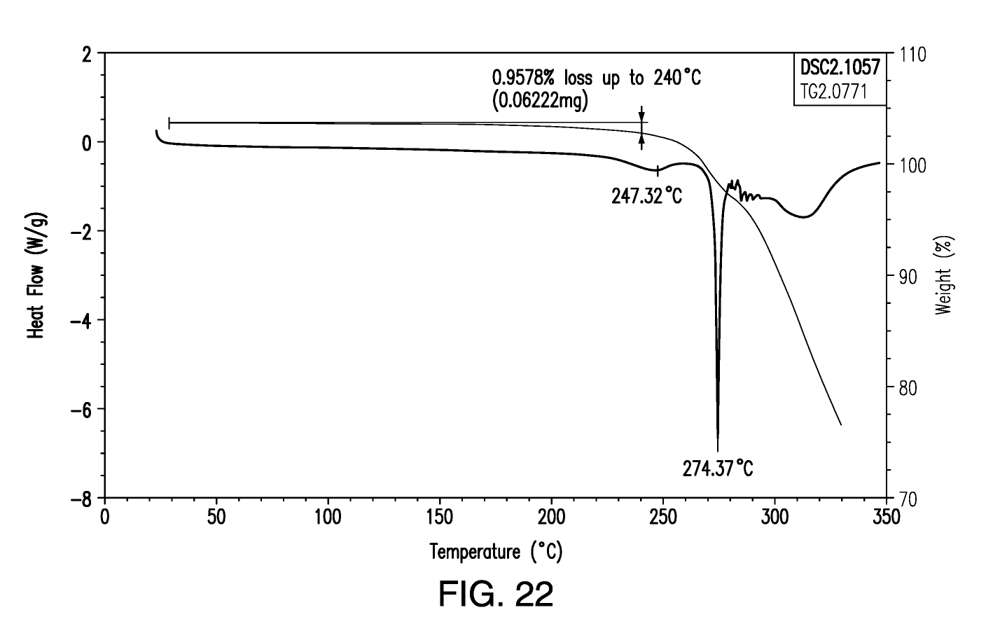

Representative thermal characteristics for Form A of the sulfate salt of Compound 1 are shown in FIG. 22. A representative TGA thermogram comprises a weight loss of about 0.96% based on the total weight of sample upon heating up to about 240° C. In certain embodiments, the aforementioned weight loss comprises a loss of solvent, such as, e.g., a loss of acetone.

In certain embodiments, Form A of the sulfate salt of Compound 1 is characterized by a DSC thermogram comprising one or more thermal events with the following approximate temperatures: 247 and 274° C. In certain embodiments, Form A of the sulfate salt of Compound 1 is characterized by a DSC thermogram comprising one or more thermal events with the following approximate temperatures: 247.32 and 274.37° C. A representative DSC thermogram is shown in FIG. 22.

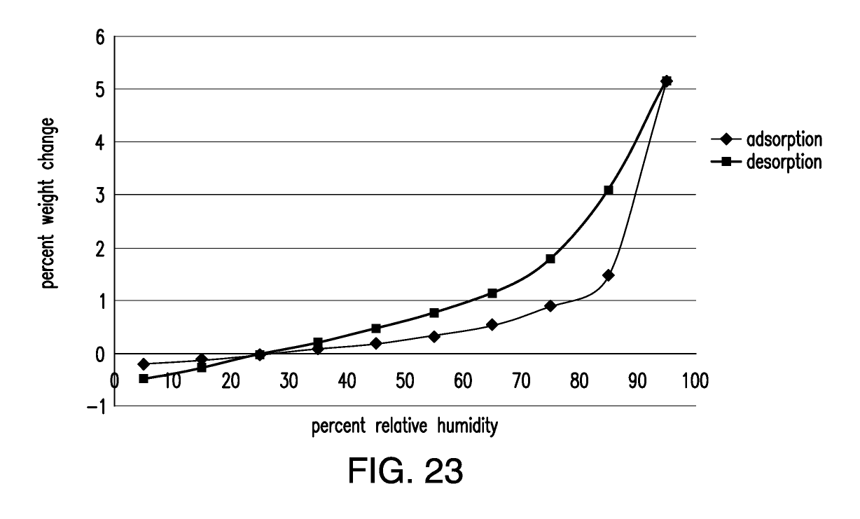

A representative dynamic vapor sorption (DVS) isotherm plot for Form A of the sulfate salt of Compound 1 is shown in FIG. 23. In certain embodiments, when the relative humidity (“RH”) is increased from about 5% to about 95% RH, Form A of the sulfate salt of Compound 1 exhibits about 5.36% weight gain. In certain embodiments, when the relative humidity (“RH”) is decreased from about 95% to about 5% RH, Form A of the sulfate salt of Compound 1 exhibits about 5.63% weight loss.

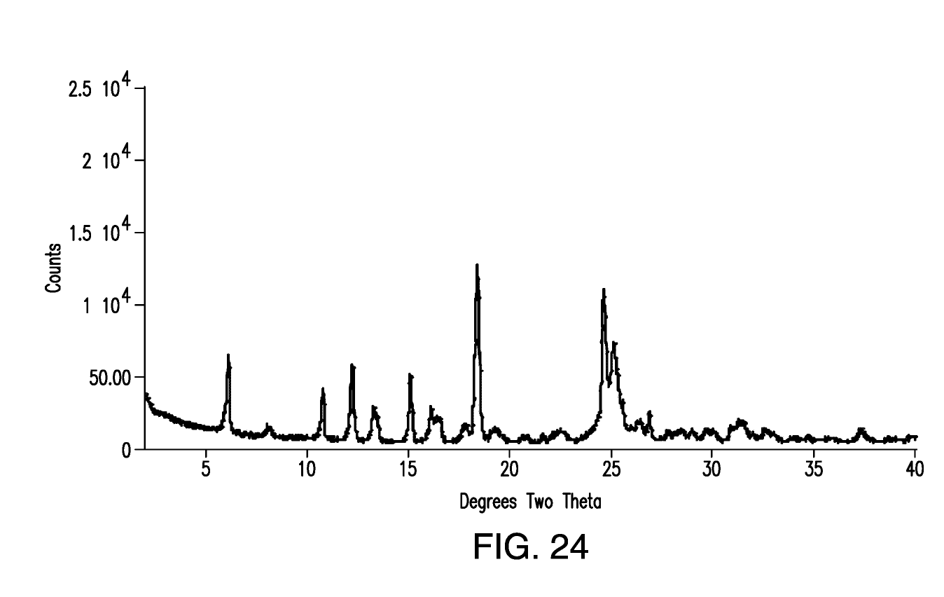

A representative XRPD pattern of Compound 1 sulfate salt Form A is unchanged post-dynamic vapor sorption as represented in FIG. 24.

In certain embodiments, the chemical profile of a sample of Form A of the sulfate salt of Compound 1 is characterized by solution NMR analysis. A representative ¹H NMR spectrum of a sample of Form A of the sulfate salt of Compound 1 dissolved in DMSO-d₆ is provided as FIGS. 25A and 25B

### 5.1.7. Thiocyanate Salt of Compound 1

Certain embodiments herein provide Form A and Form B of thiocyanate salt of Compound 1.

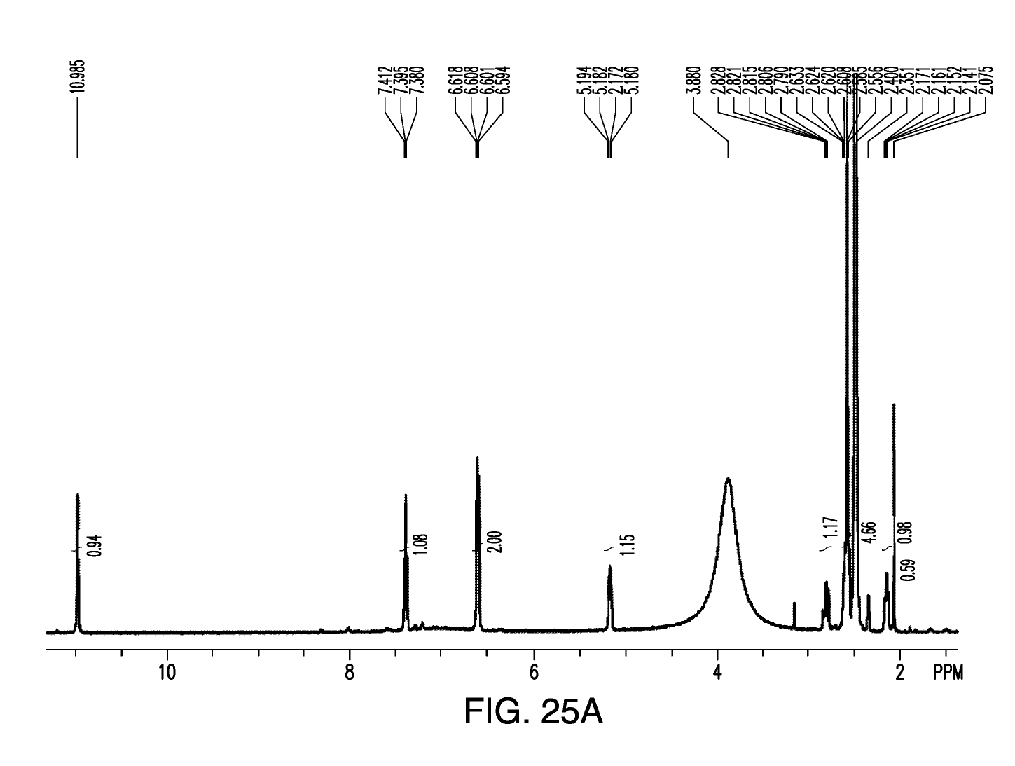

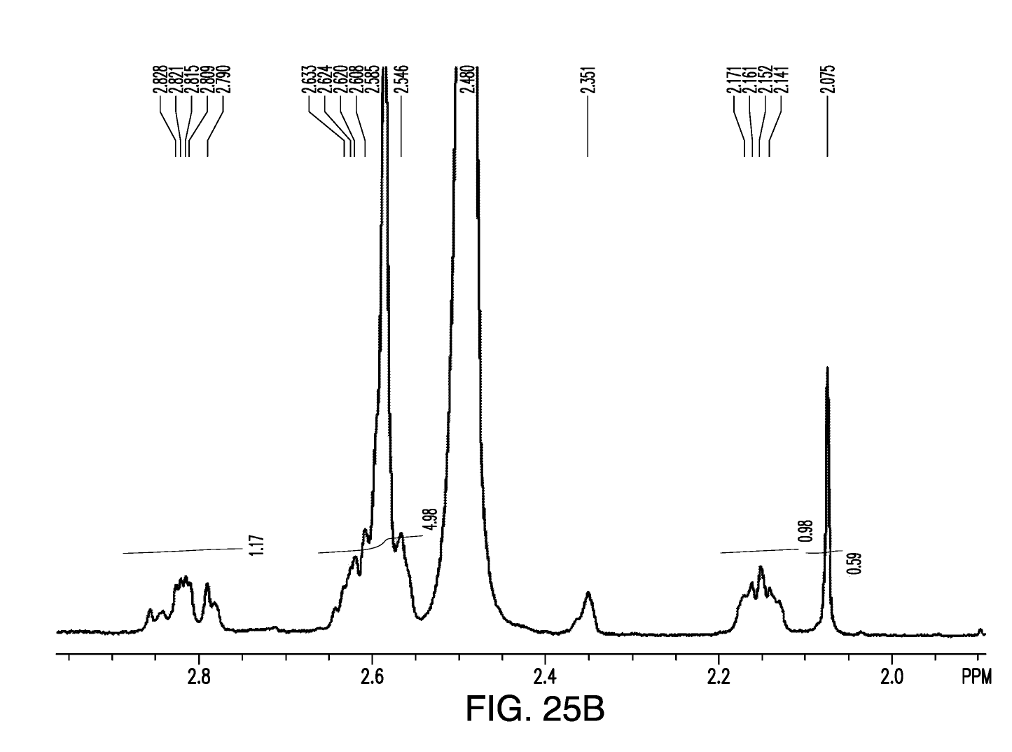

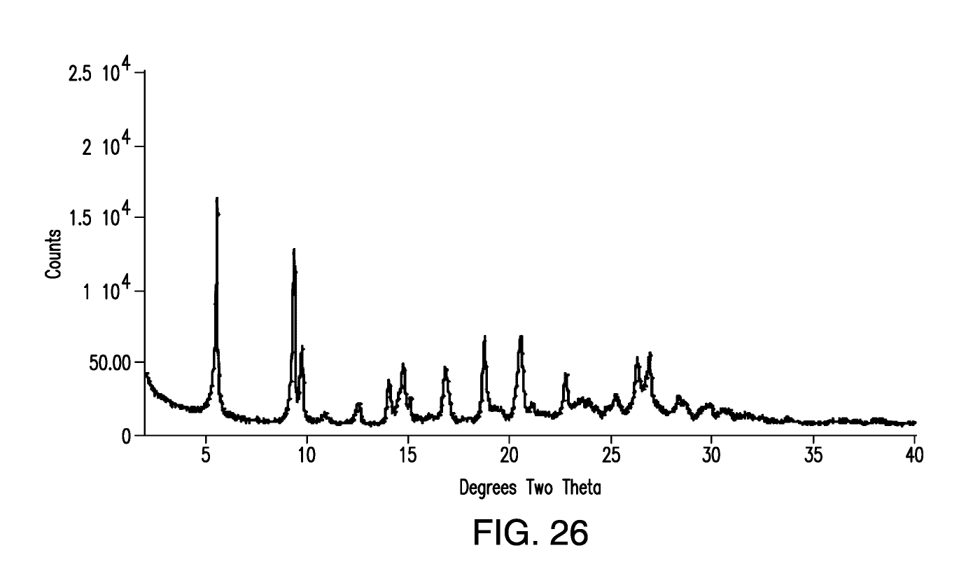

A representative XRPD pattern of Form A of the thiocyanate salt of Compound 1 is shown in FIG. 26.

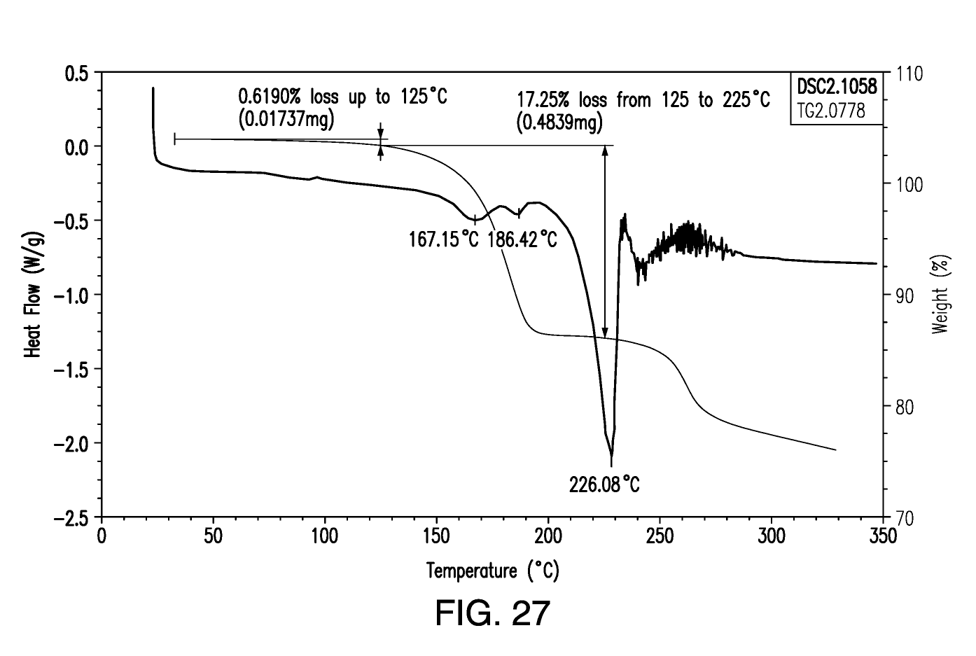

Representative thermal characteristics for Form A of the thiocyanate salt of Compound 1 are shown in FIG. 27. A representative TGA thermogram comprises a weight loss of about 0.62% based on the total weight of sample upon heating up to about 125° C., and weight loss of about 17.25% upon heating from 125° C. to about 175° C. In certain embodiments, the aforementioned weight loss of 17.25% comprises volatilization of approximately one mole of thiocyanic acid.

In certain embodiments, Form A of the thiocyanate salt of Compound 1 is characterized by a DSC thermogram comprising one or more thermal events with the following approximate temperatures: 167, 186, and 228° C. In certain embodiments, Form A of the thiocyanate salt of Compound 1 is characterized by a DSC thermogram comprising one or more thermal events with the following approximate temperatures: 167.18, 186.42, and 228.08° C. A representative DSC thermogram is shown in FIG. 27.

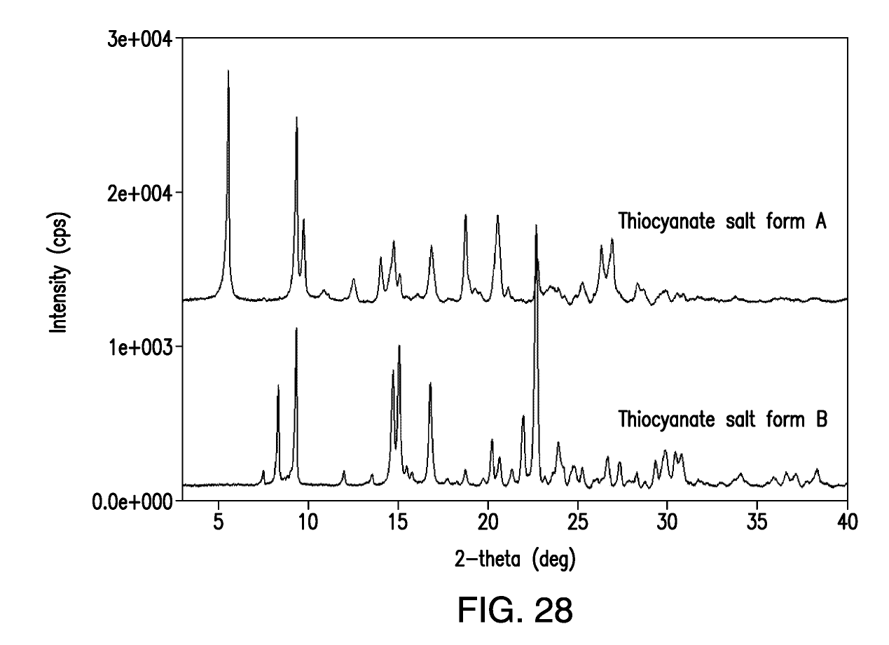

Representative XRPD patterns of Compound 1 thiocyanate salt Form A and Form B are overlaid and shown in FIG. 28.

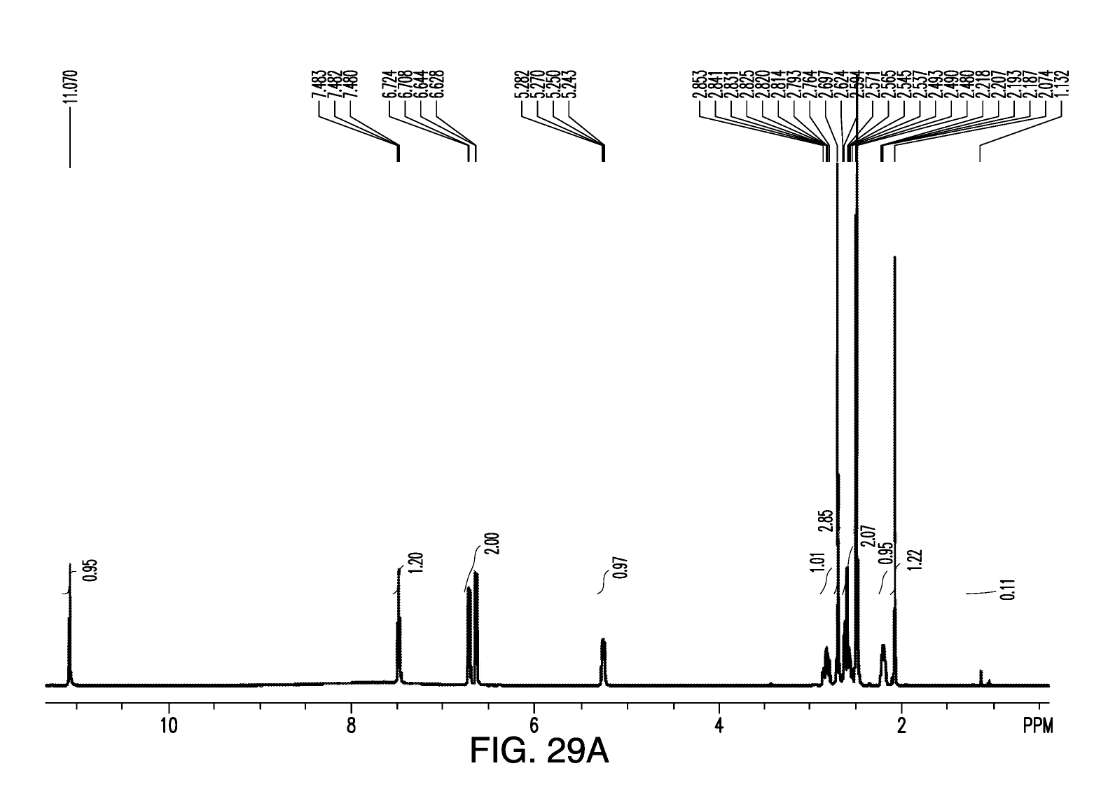

In certain embodiments, the chemical profile of a sample of Form A of the thiocyanate salt of Compound 1 is characterized by solution NMR analysis. A representative ¹H NMR spectrum of a sample of Form A of the thiocyanate salt of Compound 1 dissolved in DMSO-d₆ is provided as FIGS. 29A and 29B.

### 5.1.8. Tosylate Salt of Compound 1

Certain embodiments herein provide Form A and Form B of tosylate salt of Compound 1.

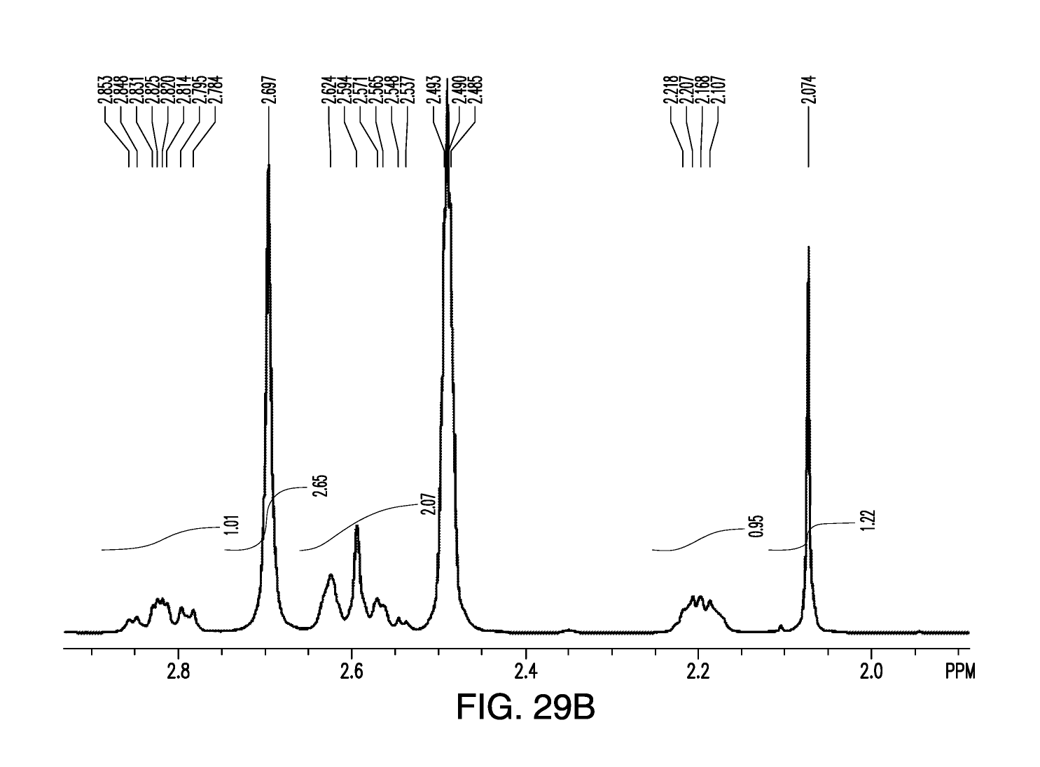

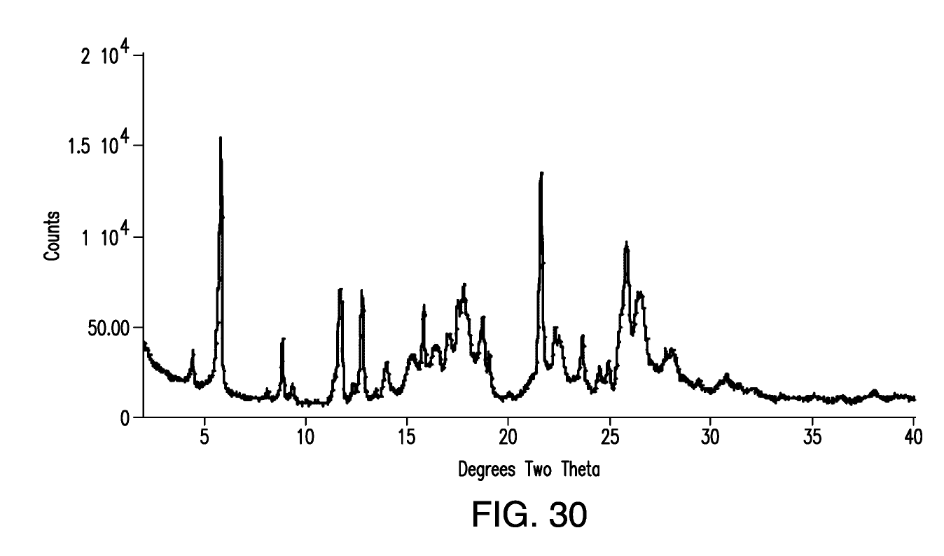

A representative XRPD pattern of Form A of the tosylate salt of Compound 1 is shown in FIG. 30.

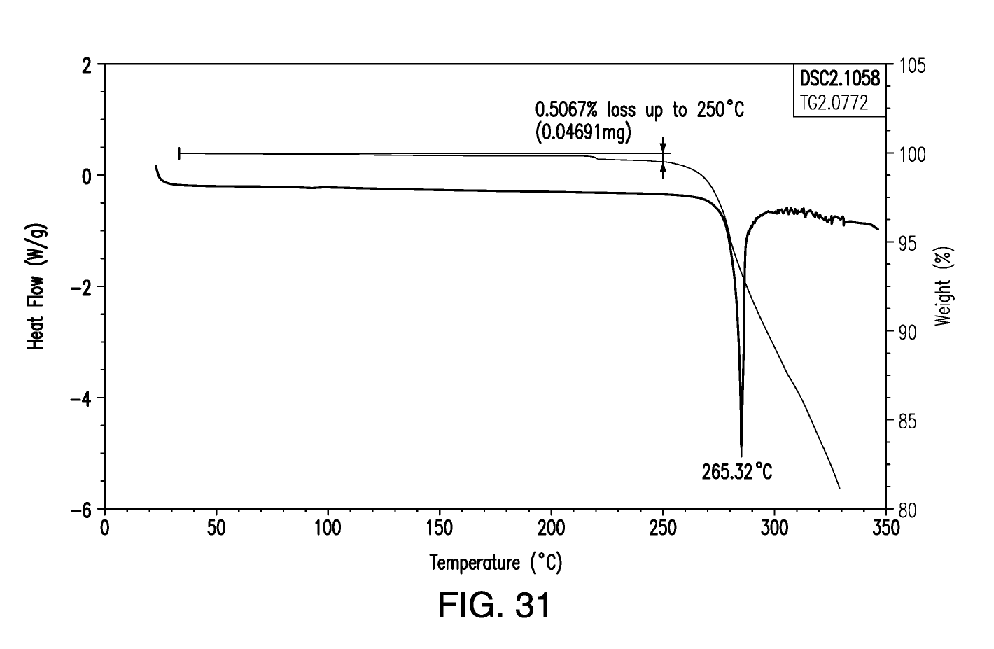

Representative thermal characteristics for Form A of the tosylate salt of Compound 1 are shown in FIG. 31. A representative TGA thermogram comprises a weight loss of about 0.51% based on the total weight of sample upon heating up to about 250° C. In certain embodiments, the aforementioned weight loss comprises a loss of solvent, such as, e.g., a loss of acetone.

In certain embodiments, Form A of the tosylate salt of Compound 1 is characterized by a DSC thermogram comprising a thermal event at approximate temperature of 285° C. In certain embodiments, Form A of the tosylate salt of Compound 1 is characterized by a DSC thermogram comprising a thermal event at approximate temperature of 285.32° C. A representative DSC thermogram is shown in FIG. 31.

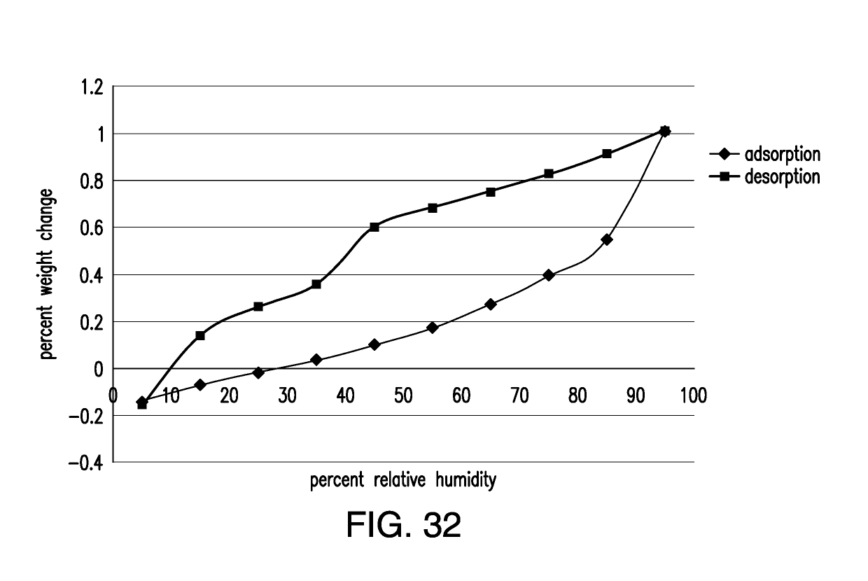

A representative dynamic vapor sorption (DVS) isotherm plot for Form A of the tosylate salt of Compound 1 is shown in FIG. 32. In certain embodiments, when the relative humidity (“RH”) is increased from about 5% to about 95% RH, Form A of the tosylate salt of Compound 1 exhibits about 1.15% weight gain. In certain embodiments, when the relative humidity (“RH”) is decreased from about 95% to about 5% RH, Form A of the tosylate salt of Compound 1 exhibits about 1.17% weight loss.

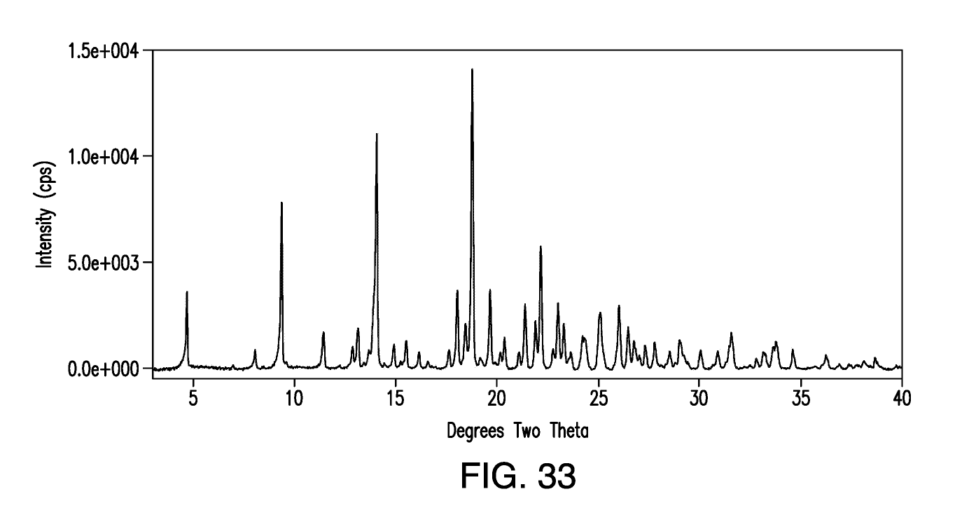

A representative XRPD pattern of Compound 1 tosylate salt Form B is represented in FIG. 33.

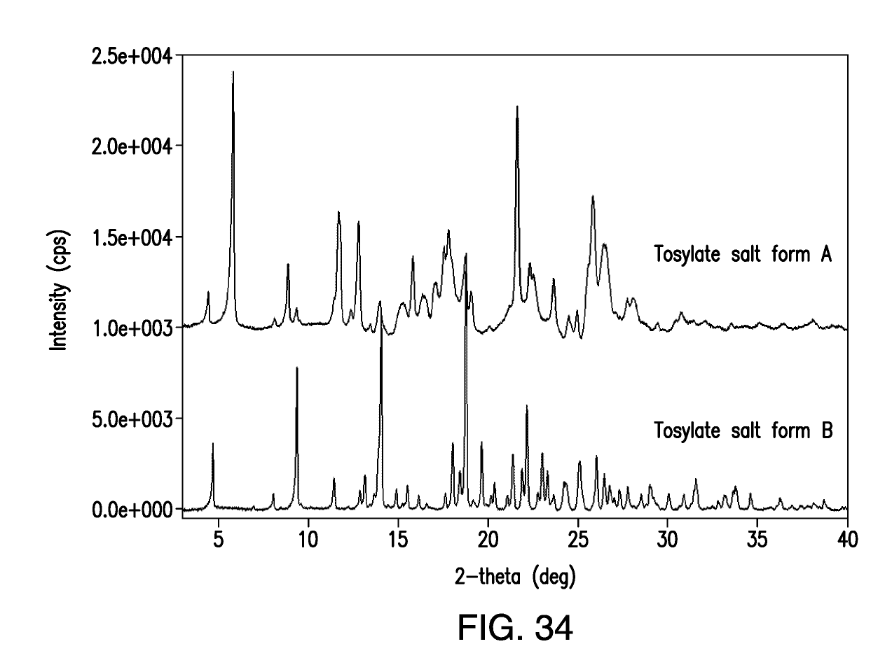

Representative XRPD patterns of Compound 1 tosylate salt Form A and Form B are overlaid and shown in FIG. 34.

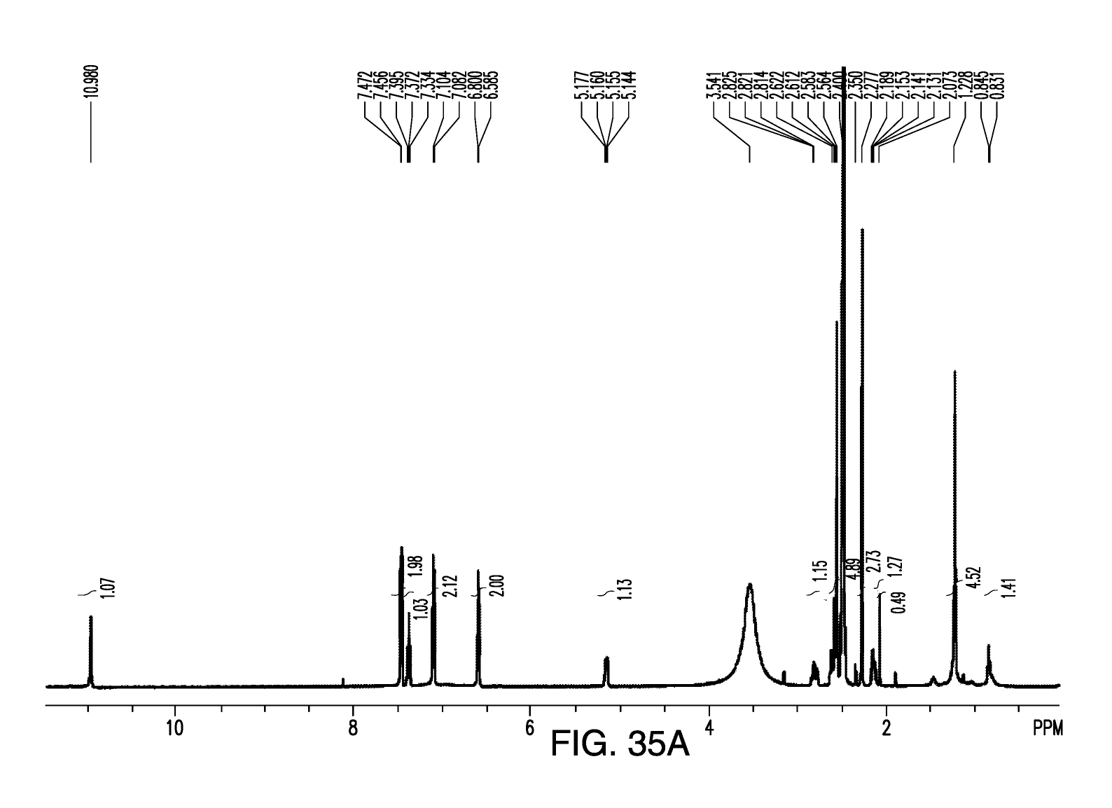

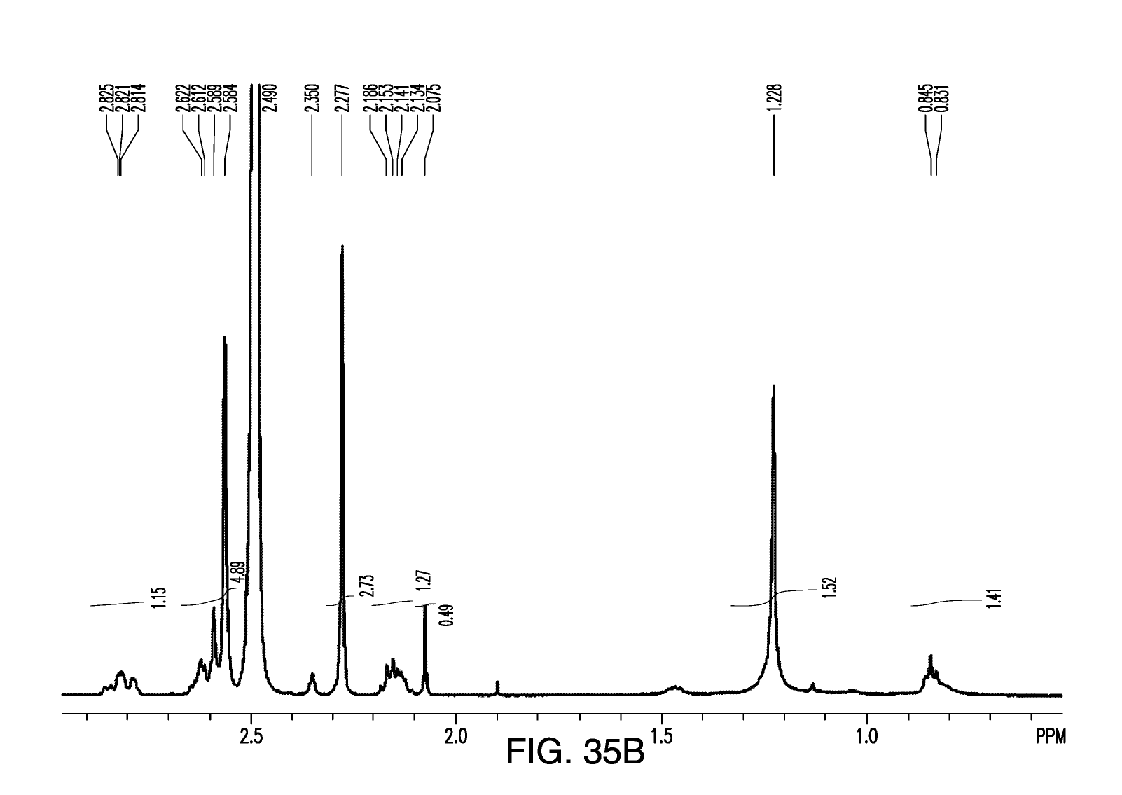

In certain embodiments, the chemical profile of a sample of Form A of the tosylate salt of Compound 1 is characterized by solution NMR analysis. A representative ¹H NMR spectrum of a sample of Form A of the tosylate salt of Compound 1 dissolved in DMSO-d₆ is provided as FIGS. 35A and 35B.

### 5.1.9. Compound 1 Acetic Acid Cocrystal

Certain embodiments herein provide Form A of Compound 1 acetic acid cocrystal. In one embodiment, Form A of Compound 1 acetic acid cocrystal provided herein is prepared by stoichiometric slurry of Compound 1 in acetic acid at room temperature.

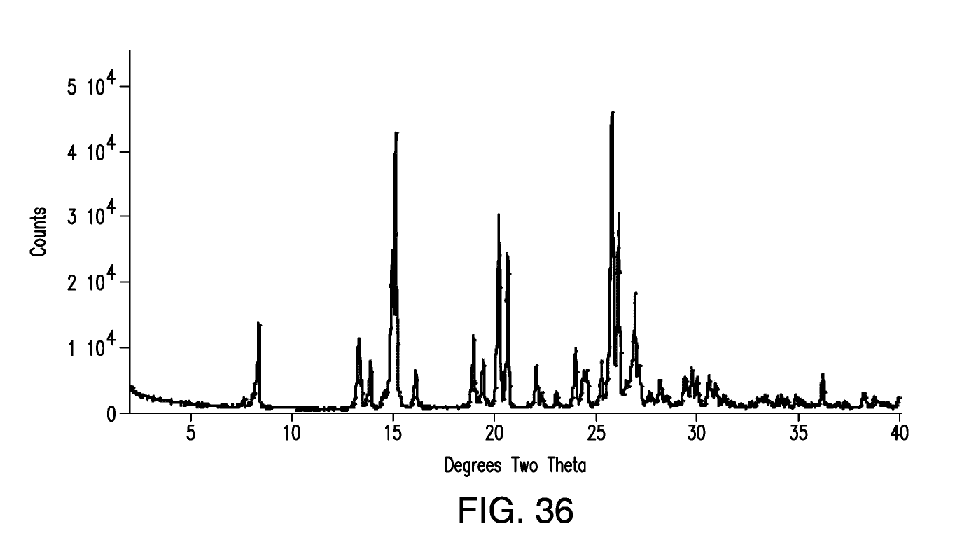

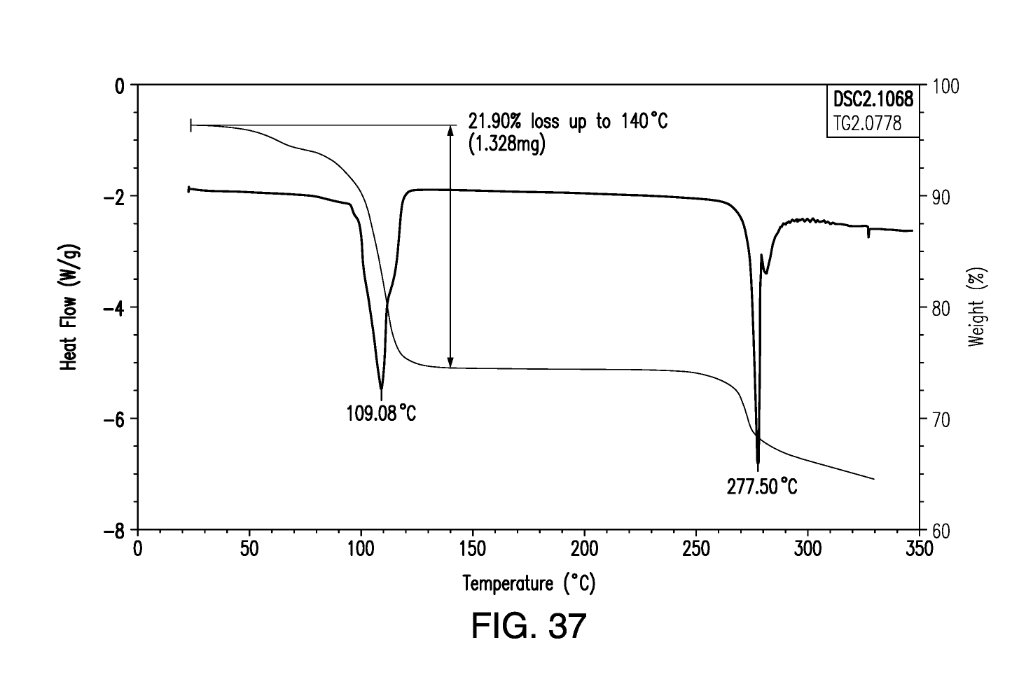

A representative XRPD pattern of Form A of Compound 1 acetic acid cocrystal is shown in FIG. 36. Representative thermal characteristics for Form A of Compound 1 acetic acid cocrystal are shown in FIG. 37. A representative TGA thermogram comprises a weight loss of about 21.90% based on the total weight of sample upon heating up to about 140° C. The aforementioned weight loss corresponds to 1.3 moles of acetic acid.

In certain embodiments, Form A of Compound 1 acetic acid cocrystal is characterized by a DSC thermogram comprising one or more thermal events with the following approximate temperatures: 109 and 278° C. In certain embodiments, Form A of Compound 1 acetic acid cocrystal is characterized by a DSC thermogram comprising one or more thermal events with the following approximate temperatures: 109.08 and 277.56° C. A representative DSC thermogram is shown in FIG. 37.

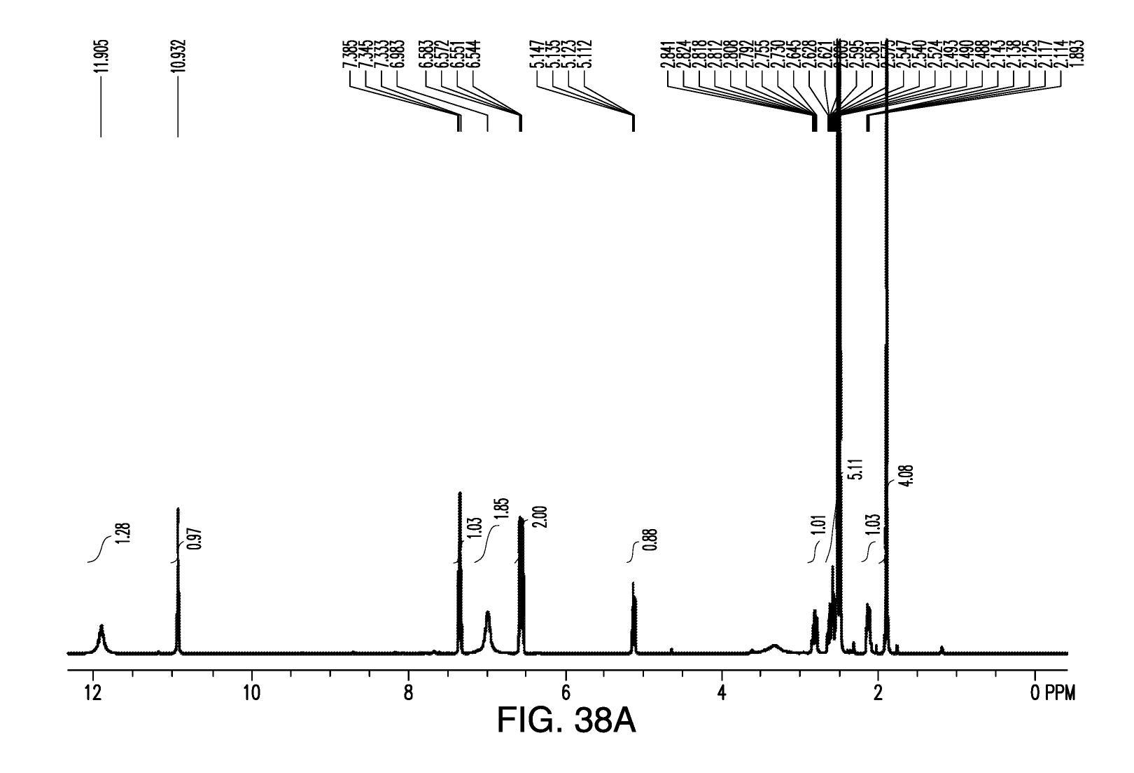

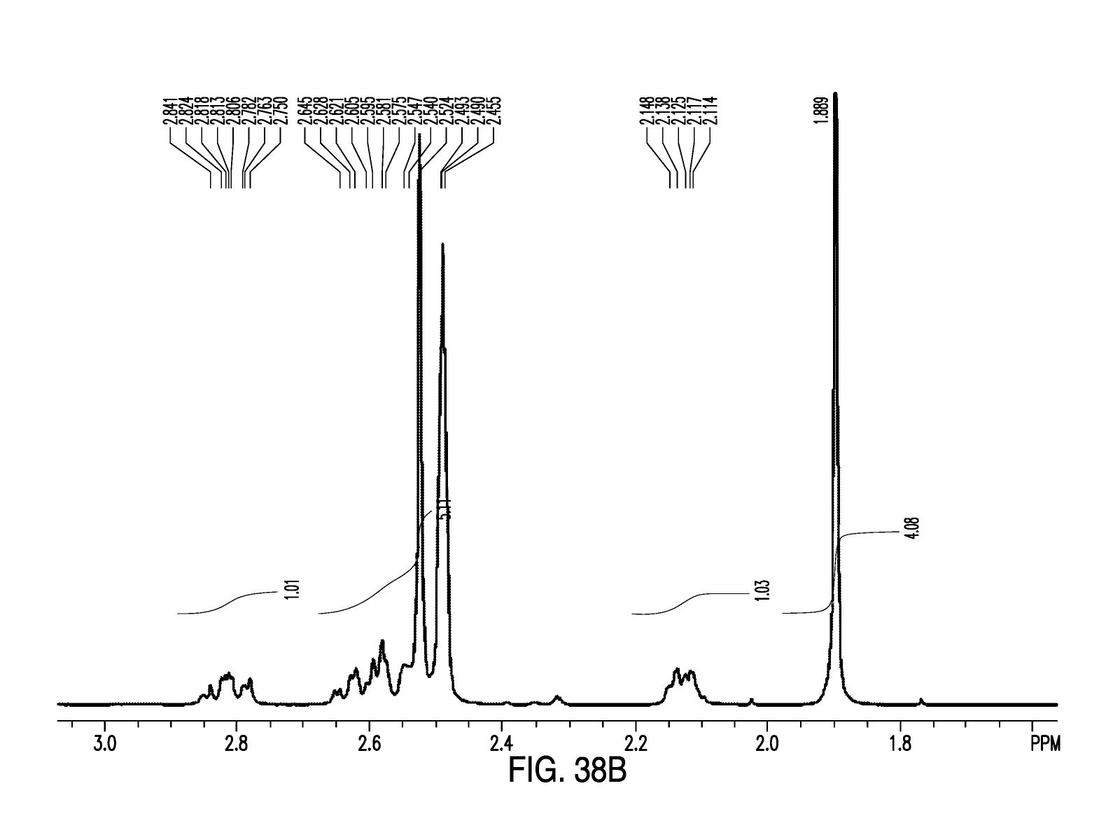

In certain embodiments, the chemical profile of a sample of Form A of Compound 1 acetic acid cocrystal is characterized by solution NMR analysis. A representative ¹H NMR spectrum of a sample of Form A of Compound 1 acetic acid cocrystal dissolved in DMSO-d₆ is provided as FIGS. 38A and 38B.

### 5.1.10. Compound 1 Isethionic Acid Cocrystal

Certain embodiments herein provide Form A and Form B of Compound 1 isethionic acid cocrystal. In one embodiment, Form B of Compound 1 isethionic acid cocrystal provided herein is prepared by stoichiometric slurry of Compound 1 and isethionic acid in acetone at room temperature.

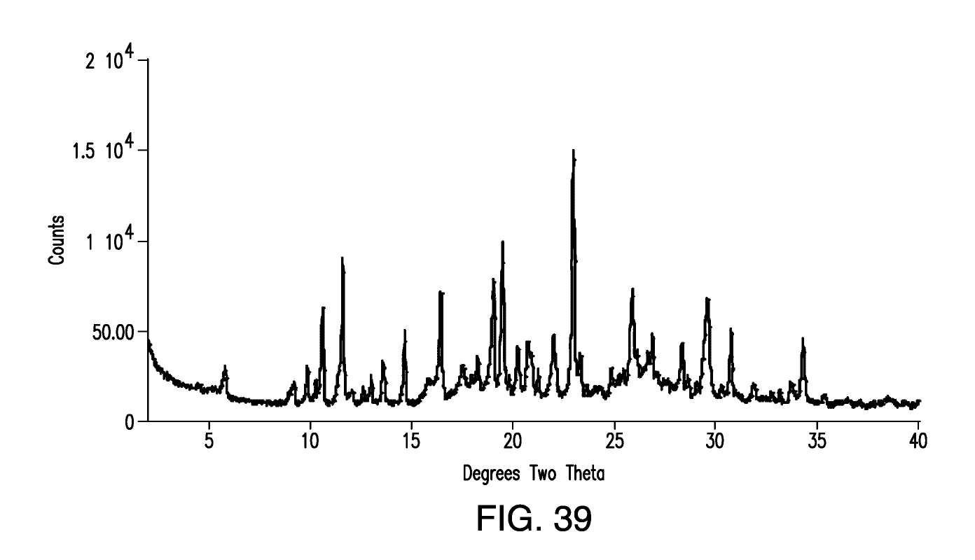

A representative XRPD pattern of Form B of Compound 1 isethionic acid cocrystal is shown in FIG. 39.

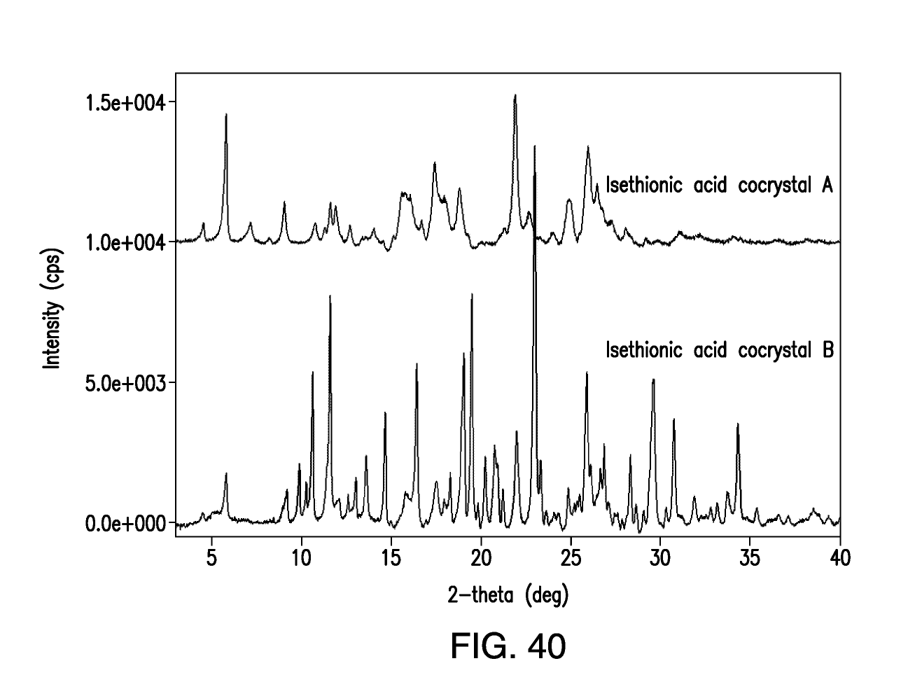

Representative XRPD patterns of Compound 1 isethionic cocrystal Form A and Form B are overlaid and shown in FIG. 40.

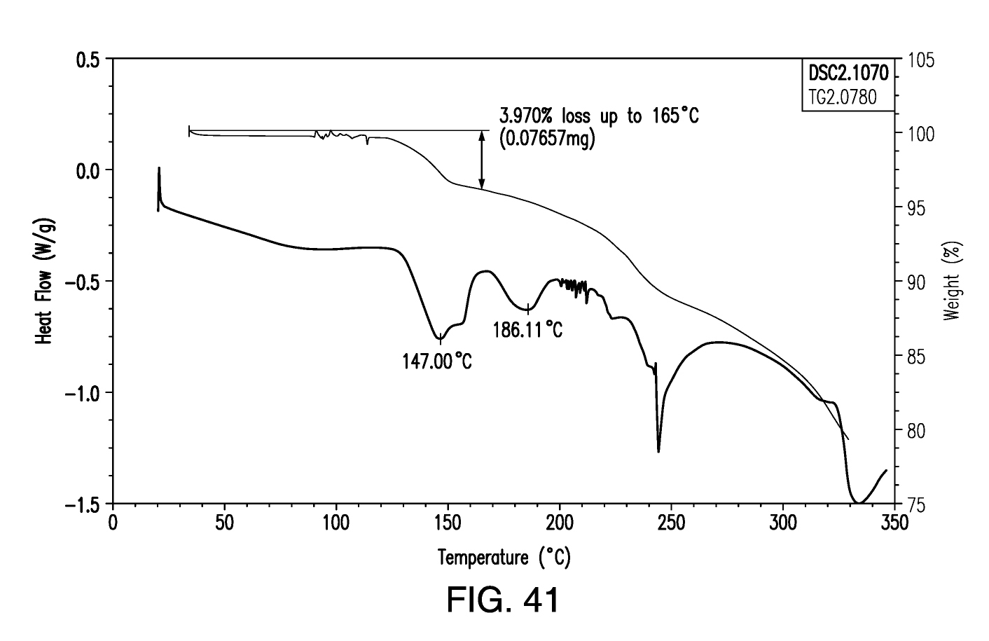

Representative thermal characteristics for Compound 1 isethionic acid cocrystal form B are shown in FIG. 41. A representative TGA thermogram comprises a weight loss of about 4.0% based on the total weight of sample upon heating up to about 165° C. The aforementioned weight loss corresponds to 0.21 moles of acetone.

In certain embodiments, Compound 1 isethionic acid cocrystal Form B is characterized by a DSC thermogram comprising one or more thermal events with the following approximate temperatures: 147 and 186° C. In certain embodiments, Compound 1 isethionic acid cocrystal Form B is characterized by a DSC thermogram comprising one or more thermal events with the following approximate temperatures: 147.00 and 186.11° C. A representative DSC thermogram is shown in FIG. 41.

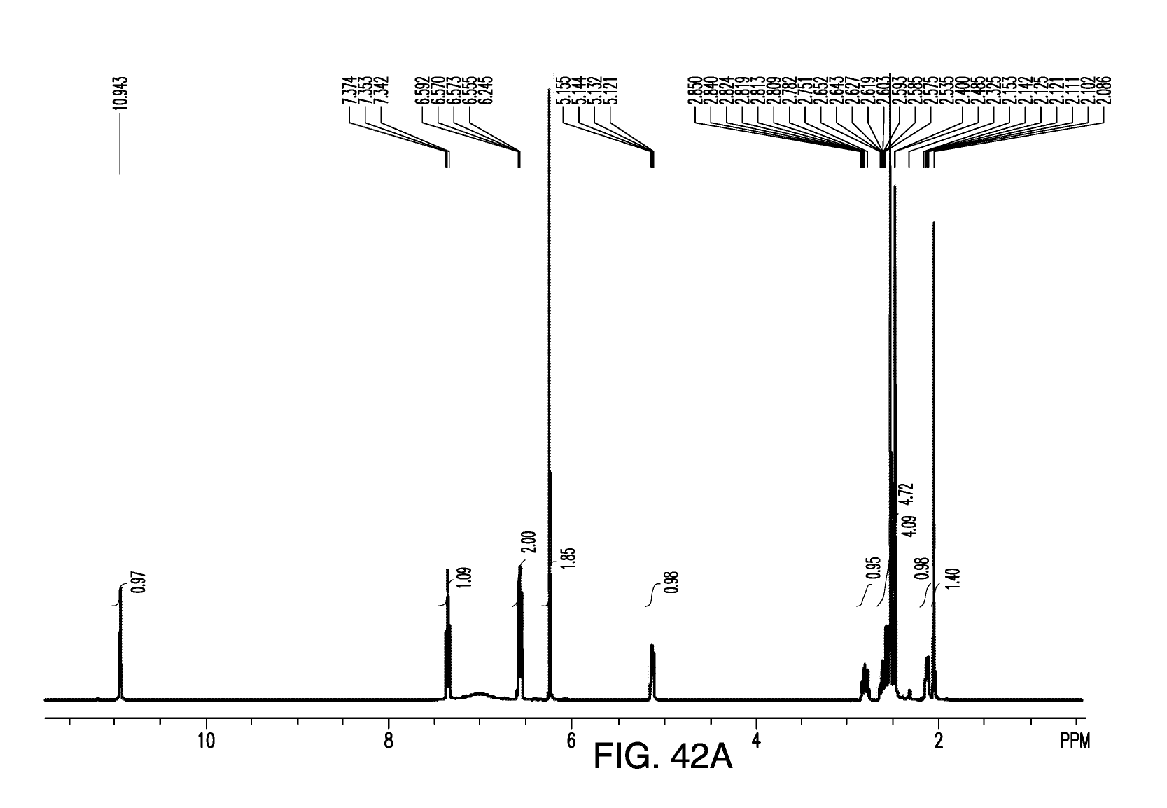

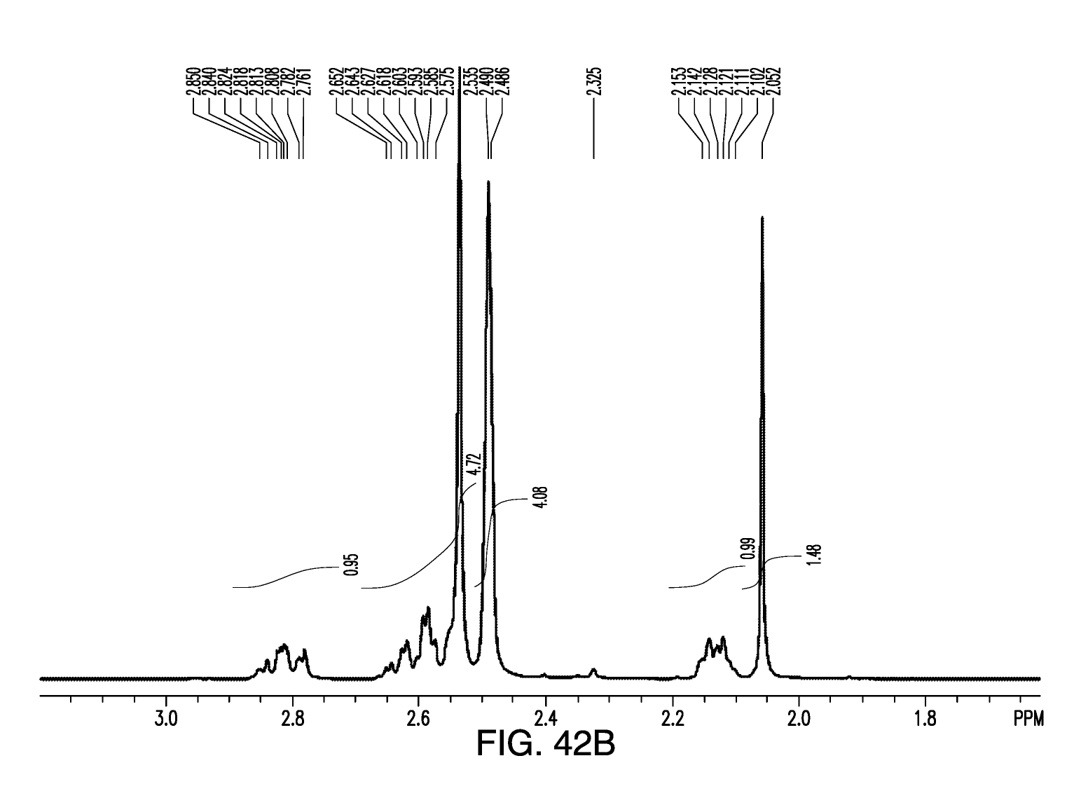

In certain embodiments, the chemical profile of a sample of Compound 1 isethionic acid cocrystal Form B is characterized by solution NMR analysis. A representative ¹H NMR spectrum of a sample of Compound 1 isethionic acid cocrystal Form B dissolved in DMSO-d₆ is provided as FIGS. 42A and 42B.

### 5.1.11. Compound 1 Lauric Acid Cocrystal

Certain embodiments herein provide Form A of Compound 1 lauric acid cocrystal. In one embodiment, Form A of Compound 1 lauric acid cocrystal provided herein is prepared by stoichiometric slurry of Compound 1 and lauric acid in acetone at room temperature.

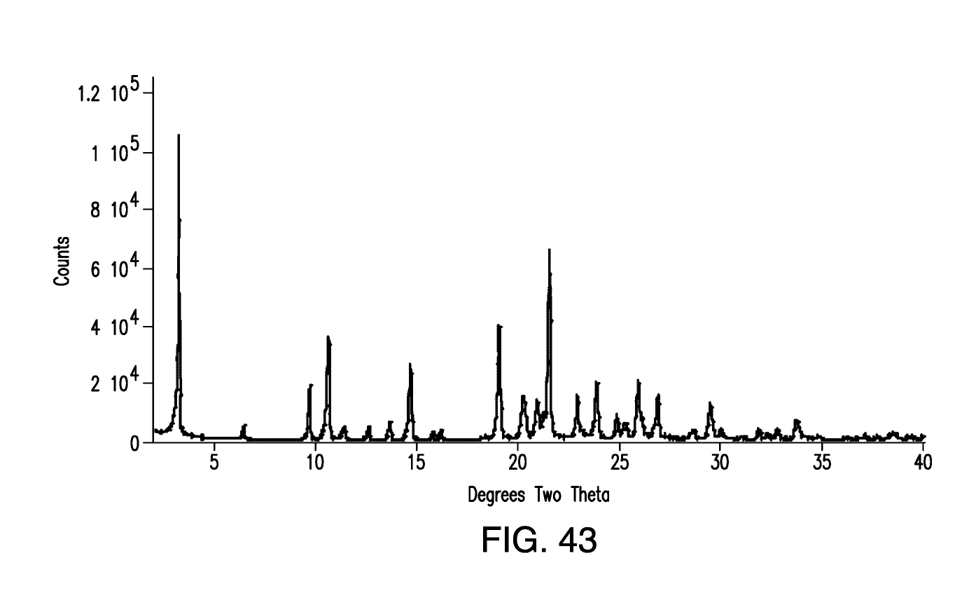

A representative XRPD pattern of Form A of Compound 1 lauric acid cocrystal is shown in FIG. 43.

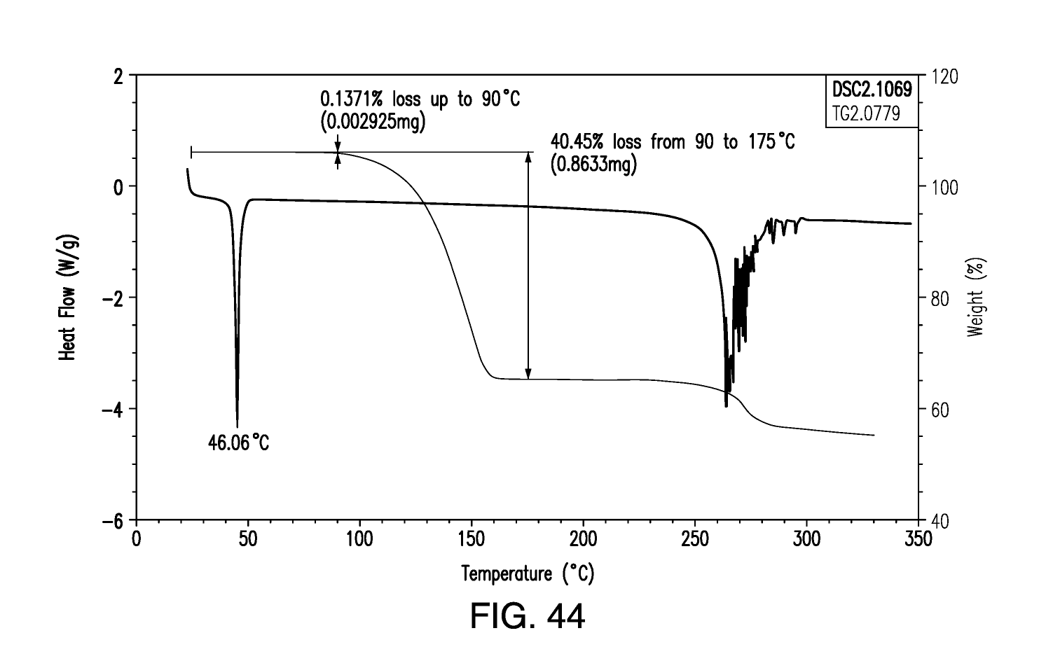

Representative thermal characteristics for Form A of Compound 1 lauric acid cocrystal are shown in FIG. 44. A representative TGA thermogram comprises a weight loss of about 0.14% based on the total weight of sample upon heating up to about 90° C. The aforementioned weight loss comprises a loss of solvent, such as, e.g., a loss of acetone.

In certain embodiments, Form A of Compound 1 lauric acid cocrystal is characterized by a DSC thermogram comprising a thermal event at approximate temperature of 45° C. In certain embodiments, Form A of Compound 1 lauric acid cocrystal is characterized by a DSC thermogram comprising a thermal event at approximate temperature of 45.05° C. A representative DSC thermogram is shown in FIG. 44.

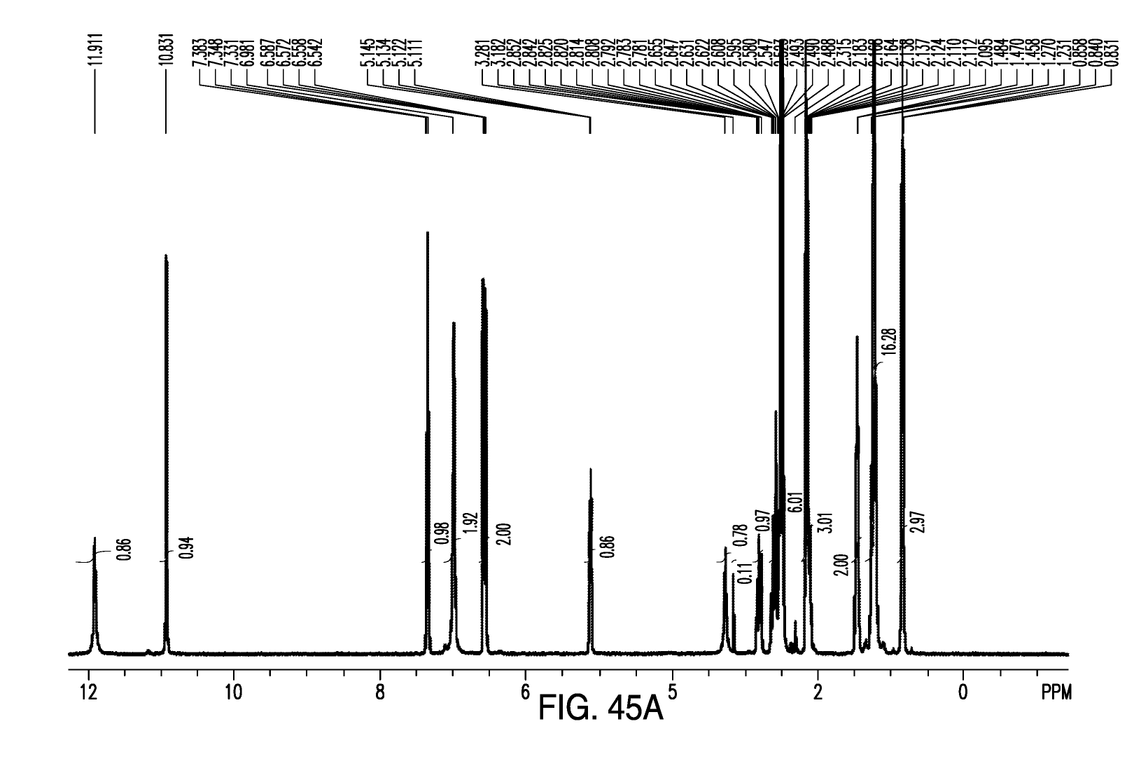

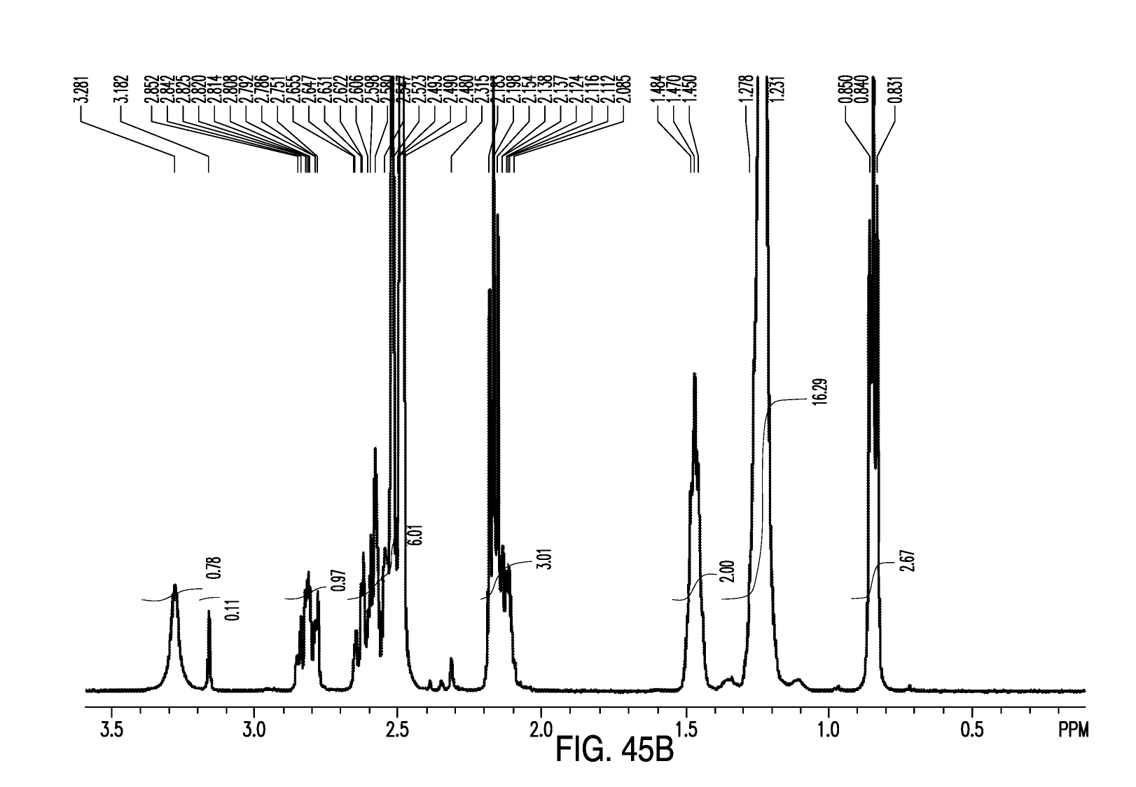

In certain embodiments, the chemical profile of a sample of Form A of Compound 1 lauric acid cocrystal is characterized by solution NMR analysis. A representative ¹H NMR spectrum of a sample of Form A of Compound 1 lauric acid cocrystal dissolved in DMSO-d₆ is provided as FIGS. 45A and 45B.

### 5.1.12. Compound 1 Maleic Acid Cocrystal

Certain embodiments herein provide Form A of Compound 1 maleic acid cocrystal. In one embodiment, Form A of Compound 1 maleic acid cocrystal provided herein is prepared by stoichiometric slurry of Compound 1 and maleic acid in acetonitrile at room temperature.

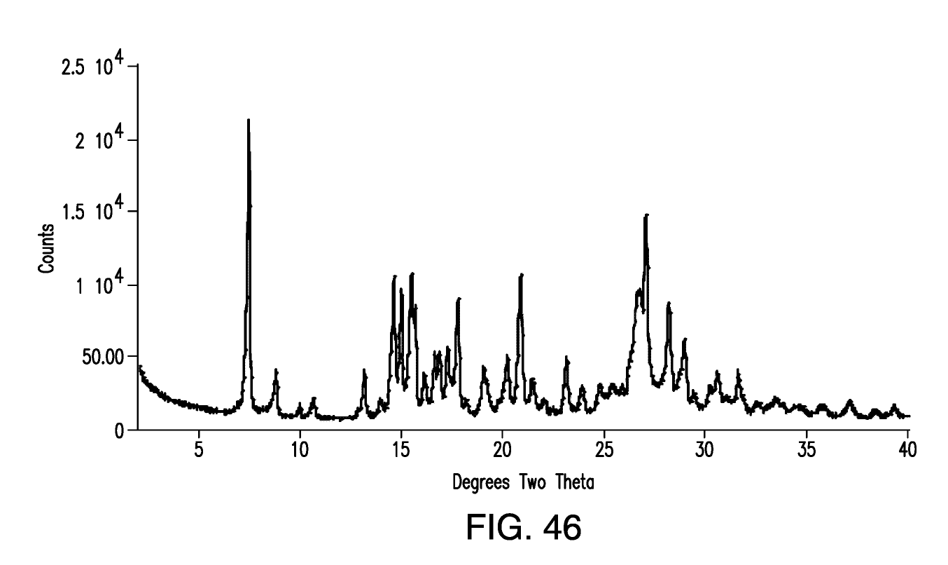

A representative XRPD pattern of Form A of Compound 1 maleic acid cocrystal is shown in FIG. 46.

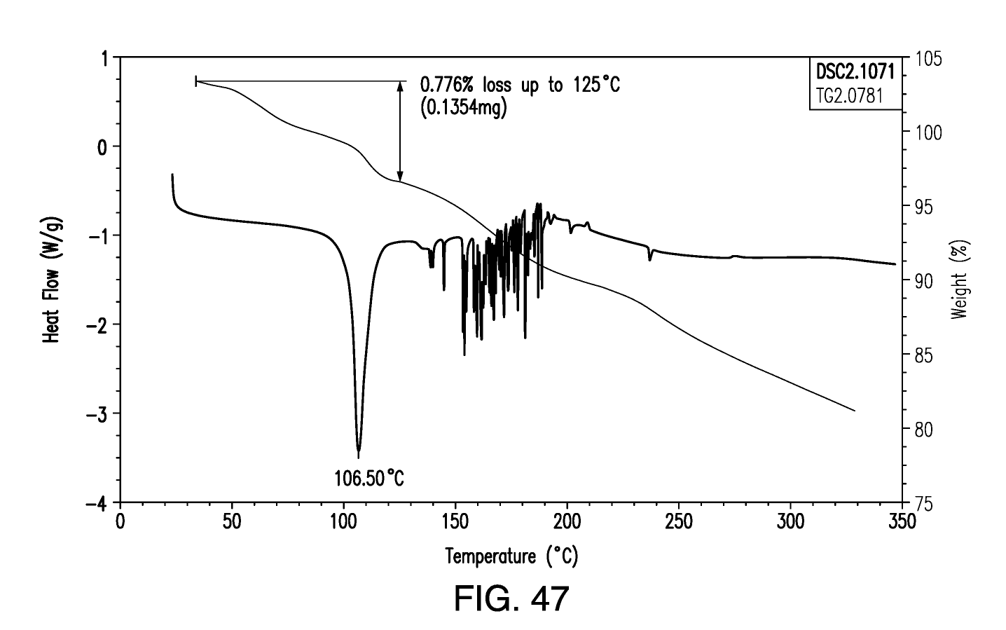

Representative thermal characteristics for Form A of Compound 1 maleic acid cocrystal are shown in FIG. 47. A representative TGA thermogram comprises a weight loss of about 6.8% based on the total weight of sample upon heating up to about 125° C. The aforementioned weight loss comprises a loss of solvent, such as, e.g., a loss of acetonitrile.

In certain embodiments, Form A of Compound 1 maleic acid cocrystal is characterized by a DSC thermogram comprising a thermal event at approximate temperature of 106° C. In certain embodiments, Form A of Compound 1 maleic acid cocrystal is characterized by a DSC thermogram comprising a thermal event at approximate temperature of 106.50° C. A representative DSC thermogram is shown in FIG. 47.

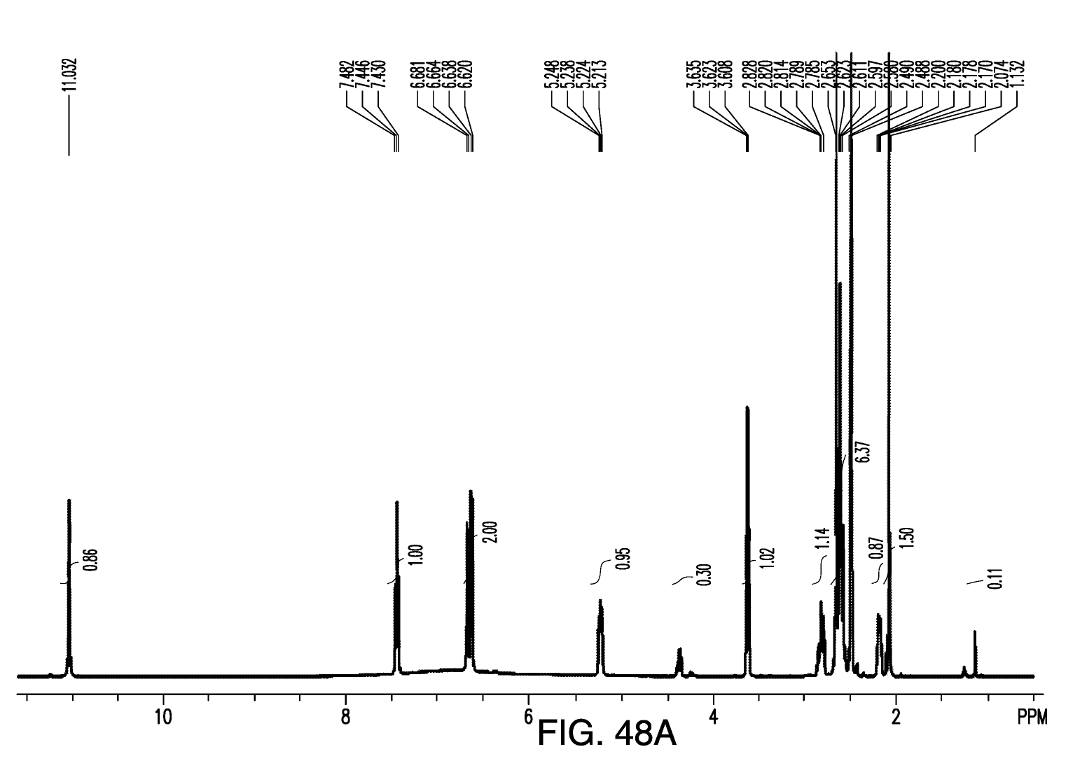

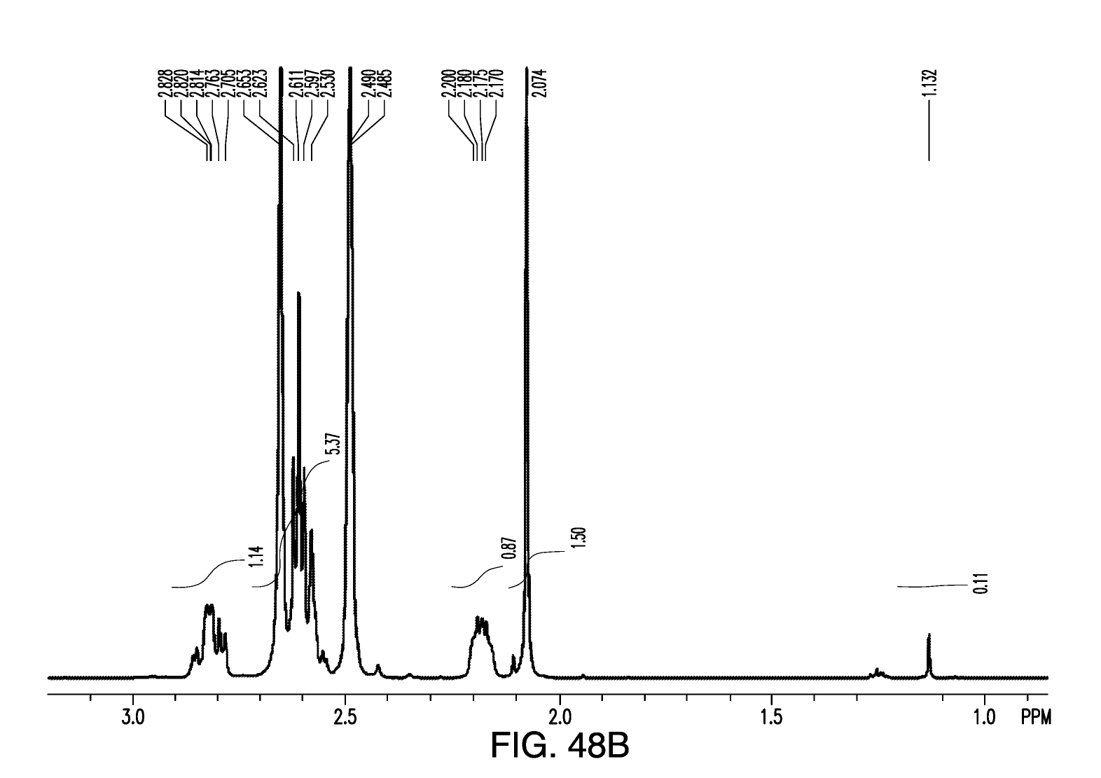

In certain embodiments, the chemical profile of a sample of Form A of Compound 1 maleic acid cocrystal is characterized by solution NMR analysis. A representative ¹H NMR spectrum of a sample of Form A of Compound 1 maleic acid cocrystal dissolved in DMSO-d₆ is provided as FIGS. 48A and 48B.

### 5.1.13. Compound 1 Phosphoric Acid Cocrystal

Certain embodiments herein provide Form A of Compound 1 phosphoric acid cocrystal. In one embodiment, Form A of Compound 1 phosphoric acid cocrystal provided herein is prepared by stoichiometric precipitation of Compound 1 and phosphoric acid in acetone at room temperature. In certain embodiments, Form A of Compound 1 phosphoric acid cocrystal is solvated.

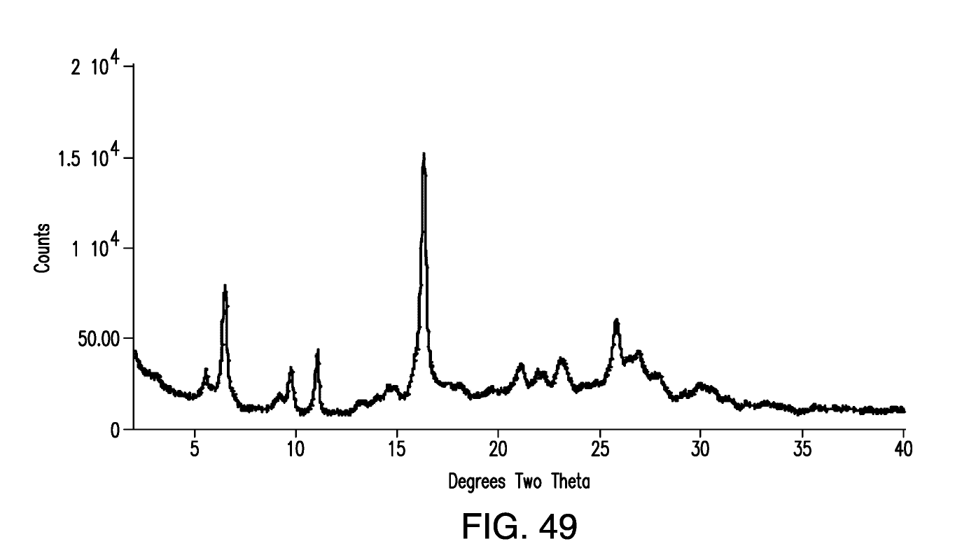

A representative XRPD pattern of Form A of Compound 1 phosphoric acid cocrystal is shown in FIG. 49.

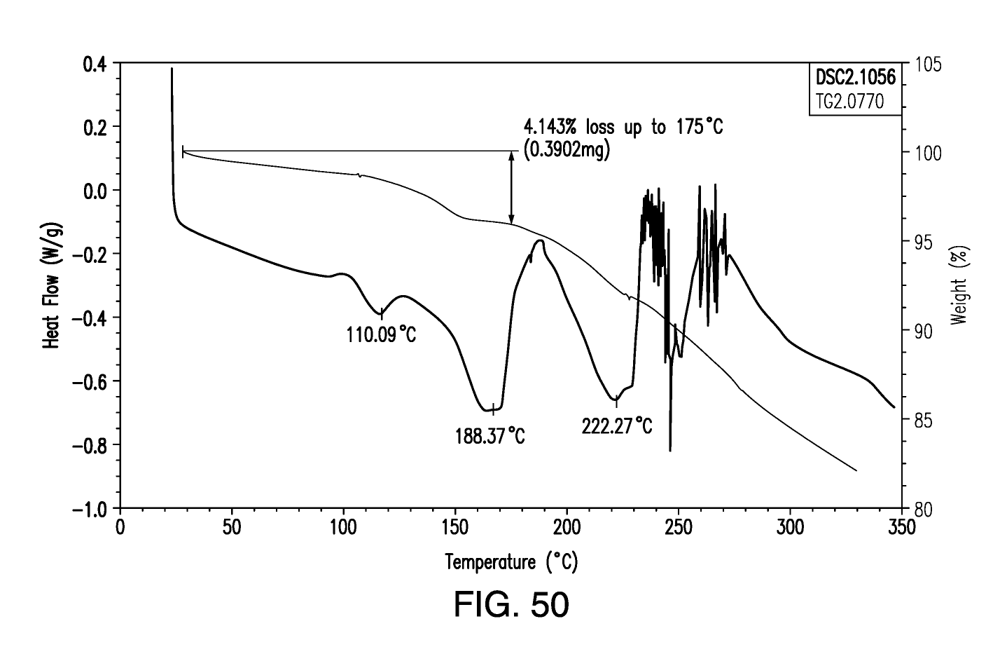

Representative thermal characteristics for Form A of Compound 1 phosphoric acid cocrystal are shown in FIG. 50. A representative TGA thermogram comprises a weight loss of about 4.1% based on the total weight of sample upon heating up to about 175° C. The aforementioned weight loss comprises a loss of solvent, such as, e.g., a loss of acetone.

In certain embodiments, Form A of Compound 1 phosphoric acid cocrystal is characterized by a DSC thermogram comprising one or more thermal events with the following approximate temperatures: 117, 166 and 222° C. In certain embodiments, Form A of Compound 1 phosphoric acid cocrystal is characterized by a DSC thermogram comprising one or more thermal events with the following approximate temperatures: 116.89, 166.37 and 222.27° C. A representative DSC thermogram is shown in FIG. 50.

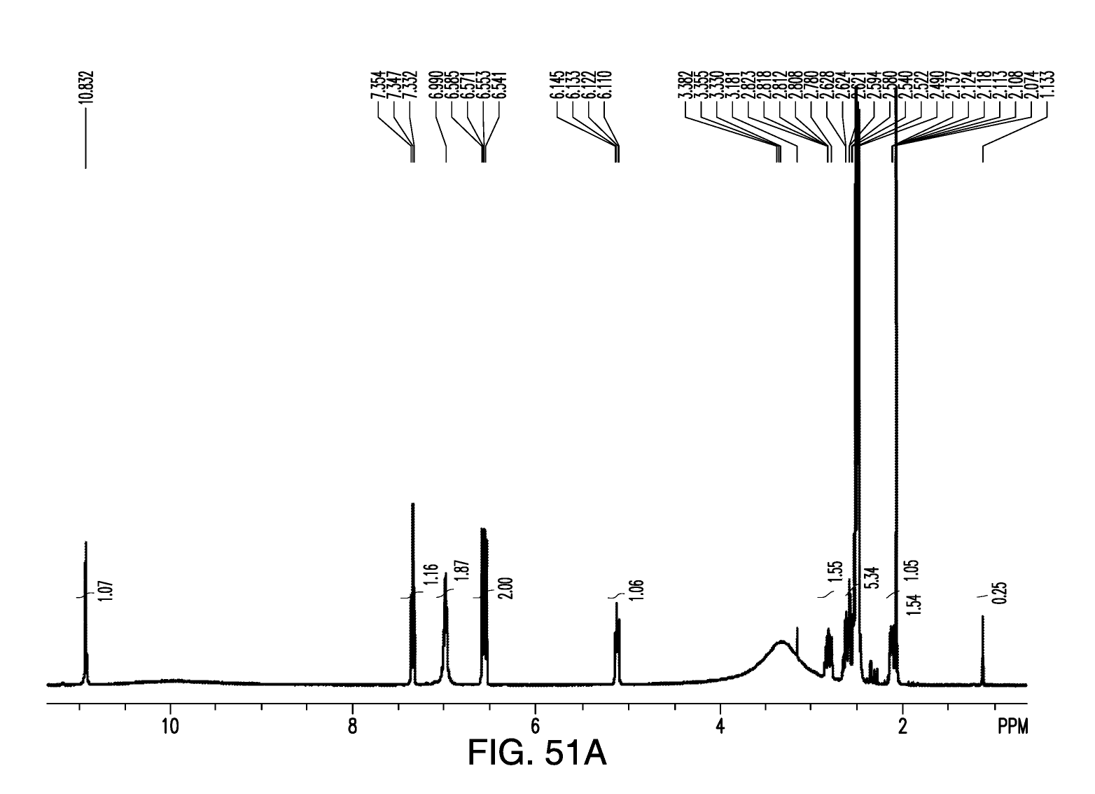

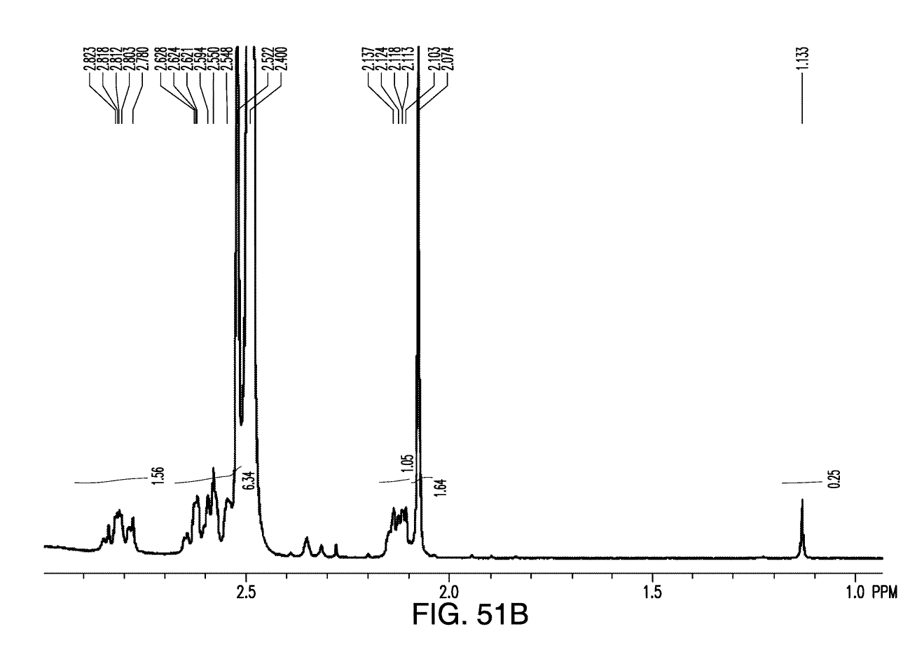

In certain embodiments, the chemical profile of a sample of Form A of Compound 1 phosphoric acid cocrystal is characterized by solution NMR analysis. A representative ¹H NMR spectrum of a sample of Form A of Compound 1 phosphoric acid cocrystal dissolved in DMSO-d₆ is provided as FIGS. 51A and 51B.

In certain embodiments, a solid form provided herein is characterized by a combination of the aforementioned characteristics of any one of the single crystalline forms discussed herein. The characterization may be by any combination of one or more of the XRPD, TGA, DSC, and DVS described for a particular polymorph. For example, a single crystalline form may be characterized by any combination of the XRPD results regarding the position of the major peaks in a XRPD scan; and/or any combination of one or more of parameters derived from data obtained from a XRPD scan. The single crystalline form provided herein may also be characterized by TGA determinations of the weight loss associated with a sample over a designated temperature range; and/or the temperature at which a particular weight loss transition begins. DSC determinations of the temperature associated with the maximum heat flow during a heat flow transition and/or the temperature at which a sample begins to undergo a heat flow transition may also characterize the crystalline form. Weight change in a sample and/or change in sorption/desorption of water per molecule of a solid form as determined by water sorption/desorption measurements over a range of relative humidity (e.g., 0% to 95%) may also characterize a single crystalline form provided herein.

### 5.2. SOLID FORM COMPRISING COMPOUND 1 OR COCRYSTAL COMPRISING COMPOUND 1 FOR USE IN METHODS OF TREATMENT

A solid form of a pharmaceutically acceptable salt of Compound 1 or a cocrystal comprising Compound 1 provided herein can be used in all methods of treatment provided herein.

The disclosure encompasses methods of treating, preventing, and/or managing various diseases or disorders using a solid form comprising Compound 1 provided herein, which comprise administering a therapeutically or prophylactically effective amount of one or more solid forms comprising Compound 1, or a pharmaceutically acceptable salt or cocrystal thereof such as, e.g., Form A of a sulfate salt of Compound 1, Form A or Form B of a mesylate salt of Compound 1, Form A or Form B of an esylate salt of Compound 1, Form A of a besylate salt of Compound 1, Form A of a napadisylate salt of Compound 1, Form A of a napsilate salt of Compound 1, Form A or Form B of a thiocyanate salt of Compound 1, and Form A or Form B of a tosylate salt of Compound 1; Compound 1 acetic acid cocrystal Form A, Compound 1 isethionic acid cocrystal Form A, Compound 1 isethionic acid cocrystal Form B, Compound 1 lauric acid cocrystal Form A, Compound 1 maleic acid cocrystal Form A, Compound 1 phosphoric acid cocrystal Form A as provided herein.

Without being limited by a particular theory, Compound 1 can control angiogenesis or inhibit the production of certain cytokines including, but not limited to, TNF-α, IL-1β, IL-18, GM-CSF, and/or IL-6. Without being limited by a particular theory, Compound 1 can stimulate the production of certain other cytokines including IL-10, and also act as a costimulatory signal for T cell activation, resulting in increased production of cytokines such as, but not limited to, IL-12 and/or IFN-γ. In addition, Compound 1 can enhance the effects of NK cells and antibody-mediated cellular cytotoxicity (ADCC). Further, Compound 1 may be immunomodulatory and/or cytotoxic, and thus, may be useful as chemotherapeutic agents. Consequently, without being limited by a particular theory, some or all of such characteristics possessed by Compound 1 may render them useful in treating, managing, and/or preventing various diseases or disorders.

Examples of hematological and solid cancers and precancerous conditions include, but are not limited to, cancers of the skin, such as melanoma; lymph node; breast; cervix; uterus; gastrointestinal tract; lung; including small cell and non-small cell lung cancer, ovary; prostate; colon; colorectal, bladder, rectum; mouth; head or neck; brain; head and neck; throat; testes; kidney; pancreas; renal cancer, thyroid, bone; spleen; liver; hepatocellular cancer; bladder; larynx; nasal passages kaposi's sacorma and AIDS-related cancers. The compounds are also useful for treating cancers of the blood and bone marrow, such as multiple myeloma and acute and chronic leukemias, for example, lymphoblastic, myelogenous, lymphocytic, myelocytic leukemias hodgkins lymphoma and Non-Hodgkins lymphoma. These coumpounds may also be useful for the treatment of Myleodysplastic Syndrome and Myeloproliferative Diseases. The compounds provided herein can be used for treating, preventing or managing either primary or metastatic tumors.

In certain embodiments, cancer is multiple myeloma, AML, CLL, Hodgkins lymphoma, non-Hodgkins lymphoma (NHL), including aggressive and indolent subtypes, including: cutaneous t cell lymphomas, follicular, mantle cell , Waldestrom macrogllobineima, plasmacytoma, diffuse large B cell lymphoma or T cell lymphomas. In certain embodiments, cancer is hepatocellular, ovarian, breast, lung, head and neck, non-small cell lung, brain or colorectal cancer.

Other specific cancers include, but are not limited to, advanced malignancy, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, rectal adenocarcinoma, Dukes C & D colorectal cancer, unresectable colorectal carcinoma, hepatocellular carcinoma, metastatic hepatocellular carcinoma, sarcoma, Kaposi's sarcoma, karotype acute myeloblastic leukemia, chronic lymphocytic leukemia (CLL), Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma (localized melanoma, including, but not limited to, ocular melanoma), malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, multiple myeloma, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma. In a specific embodiment, the cancer is metastatic. In another embodiment, the cancer is refractory or resistance to chemotherapy or radiation.

In one embodiment, provided herein are methods of treating, preventing or managing myelodysplastic/myeloproliferative neoplasms.

In one embodiment, provided herein are methods of treating, preventing or managing various forms of leukemias such as chronic lymphocytic leukemia, chronic myelocytic leukemia, acute lymphoblastic leukemia, acute myelogenous leukemia, hairy cell leukemia, and acute myeloblastic leukemia, including leukemias that are relapsed, refractory or resistant, which is incorporated in its entirety by reference.

The term “leukemia” refers malignant neoplasms of the blood-forming tissues. The leukemia includes, but is not limited to, chronic lymphocytic leukemia, chronic myelocytic leukemia, acute lymphoblastic leukemia, acute myelogenous leukemia and acute myeloblastic leukemia. The leukemia can be relapsed, refractory or resistant to conventional therapy. The term “relapsed” refers to a situation where patients who have had a remission of leukemia after therapy have a return of leukemia cells in the marrow and a decrease in normal blood cells. The term “refractory or resistant” refers to a circumstance where patients, even after intensive treatment, have residual leukemia cells in their marrow.

In another embodiment, provided herein are methods of treating, preventing or managing various types of lymphomas, including Non-Hodgkin's lymphoma (NHL). The term “lymphoma” refers a heterogenous group of neoplasms arising in the reticuloendothelial and lymphatic systems. “NHL” refers to malignant monoclonal proliferation of lymphoid cells in sites of the immune system, including lymph nodes, bone marrow, spleen, liver and gastrointestinal tract. In one embodiment, provided herein are methods of treating, preventing or managing aggressive and indolent subtypes of Non-Hodgkin's lymphoma (NHL). Examples of NHL include, but are not limited to, mantle cell lymphoma (MCL), lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), cutaneous t cell lymphomas, follicular lymphoma, Waldestrom macrogllobineima, plasmacytoma and any type of the mantle cell lymphomas that can be seen under the microscope (nodular, diffuse, blastic and mentle zone lymphoma).

In some embodiments, the methods provided herein are based, in part, on the discovery that the expression of certain genes or proteins associated with certain cancer cells may be utilized as biomarkers to indicate the effectiveness or progress of a disease treatment. Such cancers include, but are not limited to, lymphoma, non-Hodgkin's lymphoma, multiple myeloma, leukemia, acute myeloid leukemia (AML), and solid tumors. In certain embodiments, the cancer is of the activated B-cell phenotype in non-Hodgkin's lymphoma. In particular, these biomarkers can be used to predict, assess and track the effectiveness of patient treatment with 3-(5-amino-2-methyl-4-oxo-4H-quinazolin-3-yl)-piperidine-2,6-dione.

In some embodiments, the methods provided herein are based, in part, on the discovery that cereblon (CRBN) is associated with the anti-proliferative activities of certain drugs, such as 3-(5-amino-2-methyl-4-oxo-4H-quinazolin-3-yl)-piperidine-2,6-dione, including salts and cocrystals thereof. In some embodiments, CRBN may be utilized as a biomarker to indicate the effectiveness or progress of a disease treatment with 3-(5-amino-2-methyl-4-oxo-4H-quinazolin-3-yl)-piperidine-2,6-dione, including salts and cocrystals thereof. Without being bound by a particular theory, CRBN binding may contribute to or even be required for anti-proliferative or other activities of certain compounds, such as 3-(5-amino-2-methyl-4-oxo-4H-quinazolin-3-yl)-piperidine-2,6-dione, including salts and cocrystals thereof.

Without being limited to a particular theory, 3-(5-amino-2-methyl-4-oxo-4H-quinazolin-3-yl)-piperidine-2,6-dione, including salts and cocrystals thereof, can mediate growth inhibition, apoptosis and inhibition of angiogenic factors in certain types of cancer such as lymphoma, non-Hodkin's lymphoma, multiple myeloma, leukemia, AML, and solid tumors. Upon examining the expression of several cancer-related genes in several cell types before and after the treatment with 3-(5-amino-2-methyl-4-oxo-4H-quinazolin-3-yl)-piperidine-2,6-dione including salts and cocrystals thereof, it was discovered that the expression levels of several cancer-related genes or proteins can be used as biomarkers for predicting and monitoring cancer treatments.

It was also discovered that the level of NF-κB activity is elevated in cells of the activated B-cell phenotype in non-Hodkin's lymphoma relative to other types of lymphoma cells, and that such cells may be sensitive to treatment with 3-(5-amino-2-methyl-4-oxo-4H-quinazolin-3-yl)-piperidine-2,6-dione including salts and cocrystals thereof. This suggests that the baseline activity of NF-κB activity in lymphoma cells may be a predictive biomarker for 3-(5-amino-2-methyl-4-oxo-4H-quinazolin-3-yl)-piperidine-2,6-dione including salts and cocrystals thereof, treatment in non-Hodgkin's lymphoma patients.

Therefore, in certain embodiments, provided herein are methods for predicting tumor response to treatment in a non-Hodgkin's lymphoma patient. In one embodiment, provided herein is a method of predicting tumor response to treatment in a non-Hodgkin's lymphoma patient, the method comprising obtaining tumor tissue from the patient, purifying protein or RNA from the tumor, and measuring the presence or absence of a biomarker by ,e.g., protein or gene expression analysis. The expression monitored may be, for example, mRNA expression or protein expression. In certain embodiments, the biomarker is a gene associated with an activated B-cell phenotype of DLBCL. The genes are selected from the group consisting of IRF4/MUM1, FOXP1, SPIB, CARD11 and BLIMP/PDRM1. In one embodiment, the biomarker is NF-κB.

In another embodiment, the method comprises obtaining tumor cells from the patient, culturing the cells in the presence or absence of 3-(5-amino-2-methyl-4-oxo-4H-quinazolin-3-yl)-piperidine-2,6-dione including salts and cocrystals thereof, purifying RNA or protein from the cultured cells, and measuring the presence or absence of a biomarker by, e.g., gene or protein expression analysis.

In certain embodiments, the presence or absence of a biomarker is measured by quantitative real-time PCR (QRT-PCR), microarray, flow cytometry or immunofluorescence. In other embodiments, the presence or absence of a biomarker is measured by ELISA-based methodologies or other similar methods known in the art.

The methods provided herein encompass methods for screening or identifying cancer patients, e.g., non-Hodgkin's lymphoma patients, for treatment with 3-(5-amino-2-methyl-4-oxo-4H-quinazolin-3-yl)-piperidine-2,6-dione including salts and cocrystals thereof. In particular, provided herein are methods for selecting patients having, or who are likely to have, a higher response rate to a therapy with 3-(5-amino-2-methyl-4-oxo-4H-quinazolin-3-yl)-piperidine-2,6-dione including salts and cocrystals thereof.

In one embodiment, the method comprises the identification of patients likely to respond to therapy by obtaining tumor cells from the patient, culturing the cells in the presence or absence of 3-(5-amino-2-methyl-4-oxo-4H-quinazolin-3-yl)-piperidine-2,6-dione, including salts and cocrystals thereof, purifying RNA or protein from the cultured cells, and measuring the presence or absence of a specific biomarker. The expression monitored can be, for example, mRNA expression or protein expression. The expression in the treated sample can increase, or in some cases, decrease, for example, by about 1.5X, 2.0X, 3X, 5X, or more. In certain embodiments, the biomarker is a gene associated with an activated B-cell phenotype. The genes are selected from the group consisting of IRF4/MUM1, FOXP1, SPIB, CARD11 and BLIMP/PDRM1. In one embodiment, the biomarker is NF-κB. Baseline levels of expression of these genes can be predictive of the sensitivity of a cancer to treatment with 3-(5-amino-2-methyl-4-oxo-4H-quinazolin-3-yl)-piperidine-2,6-dione, including salts and cocrystals thereof.

In one embodiment, IRF4/MUM1 expression in cancer cells, e.g., ABC-subtype lymphoma, can be decreased with the treatment of 3-(5-amino-2-methyl-4-oxo-4H-quinazolin-3-yl)-piperidine-2,6-dione, including salts and cocrystals thereof. In some embodiments, IRF4 downregulation by 3-(5-amino-2-methyl-4-oxo-4H-quinazolin-3-yl)-piperidine-2,6-dione, including salts and cocrystals thereof, can be a potential pharmacodynamic biomarker.

In another embodiment, provided herein is a method of monitoring tumor response to treatment with 3-(5-amino-2-methyl-4-oxo-4H-quinazolin-3-yl)-piperidine-2,6-dione, including salts and cocrystals thereof, in a lymphoma, non-Hodgkin's lymphoma, multiple myeloma, leukemia, AML or a solid tumor patient. The method comprises obtaining a biological sample from the patient, measuring the expression of one or more biomarkers in the biological sample, administering 3-(5-amino-2-methyl-4-oxo-4H-quinazolin-3-yl)-piperidine-2,6-dione, including salts and cocrystals thereof, to the patient, thereafter obtaining a second biological sample from the patient, measuring biomarker expression in the second biological sample, and comparing the levels of biomarker expression, where an increased level of biomarker expression after treatment indicates the likelihood of an effective tumor response. In one embodiment, a decreased level of biomarker expression after treatment indicates the likelihood of effective tumor response. In certain embodiments, the biomarker is a gene associated with an activated B-cell phenotype of non-Hodgkin's lymphoma. The genes are selected from the group consisting of IRF4/MUM1, FOXP1, SPIB, CARD11 and BLIMP/PDRM1. In one embodiment, the biomarker is NF-κB.

In certain embodiments, the method comprises measuring the expression of one or more biomarkers genes associated with an activated B-cell phenotype. The genes are selected from the group consisting of IRF4/MUM1, FOXP1, SPIB, CARD11 and BLIMP/PDRM1. The expression monitored can be, for example, mRNA expression or protein expression. The expression in the treated sample can increase, for example, by about 1.5×, 2.0×, 3×, 5×, or more.

In yet another embodiment, a method for monitoring patient compliance with a drug treatment protocol is provided. The method comprises obtaining a biological sample from the patient, measuring the expression level of at least one biomarker in the sample, and determining if the expression level is increased or decreased in the patient sample compared to the expression level in a control untreated sample, wherein an increased or decreased expression indicates patient compliance with the drug treatment protocol. In one embodiment, the expression of one or more biomarker is increased. The expression monitored can be, for example, mRNA expression or protein expression. The expression in the treated sample can increase, for example, by about 1.5×, 2.0×, 3×, 5×, or more. In certain embodiments, the biomarker is a gene associated with an activated B-cell phenotype. The genes are selected from the group consisting of IRF4/MUM1, FOXP1, SPIB, CARD11 and BLIMP/PDRM1. In one embodiment, the biomarker is NF-κB.

In another embodiment, a method of predicting the sensitivity to treatment with 3-(5-amino-2-methyl-4-oxo-4H-quinazolin-3-yl)-piperidine-2,6-dione, including salts and cocrystals thereof, in a lymphoma, non-Hodgkin's lymphoma, multiple myeloma, leukemia, AML or a solid tumor patient is provided. In one embodiment, the patient is a non-Hodgkin's lymphoma patient, specifically, a DLBCL patient. The method comprises obtaining a biological sample from the patient, optionally isolating or purifying mRNA from the biological sample, amplifying the mRNA transcripts by, e.g., RT-PCR, where a higher baseline level of one or more specific biomarkers indicates a higher likelihood that the cancer will be sensitive to treatment with 3-(5-amino-2-methyl-4-oxo-4H-quinazolin-3-yl)-piperidine-2,6-dione. In one embodiment, the biomarker is a gene associated with an activated B-cell phenotype selected from the group consisting of IRF4/MUM1, FOXP1, SPIB, CARD11 and BLIMP/PDRM1.

In another embodiment, the method of predicting sensitivity to treatment with 3-(5-amino-2-methyl-4-oxo-4H-quinazolin-3-yl)-piperidine-2,6-dione, including salts and cocrystals thereof, in an NHL, e.g., a DLBCL patient, comprises obtaining a tumor sample from the patient, embedding the tumor sample into a paraffin-embedded, formalin-fixed block, and staining the sample with antibodies to CD20, CD10, bcl-6, IRF4/MUM1, bcl-2, cyclin D2, and/or FOXP1, as described in Hans et al., Blood, 2004, 103: 275-282, which is hereby incorporated by reference in its entirety. In one embodiment, CD10, bcl-6, and IRF4/MUM-1 staining can be used to divide DLBCL into GCB and non-GCB subgroups to predict an outcome.

In one embodiment, provided herein is a method for predicting tumor response to treatment in a non-Hodgkin's lymphoma patient, comprising:

(i) obtaining a biological sample from the patient;

(ii) measuring activity of the NF-κB pathway in the biological sample; and

(iii) comparing the level of NF-κB activity in the biological sample to that of a biological sample of a non-activated B-cell lymphoma subtype; wherein an increased level of NF-κB activity relative to non-activated B-cell subtype lymphoma cells indicates a likelihood of an effective patient tumor response to treatment with 3-(5-amino-2-methyl-4-oxo-4H-quinazolin-3-yl)-piperidine-2,6-dione, including salts and cocrystals thereof.

In one embodiment, measuring activity of the NF-κB pathway in the biological sample comprises measuring the level of NF-κB in the biological sample.

In one embodiment, provided herein is a method of monitoring tumor response to treatment in a non-Hodgkin's lymphoma patient, comprising:

(i) obtaining a biological sample from the patient;

(ii) measuring the level of NF-κB activity in the biological sample;

(iii) administering a therapeutically effective amount of 3-(5-amino-2-methyl-4-oxo-4H-quinazolin-3-yl)-piperidine-2,6-dione, including salts and cocrystals thereof to the patient;

(iv) obtaining a second biological sample from the patient;

(v) measuring the level of NF-κB activity in the second biological sample; and

(vi) comparing the level of NF-κB activity in the first biological sample to that in the second biological sample;

wherein a decreased level of NF-κB activity in the second biological sample relative to the first biological sample indicates a likelihood of an effective patient tumor response.

In one embodiment, provided herein is a method for monitoring patient compliance with a drug treatment protocol in a non-Hodgkin's lymphoma patient, comprising:

(i) obtaining a biological sample from the patient;

(ii) measuring the level of NF-κB activity in the biological sample; and

(iii) comparing the level of NF-κB activity in the biological sample to a control untreated sample;

wherein a decreased level of NF-κB activity in the biological sample relative to the control indicates patient compliance with the drug treatment protocol.

In one embodiment, the non-Hodgkin's lymphoma is diffuse large B-cell lymphoma.

In another embodiment, the level of NF-κB activity is measured by an enzyme-linked immunosorbent assay.

In one embodiment, provided herein is a method for predicting tumor response to treatment in a non-Hodgkin's lymphoma patient, comprising:

(i) obtaining a biological sample from the patient;

(ii) culturing cells from the biological sample;

(iii) purifying RNA from the cultured cells; and

(iv) identifying increased expression of a gene associated with the activated B-cell phenotype of non-Hodgkin's lymphoma relative to control non-activated B-cell phenotype of non-Hodgkin's lymphoma;

wherein increased expression of a gene associated with the activated B-cell phenotype of non-Hodgkin's lymphoma indicates a likelihood of an effective patient tumor response to treatment with 3-(5-amino-2-methyl-4-oxo-4H-quinazolin-3-yl)-piperidine-2,6-dione, including salts and cocrystals thereof.

In one embodiment, increased expression is an increase of about 1.5×, 2.0×, 3×, 5×, or more.

In one embodiment, the gene associated with the activated B-cell phenotype is selected from the group consisting of IRF4/MUM1, FOXP1, SPIB, CARD11 and BLIMP/PDRM1.

In one embodiment, identifying the expression of a gene associated with the activated B-cell phenotype of non-Hodgkin's lymphoma is performed by quantitative real-time PCR.

Also provided herein is a method for treating or managing non-Hodgkin's lymphoma, comprising:

(i) identifying a patient having non-Hodgkin's lymphoma sensitive to treatment with 3-(5-amino-2-methyl-4-oxo-4H-quinazolin-3-yl)-piperidine-2,6-dione, including salts and cocrystals thereof and

(ii) administering to the patient a therapeutically effective amount of 3-(5-amino-2-methyl-4-oxo-4H-quinazolin-3-yl)-piperidine-2,6-dione, including salts and cocrystals thereof.

In one embodiment, the non-Hodgkin's lymphoma is diffuse large B-cell lymphoma.

In another embodiment, the non-Hodgkin's lymphoma is of the activated B-cell (ABC) phenotype.

In another embodiment, the non-Hodgkin's lymphoma is of the germinal center B-cell (GCB) phenotype.

In another embodiment, the diffuse large B-cell lymphoma is characterized by the expression of one or more biomarkers overexpressed in RIVA, U2932, TMD8, OCI-Ly3 or OCI-Ly10 cell lines.

In another embodiment, the diffuse large B-cell lymphoma is characterized by the expression of one or more biomarkers overexpressed in RIVA, U2932, TMD8 or OCI-Ly10 cell lines.

In one embodiment, identifying a patient having lymphoma sensitive to treatment with 3-(5-amino-2-methyl-4-oxo-4H-quinazolin-3-yl)-piperidine-2,6-dione, including salts and cocrystals thereof, comprises characterization of the lymphoma phenotype of the patient.

In one embodiment, the lymphoma phenotype is characterized as an activated B-cell subtype.

In one embodiment, the lymphoma phenotype is characterized as an activated B-cell subtype of diffuse large B-cell lymphoma.

In certain embodiments, identification of the lymphoma phenotype comprises obtaining a biological sample from a patient having lymphoma. In one embodiment, the biological sample is a cell culture or tissue sample. In one embodiment, the biological sample is a sample of tumor cells. In another embodiment, the biological sample is a lymph node biopsy, a bone marrow biopsy, or a sample of peripheral blood tumor cells. In one embodiment, the biological sample is a blood sample.

In one embodiment, identifying a patient having non-Hodgkin's lymphoma sensitive to treatment with 3-(5-amino-2-methyl-4-oxo-4H-quinazolin-3-yl)-piperidine-2,6-dione, including salts and cocrystals thereof, comprises identification of a gene associated with an activated B-cell phenotype. In one embodiment, the gene associated with the activated B-cell phenotype is selected from the group consisting of IRF4/MUM1, FOXP1, SPIB, CARD11 and BLIMP/PDRM1.

In one embodiment, identifying a patient having non-Hodgkin's lymphoma sensitive to treatment with 3-(5-amino-2-methyl-4-oxo-4H-quinazolin-3-yl)-piperidine-2,6-dione, including salts and cocrystals thereof, comprises measuring the level of NF-κB activity in the patient. In another embodiment, measuring the level of NF-κB activity in a patient comprises measuring the baseline NF-κB activity level in tumor cells obtained from the patient.

In another embodiment, the diffuse large B-cell lymphoma is characterized by one or more of the following:

(i) over expression of SPIB, a hematopoietic-specific Ets family transcription factor required for survival of activated B-cell subtype cells;

(ii) higher constitutive IRF4/MUM1 expression than GCB subtype cells;

(iii) higher constitutive FOXP1 expression up-regulated by trisomy 3;

(iv) higher constitutive Blimpl, i.e., PRDM1, expression; and

(v) higher constitutive CARD11 gene expression; and

(vi) an increased level of NF-κB activity relative to non-activated B-cell subtype DLBCL cells.

Additional prognostic factors that may be used concurrently with those provided herein are prognostic factors of disease (tumor) burden, absolute lymphocyte count (ALC), time since last rituximab therapy for lymphomas, or all of the above.

Also provided herein is a method of selecting a group of cancer patients based on the level of CRBN expression, or the levels of DDB1, DDB2, IRF4 or NFκB expression within the cancer, for the purposes of predicting clinical response, monitoring clinical response, or monitoring patient compliance to dosing by 3-(5-amino-2-methyl-4-oxo-4H-quinazolin-3-yl)-piperidine-2,6-dione, including salts and cocrystals thereof; wherein the cancer patients are selected from multiple myeloma, non-Hodgkin's lymphoma, diffuse large B-cell lymphoma, melanoma and solid tumor patients. Baseline levels of expression of these genes can be predictive of the sensitivity of a cancer to treatment with 3-(5-amino-2-methyl-4-oxo-4H-quinazolin-3-yl)-piperidine-2,6-dione, including salts and cocrystals thereof.

In one embodiment, IRF4/MUM1 expression in cancer cells, e.g., ABC-subtype lymphoma, can be decreased with the treatment of 3-(5-amino-2-methyl-4-oxo-4H-quinazolin-3-yl)-piperidine-2,6-dione, including salts and cocrystals thereof. In some embodiments, IRF4 downregulation by 3-(5-amino-2-methyl-4-oxo-4H-quinazolin-3-yl)-piperidine-2,6-dione, including salts and cocrystals thereof, can be a potential pharmacodynamic biomarker.

In one embodiment, the cancer patients are multiple myeloma patients.

In one embodiment, the cancer patients are non-Hodgkin's lymphoma patients.

In another embodiment, the method comprises selecting a group of cancer patients responsive to treatment with 3-(5-amino-2-methyl-4-oxo-4H-quinazolin-3-yl)-piperidine-2,6-dione, including salts and cocrystals thereof; based on the level of CRBN expression, or the levels of DDB1, DDB2, IRF4 or NFκB expression within the patient's T cells, B cells, or plasma cells, for the purposes of predicting clinical response, monitoring clinical response, or monitoring patient compliance to dosing by 3-(5-amino-2-methyl-4-oxo-4H-quinazolin-3-yl)-piperidine-2,6-dione, including salts and cocrystals thereof.

In one embodiment, the cancer patients are selected from multiple myeloma, non-Hodgkin's lymphoma, diffuse large B-cell lymphoma, melanoma, hepatocellular cancer, and solid tumor patients.

Also provided herein are methods of treating cancer, e.g., lymphoma, non-Hodgkin's lymphoma, multiple myeloma, leukemia, acute myeloid leukemia (AML), and solid tumors, which result in an improvement in overall survival of the patient. In some embodiments, the improvement in overall survival of the patient is observed in a patient population sensitive to treatment with 3-(5-amino-2-methyl-4-oxo-4H-quinazolin-3-yl)-piperidine-2,6-dione, including salts and cocrystals thereof. In some embodiments, the patient population sensitive to treatment with 3-(5-amino-2-methyl-4-oxo-4H-quinazolin-3-yl)-piperidine-2,6-dione, including salts and cocrystals thereof, is characterized by one or more biomarkers provided herein.

In other embodiments, provided herein are methods of treating cancer, e.g., lymphoma, non-Hodgkin's lymphoma, multiple myeloma, diffuse large B-cell lymphoma, follicular lymphoma, mantle cell lymphoma, leukemia, acute myeloid leukemia (AML), and breast or hepatpocellular tumor tumors, which result in disease free survival of the patient. In some embodiments, disease free survival of the patient is observed in a patient population sensitive to treatment with 3-(5-amino-2-methyl-4-oxo-4H-quinazolin-3-yl)-piperidine-2,6-dione, including salts and cocrystals thereof. In some embodiments, the patient population sensitive to treatment with 3-(5-amino-2-methyl-4-oxo-4H-quinazolin-3-yl)-piperidine-2,6-dione, including salts and cocrystals thereof, is characterized by one or more biomarkers provided herein.

In other embodiments, provided herein are methods of treating cancer, e.g., lymphoma, non-Hodgkin's lymphoma, multiple myeloma, leukemia, acute myeloid leukemia (AML), and solid tumors such as hepatocellular cancer, which result in an improvement in the objective response rate in the patient population. In some embodiments, the patient population sensitive to treatment with 3-(5-amino-2-methyl-4-oxo-4H-quinazolin-3-yl)-piperidine-2,6-dione, including salts and cocrystals thereof. In some embodiments, the patient population sensitive to treatment with 3-(5-amino-2-methyl-4-oxo-4H-quinazolin-3-yl)-piperidine-2,6-dione, including salts and cocrystals thereof, is characterized by one or more biomarkers provided herein.

In other embodiments, provided herein are methods of treating cancer, e.g., lymphoma, non-lymphoma, Hodgkin's lymphoma, multiple myeloma, leukemia, acute myeloid leukemia (AML), and solid tumors including hepatocellular cancer, which result in an improved time to progression or progression-free survival of the patient. In some embodiments, the improved time to progression or progression-free survival of the patient is observed in a patient population sensitive to treatment with 3-(5-amino-2-methyl-4-oxo-4H-quinazolin-3-yl)-piperidine-2,6-dione, including salts and cocrystals thereof. In some embodiments, the patient population sensitive to treatment with 3-(5-amino-2-methyl-4-oxo-4H-quinazolin-3-yl)-piperidine-2,6-dione, including salts and cocrystals thereof, is characterized by one or more biomarkers provided herein.

In some embodiments, the methods for treating, preventing and/or managing lymphomas provided herein may be used in patients that have not responded to standard treatment. In one embodiment, the lymphoma is relapsed, refractory or resistant to conventional therapy.

In other embodiments, the methods for treating, preventing and/or managing lymphomas provided herein may be used in treatment naive patients, i.e., patients that have not yet received treatment.

In one embodiment, provided herein are methods of modulating, e.g., reducing, lymphocytic activity, including activity of B cells and/or T cells, comprising contacting B cell and/or T cell with an effective amount of a solid form comprising a salt or cocrystal of Compound 1.

Examples of TNFα related disorders include, but are not limited to: endotoxemia or toxic shock syndrome; cachexia; adult respiratory distress syndrome; bone resorption diseases such as arthritis; hypercalcemia; Graft versus Host Reaction; cerebral malaria; inflammation; tumor growth; chronic pulmonary inflammatory diseases; reperfusion injury; myocardial infarction; stroke; circulatory shock; rheumatoid arthritis; Crohn's disease; HIV infection and AIDS; other disorders such as rheumatoid arthritis, rheumatoid spondylitis, osteoarthritis, psoriatic arthritis and other arthritic conditions, septic shock, septis, endotoxic shock, graft versus host disease, wasting, Crohn's disease, ulcerative colitis, multiple sclerosis, systemic lupus erythromatosis, ENL in leprosy, HIV, AIDS, and opportunistic infections in AIDS; disorders such as septic shock, sepsis, endotoxic shock, hemodynamic shock and sepsis syndrome, post ischemic reperfusion injury, malaria, mycobacterial infection, meningitis, psoriasis, congestive heart failure, fibrotic disease, cachexia, graft rejection, oncogenic or cancerous conditions, asthma, autoimmune disease, radiation damages, and hyperoxic alveolar injury; viral infections, such as those caused by the herpes viruses; viral conjunctivitis; or atopic dermatitis.

In certain embodiments, a therapeutically or prophylactically effective amount of the solid form comprising a salt or cocrystal of Compound 1 is from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 0.02 to about 25 mg per day, or from about 0.05 to about 10 mg per day

In certain embodiment, a therapeutically or prophylactically effective amount is from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 0.02 to about 25 mg per day, or from about 0.05 to about 10 mg every other day

In certain embodiments, the therapeutically or prophylactically effective amount is about 1, about 2, about 5, about 10, about 15, about 20, about 25, about 30, about 40, about 45, about 50, about 60, about 70, about 80, about 90, about 100, or about 150 mg per day.

In one embodiment, a solid form comprising a salt or cocrystal of Compound 1 provided herein is administered daily and continuously for three or four weeks at a dose of from about 0.1 mg to about 500 mg per day, followed by a rest of one or two weeks. In other embodiments, the dose can be from about 1 mg to about 300 mg, from about 0.1 mg to about 150 mg, from about 1 mg to about 200 mg, from about 10 mg to about 100 mg, from about 0.1 mg to about 50 mg, from about 1 mg to about 50 mg, from about 10 mg to about 50 mg, from about 20 mg to about 30 mg, or from about 1 mg to about 20 mg, followed by a rest.

In one embodiment, the recommended daily dose range of the solid form comprising a salt or cocrystal of Compound 1 for the conditions described herein lie within the range of from about 0.5 mg to about 50 mg per day, preferably given as a single once-a-day dose, or in divided doses throughout a day. In some embodiments, the dosage ranges from about 1 mg to about 50 mg per day. In other embodiments, the dosage ranges from about 0.5 to about 5 mg per day. Specific doses per day include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg per day.

In a specific embodiment, the recommended starting dosage may be 0.5, 1, 2, 3, 4, 5, 10, 15, 20, 25 or 50 mg per day. In another embodiment, the recommended starting dosage may be 0.5, 1, 2, 3, 4, or 5 mg per day. The dose may be escalated to 15, 20, 25, 30, 35, 40, 45 and 50 mg/day. In a specific embodiment, the compound can be administered in an amount of about 25 mg/day to patients with NHL (e.g., DLBCL). In a particular embodiment, the compound can be administered in an amount of about 10 mg/day to patients with NHL (e.g., DLBCL).

In certain embodiments, the therapeutically or prophylactically effective amount is from about 0.001 to about 100 mg/kg/day, from about 0.01 to about 50 mg/kg/day, from about 0.01 to about 25 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, or from about 0.01 to about 1 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, www.fda.gov/cder/cancer/animalframe.htm). For example, a dose of 1 mg/kg/day for a 65 kg human is approximately equal to 38 mg/m²/day

In certain embodiments, the amount of the compound administered is sufficient to provide a plasma concentration of the compound at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In other embodiments, the amount of the solid form comprising a salt or cocrystal of Compound 1 administered is sufficient to provide a plasma concentration of the solid form comprising a salt or cocrystal of Compound 1 at steady state, ranging from about 5 to about 100 nM, about 5 to about 50 nM, about 10 to about 100 nM, about 10 to about 50 nM or from about 50 to about 100 nM

As used herein, the term “plasma concentration at steady state” is the concentration reached after a period of administration of a compound provided herein, e.g., the solid form comprising a salt or cocrystal of Compound 1. Once steady state is reached, there are minor peaks and troughs on the time dependent curve of the plasma concentration of the solid form comprising a salt or cocrystal of Compound 1

In certain embodiments, the amount of the solid form comprising a salt or cocrystal of Compound 1 administered is sufficient to provide a maximum plasma concentration (peak concentration) of the solid form comprising a salt or cocrystal of Compound 1, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM,or from about 1 to about 20 μM

In certain embodiments, the amount of the solid form comprising a salt or cocrystal of Compound 1 administered is sufficient to provide a minimum plasma concentration (trough concentration) of the compound, ranging from about 0.001 to about 500 μM, about 0.002 to about 200 μM, about 0.005 to about 100 μM, about 0.01 to about 50 μM, from about 1 to about 50 μM, about 0.01 to about 25 μM, from about 0.01 to about 20 μM, from about 0.02 to about 20 μM, from about 0.02 to about 20 μM, or from about 0.01 to about 20 μM

In certain embodiments, the amount of the solid form comprising a salt or cocrystal of Compound 1 administered is sufficient to provide an area under the curve (AUC) of the compound, ranging from about 100 to about 100,000 ng*hr/mL, from about 1,000 to about 50,000 ng*hr/mL, from about 5,000 to about 25,000 ng*hr/mL, or from about 5,000 to about 10,000 ng*hr/mL

In certain embodiments, the patient to be treated with one of the methods provided herein has not been treated with anticancer therapy prior to the administration of the solid form comprising a salt or cocrystal of Compound 1. In certain embodiments, the patient to be treated with one of the methods provided herein has been treated with anticancer therapy prior to the administration of the solid form comprising a salt or cocrystal of Compound 1. In certain embodiments, the patient to be treated with one of the methods provided herein has developed drug resistance to the anticancer therapy

The methods provided herein encompass treating a patient regardless of patient's age, although some diseases or disorders are more common in certain age groups. Further provided herein is a method for treating a patient who has undergone surgery in an attempt to treat the disease or condition at issue, as well in one who has not. Because the subjects with cancer have heterogeneous clinical manifestations and varying clinical outcomes, the treatment given to a particular subject may vary, depending on his/her prognosis. The skilled clinician will be able to readily determine without undue experimentation, specific secondary agents, types of surgery, and types of non-drug based standard therapy that can be effectively used to treat an individual subject with cancer.

Depending on the disease to be treated and the subject's condition, the solid form comprising a salt or cocrystal of Compound 1 may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. The solid form comprising a salt or cocrystal of Compound 1 may be formulated, alone or together, in suitable dosage unit with pharmaceutically acceptable excipients, carriers, adjuvants and vehicles, appropriate for each route of administration

In one embodiment, the solid form comprising a salt or cocrystal of Compound 1 is administered orally. In another embodiment, the solid form comprising a salt or cocrystal of Compound 1 is administered parenterally. In yet another embodiment, the solid form comprising a salt or cocrystal of Compound 1 is administered intravenously.

The solid form comprising a salt or cocrystal of Compound 1 can be delivered as a single dose such as, e.g., a single bolus injection, or oral tablets or pills; or over time, such as, e.g., continuous infusion over time or divided bolus doses over time. The solid form comprising a salt or cocrystal of Compound 1 can be administered repeatedly if necessary, for example, until the patient experiences stable disease or regression, or until the patient experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 92(3): 205-216 (2000). Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

A solid form comprising a salt or cocrystal of Compound 1 can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as a solid form comprising a salt or cocrystal of Compound 1, is administered once or more than once each day, for example, for a period of time. The term “continuous” is intended to mean that a therapeutic compound, such as a solid form comprising a salt or cocrystal of Compound 1, is administered daily for an uninterrupted period of at least 10 days to 52 weeks. The term “intermittent” or “intermittently” as used herein is intended to mean stopping and starting at either regular or irregular intervals. For example, intermittent administration of a solid form comprising a salt or cocrystal of Compound 1, is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as a solid form comprising a salt or cocrystal of Compound 1, is administered daily or continuously but with a rest period. Cycling therapy is described elsewhere herein.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, a solid form comprising a salt or cocrystal of Compound 1, is administered once a day. In another embodiment, a solid form comprising a salt or cocrystal of Compound 1, is administered twice a day. In yet another embodiment, a solid form comprising a salt or cocrystal of Compound 1, is administered three times a day. In still another embodiment, a solid form comprising a salt or cocrystal of Compound 1, is administered four times a day.

In certain embodiments, a solid form comprising a salt or cocrystal of Compound 1, is administered once per day from one day to six months, from one week to three months, from one week to four weeks, from one week to three weeks, or from one week to two weeks. In certain embodiments, a solid form comprising a salt or cocrystal of Compound 1, is administered once per day for one week, two weeks, three weeks, or four weeks. In one embodiment, a solid form comprising a salt or cocrystal of Compound 1, is administered once per day for one week. In another embodiment, a solid form comprising a salt or cocrystal of Compound 1, is administered once per day for two weeks. In yet another embodiment, a solid form comprising a salt or cocrystal of Compound 1, is administered once per day for three weeks. In still another embodiment, a solid form comprising a salt or cocrystal of Compound 1, is administered once per day for four weeks.

### 5.3. COMBINATION THERAPY WITH A SECOND ACTIVE AGENT

A solid form comprising a salt or cocrystal of Compound 1 provided herein can be combined with other pharmacologically active compounds (“second active agents”),optionally in combination with radiation therapy, blood transfusions, or surgery, in methods and compositions provided herein. Certain combinations may work synergistically in the treatment of particular types of diseases or disorders, and conditions and symptoms associated with such diseases or disorders. A solid form comprising a salt or cocrystal of Compound 1 provided herein can also work to alleviate adverse effects associated with certain second active agents, and vice versa.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a patient with a disease or disorder. A first therapy (e.g., a prophylactic or therapeutic agent such as a solid form comprising a salt or cocrystal of Compound 1 provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

Administration of a solid form comprising a salt or cocrystal of Compound 1 and one or more second active agents to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the cancer being treated.

The route of administration of a solid form comprising a salt or cocrystal of Compound 1 is independent of the route of administration of a second therapy. In one embodiment, the solid form comprising a salt or cocrystal of Compound 1 is administered orally. In another embodiment, the solid form comprising a salt or cocrystal of Compound 1 is administered intravenously. Thus, in accordance with these embodiments, the solid form comprising a salt or cocrystal of Compound 1 is administered orally or intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In one embodiment, the solid form comprising a salt or cocrystal of Compound 1 and a second therapy are administered by the same mode of administration, orally or by IV. In another embodiment, the compound of Formula I is administered by one mode of administration, e.g., by IV, whereas the second agent (an anticancer agent) is administered by another mode of administration, e.g., orally.

In one embodiment, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated or managed, the severity and stage of disease, and the amount of the compound of Formula I provided herein and any optional additional active agents concurrently administered to the patient.

One or more second active ingredients or agents can be used in the methods and compositions provided herein. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies, particularly, therapeutic antibodies to cancer antigens. Typical large molecule active agents are biological molecules, such as naturally occurring or synthetic or recombinant proteins. Proteins that are particularly useful in the methods and compositions provided herein include proteins that stimulate the survival and/or proliferation of hematopoietic precursor cells lymphopoietic cells in vitro or in vivo. Other useful proteins stimulate the division and differentiation of committed hematopoietic progenitors in cells in vitro or in vivo. Particular proteins include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; GM-CF and GM-CSF; and EPO.

In certain embodiments, GM-CSF, G-CSF, SCF or EPO is administered subcutaneously during about five days in a four or six week cycle in an amount ranging from about 1 to about 750 mg/m²/day, from about 25 to about 500 mg/m²/day, from about 50 to about 250 mg/m²/day, or from about 50 to about 200 mg/m²/day. In certain embodiments, GM-CSF may be administered in an amount of from about 60 to about 500 mcg/m² intravenously over 2 hours or from about 5 to about 12 mcg/m²/day subcutaneously. In certain embodiments, G-CSF may be administered subcutaneously in an amount of about 1 mcg/kg/day initially and can be adjusted depending on rise of total granulocyte counts. The maintenance dose of G-CSF may be administered in an amount of about 300 (in smaller patients) or 480 mcg subcutaneously. In certain embodiments, EPO may be administered subcutaneously in an amount of 10,000 Unit 3 times per week.

Particular proteins that can be used in the methods and compositions include, but are not limited to: filgrastim, which is sold in the United States under the trade name Neupogen® (Amgen, Thousand Oaks, Calif.); sargramostim, which is sold in the United States under the trade name Leukine® (Immunex, Seattle, Wash.); and recombinant EPO, which is sold in the United States under the trade name Epogen® (Amgen, Thousand Oaks, Calif.) or Procrit® (Johnson and Johnson, New Brunswick, N.J.).

Recombinant and mutated forms of GM-CSF can be prepared as described in U.S. Pat. Nos. 5,391,485; 5,393,870; and 5,229,496; all of which are incorporated herein by reference. Recombinant and mutated forms of G-CSF can be prepared as described in U.S. Pate. Nos. 4,810,643; 4,999,291; 5,528,823; and 5,580,755; the entireties of which are incorporated herein by reference.

Also provided for use in combination with a solid form comprising a salt or cocrystal of Compound 1 provided herein are native, naturally occurring, and recombinant proteins. Further encompassed are mutants and derivatives (e.g., modified forms) of naturally occurring proteins that exhibit, in vivo, at least some of the pharmacological activity of the proteins upon which they are based. Examples of mutants include, but are not limited to, proteins that have one or more amino acid residues that differ from the corresponding residues in the naturally occurring forms of the proteins. Also encompassed by the term “mutants” are proteins that lack carbohydrate moieties normally present in their naturally occurring forms (e.g., nonglycosylated forms). Examples of derivatives include, but are not limited to, pegylated derivatives and fusion proteins, such as proteins formed by fusing IgG1 or IgG3 to the protein or active portion of the protein of interest. See, e.g., Penichet, M. L. and Morrison, S. L., J. Immunol. Methods 248:91-101 (2001).

Antibodies that can be used in combination with a solid form comprising a salt or cocrystal of Compound 1 provided herein include monoclonal and polyclonal antibodies. Examples of antibodies include, but are not limited to, trastuzumab (Herceptin®), rituximab (Rituxan™), bevacizumab (Avastin™), pertuzumab (Omnitarg™), tositumomab (Bexxar™), edrecolomab (Panorex®), and G250. A solid form comprising a salt or cocrystal of Compound lcan also be combined with, or used in combination with, anti-TNF-α antibodies, and/or anti-EGFR antibodies, such as, for example, ^{Erbitux}® (cetuximab) or panitumumab.

Antibodies that can be used in combination with a solid form comprising a salt or cocrystal of Compound 1 provided herein include immune checkpoint inhibitors, such as, anti-CTLA4, anti-PD1, anti-PD-L1, anti-Tim-3, anti-Lag-3 antibodies. In some such embodiments, the PD-1 or PD-L1 antibodies are, for example, avelumab, durvalumab, MEDI0680, atezolizumab, BMS-936559, nivolumab, pembrolizumab, pidilizumab, or PDR-001. In one such embodiment, the anti-Lag-3 antibody is BMS-986016.

Additional antibodies that can be used in combination with a solid form comprising a salt or cocrystal of Compound 1 provided herein include anti-RSPO antibodies.

Large molecule active agents may be administered in the form of anti-cancer vaccines. For example, vaccines that secrete, or cause the secretion of, cytokines such as IL-2, G-CSF, and GM-CSF can be used in the methods and pharmaceutical compositions provided. See, e.g., Emens, L.A., et al., Curr. Opinion Mol. Ther. 3(1):77-84 (2001).

Second active agents that are small molecules can also be used to alleviate adverse effects associated with the administration of a solid form comprising a salt or cocrystal of Compound 1 provided herein. However, like some large molecules, many are believed to be capable of providing an additive or synergistic effect when administered with (e.g., before, after or simultaneously) a solid form comprising a salt or cocrystal of Compound 1 provided herein. Examples of small molecule second active agents include, but are not limited to, anti-cancer agents, antibiotics, immunosuppressive agents, and steroids.

In certain embodiments, the second agent is a BRAF inhibitor, an HSP inhibitor, a proteasome inhibitor, a FLT3 inhibitor, a MEK inhibitor, a PI3K inhibitor, an EGFR inhibitor, an immunomodulatory compound, or a TOR kinase inhibitor. In some such embodiments, the BRAF inhibitor is sorafenib, dabrafenib, encorafenib, or vemurafenib. In some such embodiment, the HSP inhibitor is geldanamycin, gamitrinib, luminespib, or radicicol. In some embodiments, the proteasome inhibitor is bortezomib, carfilzomib, ixazomib, disulfiram, oprozomib, delanzomib, or ixazomib. In other embodiments, the FLT3 inhibitor is quizartinib, midostaurin, sorafenib , sunitinib, or lestaurtinib. In some such embodiments, the MEK inhibitor is trametinib, cobimetinib, binimetinib, selumetinib, PD-325901, CI-1040 (PD184352) or TAK-733. In some other embodiments, the PI3K inhibitor is AT7867, AZD 8055, BX-912, silmitasertib, pictilisib, MK-2206, or pilaralisib. In another embodiment, the EGFR inhibitor is gefitinib, erlotinib, afatinib, osimertinib (Tagrisso™), rociletinib, or lapatinib. In some other embodiments, the TOR kinase inhibitor is CC115 (1-ethyl-7-(2-methyl-6-(1H-1,2,4-triazol-3-yl)pyridin-3-yl)-3,4-dihydropyrazino[2,3-b]pyrazin-2(1H)-one), CC223 (7-(6-(2-hydroxypropan-2-yl)pyridin-3-yl)-1-((trans)-4-methoxycyclohexyl)-3,4-dihydropyrazino[2,3-b]pyrazin-2(1H)-one), OSI-027, AZD8055, sapanisertib, dactolisib, BGT226, voxtalisib (SAR-245409), apitolisib, omipalisib (GSK-2126458), PF-04691502, gedatolisib or PP242. In some embodiments, the immunomodulatory compound is thalidomide, lenalidomide, pomalidomide, CC220, or CC122.

Examples of additional anti-cancer agents to be used within the methods or compositions described herein include, but are not limited to: acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; clofarabine; crisnatol mesylate; cyclophosphamide; cytarabine; dacarbazine; dabrafenib; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; omacetaxine; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sorafenib; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; vemurafenib; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs to be included within the methods or compositions include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogens, prostatic carcinoma; antiestrogens; antineoplastons; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitors; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogs; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; Ara-C ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogs; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., Gleevec®); imiquimod; immunostimulant peptides; insulin-like growth factor-1 receptor inhibitors; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogs; lipophilic disaccharide peptides; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitors; mifepristone; miltefosine; mirimostim; mitoguazone; mitolactol; mitomycin analogs; mitonafide; mitotoxin fibroblast growth factor-saporin; mitoxantrone; mofarotene; molgramostim; cetuximab, human chorionic gonadotrophin; monophosphoryl lipid A and mycobacterium cell wall skeleton; mopidamol; mustard anticancer agents; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidants; nitrullyn; oblimersen (Genasense®); O⁶-benzylguanine; octreotide; okicenone; oligonucleotides; onapristone; ondansetron; ondansetron; oracin; oral cytokine inducers; ormaplatin; osaterone; oxaliplatin; oxaunomycin; paclitaxel; paclitaxel analogs; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitors; platinum complexes; platinum compounds; platinum-triamine complexes; porfimer sodium; porfiromycin; prednisone; propyl bis-acridone; prostaglandin J2; proteasome inhibitors; protein A-based immune modulators; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugates; raf antagonists; raltitrexed; ramosetron; ras farnesyl protein transferase inhibitors; ras inhibitors; ras-GAP inhibitors; retelliptine demethylated; rhenium Re 186 etidronate; rhizoxin; ribozymes; RH retinamide; rohitukine; romurtide; roquinimex; rubiginone B 1; ruboxyl; safingol; saintopin; sarmustine; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonists; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonists; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

Specific second active agents particularly useful in the methods or compositions include, but are not limited to, rituximab, oblimersen (Genasense), remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, Iressa®, taxol, taxotere, fluorouracil, leucovorin, irinotecan, xeloda, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, interleukin 2, ganulocyte-macrophage colony-stimulating factor, dacarbazine, vinorelbine, zoledronic acid, palmitronate, biaxin, busulphan, prednisone, bisphosphonate, arsenic trioxide, vincristine, doxorubicin (Doxil®), paclitaxel, ganciclovir, adriamycin, estramustine sodium phosphate (Emcyt®), sulindac, and etoposide.

Other specific second active agents particularly useful in the methods or compositions include, but are not limited to, sorafenib, dabrafenib, vemurafenib, trametinib, cobimetinib, binimetinib, selumetinib, PD-325901, CI-1040 (PD184352), TAK-733, AT7867, AZD 8055, BX-912, silmitasertib, pictilisib, MK-2206, pilaralisib, gefitinib, erlotinib, lapatinib, osimertinib, CC115, CC223, OSI-027, AZD8055, sapanisertib, Dactolisib, BGT226, voxtalisib, apitolisib, omipalisib, PF-04691502, gedatolisib, PP242, lenalidomide, pomalidomide, or CC122.

Other specific second active agents particularly useful in the methods or compositions include, but are not limited to, avelumab, durvalumab, MEDI0680, atezolizumab, BMS-936559, nivolumab, pembrolizumab, pidilizumab, PDR-001, sorafenib, cetuximab, panatumumab, erlotinib, trametinib, trastuzumab, CC223, CC122 or lapatinib.

In certain embodiments of the methods provided herein, use of a second active agent in combination with a solid form comprising a salt or cocrystal of Compound 1 provided herein may be modified or delayed during or shortly following administration of a solid form comprising a salt or cocrystal of Compound 1 provided herein as deemed appropriate by the practitioner of skill in the art. In certain embodiments, subjects being administered a solid form comprising a salt or cocrystal of Compound 1 provided herein alone or in combination with other therapies may receive supportive care including antiemetics, myeloid growth factors, and transfusions of blood products, when appropriate. In some embodiments, subjects being administered an a solid form comprising a salt or cocrystal of Compound 1 provided herein may be administered a growth factor as a second active agent according to the judgment of the practitioner of skill in the art.

In certain embodiments, a solid form comprising a salt or cocrystal of Compound 1 provided herein is administered with gemcitabine, cisplatinum, 5-fluorouracil, mitomycin, methotrexate, vinblastine, doxorubicin, carboplatin, thiotepa, paclitaxel or docetaxel to patients with locally advanced or metastatic urothelial carcinoma.

In certain embodiments, a solid form comprising a salt or cocrystal of Compound 1 is administered in combination with a second active ingredient as follows: temozolomide to pediatric patients with relapsed or progressive brain tumors or recurrent neuroblastoma; celecoxib, etoposide and cyclophosphamide for relapsed or progressive CNS cancer; temozolomide to patients with recurrent or progressive meningioma, malignant meningioma, hemangiopericytoma, multiple brain metastases, relapsed brain tumors, or newly diagnosed glioblastoma multiforme; irinotecan to patients with recurrent glioblastoma; carboplatin to pediatric patients with brain stem gliomas; procarbazine to pediatric patients with progressive malignant gliomas; cyclophosphamide to patients with poor prognosis malignant brain tumors, newly diagnosed or recurrent glioblastoma multiforms; carmustine (Gliadel®) for high grade recurrent malignant gliomas; temozolomide and tamoxifen for anaplastic astrocytoma; or topotecan for gliomas, glioblastoma, anaplastic astrocytoma or anaplastic oligodendrogliomatemozolomide to pediatric patients with relapsed or progressive brain tumors or recurrent neuroblastoma; celecoxib, etoposide and cyclophosphamide for relapsed or progressive CNS cancer; temozolomide to patients with recurrent or progressive meningioma, malignant meningioma, hemangiopericytoma, multiple brain metastases, relapsed brain tumors, or newly diagnosed glioblastoma multiforme; irinotecan to patients with recurrent glioblastoma; carboplatin to pediatric patients with brain stem gliomas; procarbazine to pediatric patients with progressive malignant gliomas; cyclophosphamide to patients with poor prognosis malignant brain tumors, newly diagnosed or recurrent glioblastoma multiforms; carmustine (Gliadel®) for high grade recurrent malignant gliomas; temozolomide and tamoxifen for anaplastic astrocytoma; or topotecan for gliomas, glioblastoma, anaplastic astrocytoma or anaplastic oligodendroglioma.

In certain embodiments, a solid form comprising a salt or cocrystal of Compound 1 is administered with methotrexate, cyclophosphamide, 5-fluorouracil, taxane, everolimus, paclitaxel (e.g., Abraxane®), lapatinib, trastuzumab (Herceptin®), pamidronate disodium, eribulin mesylate, everolimus, gemcitabine, palbociclib, ixabepilone, ado-trastuzumab emtansine (Kadcyla®), pertuzumab, thiotepa, aromatase inhibitors, exemestane, selective estrogen modulators, estrogen receptor antagonists, anthracyclines, emtansine, and/or pexidartinib to patients with metastatic breast cancer.

In certain embodiments, a solid form comprising a salt or cocrystal of Compound 1 is administered with temozolomide, doxorubicin (Adriamycin®), everolimus, fluorouracil (Adrucil®, 5-fluorouracil), or streptozocin (Zanosar®) to patients with neuroendocrine tumors.

In certain embodiments, a solid form comprising a salt or cocrystal of Compound 1 is administered with methotrexate, gemcitabine, cisplatin, cetuximab, 5 fluorouracil, bleomycin, docetaxel or carboplatin to patients with recurrent or metastatic head or neck cancer. In one embodiment, a solid form comprising a salt or cocrystal of Compound 1 provided herein is administered with cetuximab, to patients with head or neck cancer.

In certain embodiments, a solid form comprising a salt or cocrystal of Compound 1 is administered with gemcitabine, paclitaxel (Abraxane®), 5-fluorouracil, everolimus (Afinitor®), irinotecan, mitomycin C, sunitinib or erlotinib (Tarceva®) to patients with pancreatic cancer.

In certain embodiments, a solid form comprising a salt or cocrystal of Compound 1 is administered to patients with colon cancer in combination with Iressa®, erlotinib (Tarceva®), oxaliplatin (Eloxatin®), 5-fluorouracil, fluorouracil injection (Adrucil®), irinotecan (Camptosar®), capecitabine (Xeloda®), cetuximab (Erbitux®), ramucirumab (Cyramza®), panitumumab (Veetibix®), bevacizumab (Avastin®), leucovorin calcium (Wellcovorin®), trifluridine+tipiracil (Lonsurf®), regorafenib (Stivarga®), ziv-aflibercept (Zaltrap®), trametinib (Mekinist®), paclitaxel (Taxol®), and/or docetaxel (Taxotere®). In some embodiments, a solid form comprising a salt or cocrystal of Compound 1 provided herein is administered to patients with colon cancer in combination with an EGFR inhibitor (for example cetuximab or erlotinib) and/or a BRAF inhibitor (for example, sorafenib, dabrafenib, or vemurafenib).

In certain embodiments, a solid form comprising a salt or cocrystal of Compound 1 is administered with capecitabine, cetuximab, erlotinib, trametinib, and/or vemurafenib to patients with refractory colorectal cancer or patients who fail first line therapy or have poor performance in colon or rectal adenocarcinoma. In some embodiments, a solid form comprising a salt or cocrystal of Compound 1 provided herein is administered to patients with refractory colorectal cancer or patients who fail first line therapy or have poor performance in colon or rectal adenocarcinoma in combination with an EGFR inhibitor (for example cetuximab or erlotinib) and a BRAF inhibitor (for example, sorafenib, dabrafenib, or vemurafenib). In some embodiments, a solid form comprising a salt or cocrystal of Compound 1 provided herein is administered to patients with refractory colorectal cancer or patients who fail first line therapy or have poor performance in colon or rectal adenocarcinoma in combination with an anti-RSPO antibody.

In certain embodiments, a solid form comprising a salt or cocrystal of Compound 1 provided herein is administered in combination with fluorouracil, leucovorin, trametinib and/or irinotecan to patients with Stage Ma to IV colorectal cancer or to patients who have been previously treated for metastatic colorectal cancer. In some embodiments, a solid form comprising a salt or cocrystal of Compound 1 provided herein is administered to patients with Stage Ma to IV colorectal cancer or to patients who have been previously treated for metastatic colorectal cancer, in combination with an EGFR inhibitor (for example cetuximab or erlotinib) and a BRAF inhibitor (for example, sorafenib, dabrafenib, or vemurafenib). In certain embodiments, a solid form comprising a salt or cocrystal of Compound 1 provided herein is administered to patients with refractory colorectal cancer in combination with capecitabine, xeloda, trametinib, oxaliplatin and/or irinotecan. In some embodiments, a solid form comprising a salt or cocrystal of Compound 1provided herein is administered to patients with refractory colorectal cancer, in combination with an EGFR inhibitor (for example cetuximab or erlotinib) and a BRAF inhibitor (for example, sorafenib, dabrafenib, or vemurafenib). In certain embodiments, a solid form comprising a salt or cocrystal of Compound 1 provided herein is administered with capecitabine, trametinib, and/or irinotecan to patients with refractory colorectal cancer or to patients with unresectable or metastatic colorectal carcinoma. In some embodiments, a solid form comprising a salt or cocrystal of Compound 1 provided herein is administered to patients with refractory colorectal cancer or to patients with unresectable or metastatic colorectal carcinoma, in combination with an EGFR inhibitor (for example cetuximab or erlotinib) and a BRAF inhibitor (for example, sorafenib, dabrafenib, or vemurafenib).

In certain embodiments, a solid form comprising a salt or cocrystal of Compound 1 provided herein is administered alone or in combination with interferon alpha, 5-fluorouracil/leucovorin or capecitabine to patients with unresectable or metastatic hepatocellular carcinoma; or with cisplatin and thiotepa, or with sorafenib to patients with primary or metastatic liver cancer. In certain embodiments, a solid form comprising a salt or cocrystal of Compound 1 provided herein is administered alone or in combination with sorafenib, sunitinib, erlotinib, and/or sirolimus, to patients with unresectable or metastatic hepatocellular carcinoma; or with sorafenib, sunitinib, erlotinib, and/or sirolimus to patients with primary or metastatic liver cancer. In some embodiments, a solid form comprising a salt or cocrystal of Compound 1 provided herein is administered to patients with primary, unresectable, or metastatic liver cancer, in combination with an immune checkpoint inhibitor (for example, an anti-CTLA4, anti-PD1, anti-PD-L1, anti-Tim-3, or anti-Lag-3 antibody) or a BRAF inhibitor (for example, sorafenib, dabrafenib, or vemurafenib). In some such embodiments, the anti-PD-1 or anti-PD-L1 antibody is avelumab, durvalumab, MEDI0680, atezolizumab, BMS-936559, nivolumab, pembrolizumab, pidilizumab, or PDR-001. In certain embodiments, a solid form comprising a salt or cocrystal of Compound 1 provided herein is administered alone or in combination with lenalidomide, pomalidomide or CC122 to patients with primary, unresectable or metastatic hepatocellular carcinoma. In certain embodiments, a solid form comprising a salt or cocrystal of Compound 1 provided herein is administered alone or in combination CC223 to patients with primary, unresectable or metastatic hepatocellular carcinoma.

In certain embodiments, a solid form comprising a salt or cocrystal of Compound 1 provided herein is administered in combination with cisplatin/5-fluorouracil, ramucirumab (Cyramza®), docetaxel (Taxotere®), doxorubicin hydrochloride, fluorouracil injection, trastuzumab (Herceptin), mitomycin C (Mitozytrex®, Mutamycin®), and/or ramucirumab, to patients with gastric (stomach) cancer.

In certain embodiments, a solid form comprising a salt or cocrystal of Compound 1 provided herein is administered in combination with an immune checkpoint inhibitor (for example, an anti-CTLA4, anti-PD1, anti-PD-L1, anti-Tim-3, or anti-Lag-3 antibody) and/or a BRAF inhibitor (for example, sorafenib, dabrafenib, or vemurafenib) to patients with various types or stages of melanoma. In some embodiments, a solid form comprising a salt or cocrystal of Compound 1 provided herein is administered in combination with aldesleukin, cobimetinib, dabrafenib, dacarbazine, IL-2 (aldesleukin, Proleukin®), talimogene laherparepvec (Imlygic®), recombinant interferon alfa-2b (Intron® A), ipilimumab (Yervoy®), pembrolizumab (Keytruda®), lapatinib, trametinib (Mekinist®), nivolumab (Opdivo®), peginterferon alfa-2b (Sylatron®), dabrafenib (Tafinlar®), and/or vemurafenib (Zelboraf®), to patients with various types or stages of melanoma.

In certain embodiments, a solid form comprising a salt or cocrystal of Compound 1 is administered in combination with doxorubicin, paclitaxel, vinblastine or pegylated interferon alpha to patients with Kaposi's sarcoma.

In certain embodiments, a solid form comprising a salt or cocrystal of Compound 1 is administered in combination with fludarabine, carboplatin, and/or topotecan to patients with refractory or relapsed or high-risk acuted myelogenous leukemia.

In certain embodiments, a solid form comprising a salt or cocrystal of Compound 1 is administered in combination with liposomal daunorubicin, topotecan and/or cytarabine to patients with unfavorable karotype acute myeloblastic leukemia.

In certain embodiments, a solid form comprising a salt or cocrystal of Compound 1 is administered in combination with methotrexate, mechlorethamine hydrochloride, afatinib dimaleate, pemetrexed, bevacizumab, carboplatin, cisplatin, ceritinib, crizotinib, ramucirumab, pembrolizumab, docetaxel, vinorelbine tartrate, gemcitabine, protein-bound paclitaxel (Abraxane®), erlotinib, geftinib, and/or irinotecan to patients with non-small cell lung cancer.

In certain embodiments, a solid form comprising a salt or cocrystal of Compound 1 provided herein is administered with docetaxel (Taxotere^{c}) to patients with non-small cell lung cancer who have been previously treated with carboplatin/etoposide and radiotherapy.

In certain embodiments, a solid form comprising a salt or cocrystal of Compound 1 provided herein is administered in combination with carboplatin and/or taxotere, or in combination with carboplatin, pacilitaxel and/or thoracic radiotherapy to patients with non-small cell lung cancer.

In certain embodiments, a solid form comprising a salt or cocrystal of Compound 1 provided herein is administered in combination with taxotere to patients with stage IIIB or IV non-small cell lung cancer.

In certain embodiments, a solid form comprising a salt or cocrystal of Compound 1 provided herein is administered in combination with oblimersen (Genasense), methotrexate, mechlorethamine hydrochloride, etoposide, topotecan or doxorubicin to patients with small cell lung cancer.

In certain embodiments, a solid form comprising a salt or cocrystal of Compound 1 provided herein and doxetaxol are administered to patients with small cell lung cancer who were previously treated with carbo/VP 16 and radiotherapy.

In certain embodiments, a solid form comprising a salt or cocrystal of Compound 1 provided herein is administered to patients with various types or stages of ovarian cancer such as peritoneal carcinoma, papillary serous carcinoma, refractory ovarian cancer or recurrent ovarian cancer, in combination with taxol, carboplatin, doxorubicin, gemcitabine, cisplatin, xeloda, paclitaxel, dexamethasone, avastin, cyclophosphamide, topotecan, olaparib, thiotepa, or a combination thereof.

In certain embodiments, a solid form comprising a salt or cocrystal of Compound 1 provided herein is administered to patients with various types or stages of prostate cancer, in combination with capecitabine (Xeloda®), 5-fluorouracil+leucovorin, gemcitabine, irinotecan+gemcitabine, cyclophosphamide, vincristine, dexamethasone, granulocyte macrophage colony-stimulating factor (GM-CSF), celecoxib, ganciclovir, paclitaxel, adriamycin, docetaxel, estramustine, estramustine (Emcyt®), denderon, abiraterone (Zytiga®), bicalutamide, cabazitaxel, degarelix, enzalutamide, goserelin (Zoladex®), leuprolide acetate, mitoxantrone hydrochloride, prednisone, sipuleucel-T, radium 223 dichloride, or a combination thereof.

In certain embodiments, a solid form comprising a salt or cocrystal of Compound 1 provided herein is administered to patients with various types or stages of renal cell cancer, in combination with capecitabine, IFN, tamoxifen, IL-2, GM-C SF, celecoxib, or a combination thereof.

In certain embodiments, a solid form comprising a salt or cocrystal of Compound 1 provided herein is administered to patients with various types or stages of gynecologic, uterus or soft tissue sarcoma cancers in combination with IFN, dactinomycin, doxorubicin, imatinib mesylate, pazopanib, hydrochloride, trabectedin, a COX-2 inhibitor such as celecoxib (Celebrex®), and/or sulindac.

In certain embodiments, a solid form comprising a salt or cocrystal of Compound 1 provided herein is administered to patients with various types or stages of solid tumors in combination with celecoxib, etoposide, cyclophosphamide, docetaxel, apecitabine, IFN, tamoxifen, IL-2, GM-CSF, or a combination thereof.

In certain embodiments, a solid form comprising a salt or cocrystal of Compound 1 provided herein is administered alone or in combination with vinorelbine to patients with malignant mesothelioma, or stage IIIB non-small cell lung cancer with pleural implants or malignant mesothelioma syndrome.

In certain embodiments, a solid form comprising a salt or cocrystal of Compound 1 is administered in combination with navitoclax, venetoclax and/or obatoclax to patients with lymphoma and other blood cancers.

In certain embodiments, a solid form comprising a salt or cocrystal of Compound 1 provided herein is administered in combination with arsenic trioxide, fludarabine, carboplatin, daunorubicin, cyclophosphamide, cytarabine, doxorubicin, idarubicin, mitoxantrone hydrochloride, thioguanine, vincristine, and/or topotecan to patients with acute myeloid leukemia, including refractory or relapsed or high-risk acute myeloid leukemia.

In certain embodiments, a solid form comprising a salt or cocrystal of Compound 1 provided herein is administered in combination with liposomal daunorubicin, topotecan and/or cytarabine to patients with unfavorable karotype acute myeloblastic leukemia.

In certain embodiments, a solid form comprising a salt or cocrystal of Compound 1 is administered alone or in combination with a second active ingredient such as vinblastine or fludarabine adcetris, ambochlorin, becenum, bleomycin, brentuximab vedotin, carmustine, chlorambucil, cyclophosphamide, dacarbazine, doxorubicin, lomustine, mechlorethamine hydrochloride, prednisone, procarbazine hydrochloride (Matulane®), or vincristine to patients with various types of lymphoma, including, but not limited to, Hodgkin's lymphoma, non-Hodgkin's lymphoma, cutaneous T-Cell lymphoma, cutaneous B-Cell lymphoma, diffuse large B-Cell lymphoma or relapsed or refractory low grade follicular lymphoma.

In certain embodiments, a solid form comprising a salt or cocrystal of Compound 1 provided herein is administered to patients with various types or stages of multiple myeloma in combination with dexamethasone, zoledronic acid, palmidronate, GM-CSF, clarithromycin (Biaxin®), vinblastine, melphalan, busulphan, cyclophosphamide, IFN, prednisone, bisphosphonate, celecoxib, arsenic trioxide, peginterferon alfa-2b, vincristine, Carmustine (Becenum®), bortezomib, carfilzomib, doxorubicin, panobinostat, lenalidomide, pomalidomide, thalidomide, plerixafor (Mozobil®) or a combination thereof.

In certain embodiments, a solid form comprising a salt or cocrystal of Compound 1 provided herein is administered to patients with various types or stages of multiple myeloma in combination with chimeric antigen receptor (CAR) T-cells.

In certain embodiments, a solid form comprising a salt or cocrystal of Compound 1 provided herein is administered to patients with relapsed or refractory multiple myeloma in combination with doxorubicin (Doxil®), vincristine and/or dexamethasone (Decadron®).

In certain embodiments, a solid form comprising a salt or cocrystal of Compound 1 provided herein is administered to patients with scleroderma or cutaneous vasculitis in combination with celecoxib (Celebrex®), etoposide, cyclophosphamide, docetaxel, capecitabine, IFN, tamoxifen, IL-2, GM-CSF, or a combination thereof.

Also encompassed herein is a method of increasing the dosage of an anti-cancer drug or agent that can be safely and effectively administered to a patient, which comprises administering to the patient (e.g., a human) a solid form comprising a salt or cocrystal of Compound 1. Patients that can benefit by this method are those likely to suffer from an adverse effect associated with anti-cancer drugs for treating a specific cancer of the skin, subcutaneous tissue, lymph nodes, brain, lung, liver, bone, intestine, colon, heart, pancreas, adrenal, kidney, prostate, breast, colorectal, or combinations thereof. The administration of a solid form comprising a salt or cocrystal of Compound 1 alleviates or reduces adverse effects which are of such severity that it would otherwise limit the amount of anti-cancer drug.

In one embodiment, a solid form comprising a salt or cocrystal of Compound 1 is administered orally and daily in an amount ranging from about 0.1 to about 150 mg, from about 1 to about 50 mg, or from about 2 to about 25 mg, prior to, during, or after the occurrence of the adverse effect associated with the administration of an anti-cancer drug to a patient. In certain embodiments, a solid form comprising a salt or cocrystal of Compound 1 is administered in combination with specific agents such as heparin, aspirin, coumadin, or G-CSF to avoid adverse effects that are associated with anti-cancer drugs such as but not limited to neutropenia or thrombocytopenia.

In one embodiment, a solid form comprising a salt or cocrystal of Compound 1 is administered to patients with diseases and disorders associated with or characterized by, undesired angiogenesis in combination with additional active ingredients, including, but not limited to, anti-cancer drugs, anti-inflammatories, antihistamines, antibiotics, and steroids.

In another embodiment, encompassed herein is a method of treating, preventing and/or managing cancer, which comprises administering the solid form comprising a salt or cocrystal of Compound 1 in conjunction with (e.g. before, during, or after) conventional therapy including, but not limited to, surgery, immunotherapy, biological therapy, radiation therapy, or other non-drug based therapy presently used to treat, prevent or manage cancer. The combined use of the compound provided herein and conventional therapy may provide a unique treatment regimen that is unexpectedly effective in certain patients. Without being limited by theory, it is believed that the solid form comprising a salt or cocrystal of Compound 1 may provide additive or synergistic effects when given concurrently with conventional therapy.

In another embodiment, the method of treatment provided herein comprises the administration of a second agent that is an anti-inflammatory drug, e.g., a steroidal anti-inflammatory drug, or a non-steroidal anti-inflammatory drug (NSAID), acetaminophen, naproxen, ibuprofen, acetylsalicylic acid, and the like. In a more specific embodiment in which an NSAID is administered, a proton pump inhibitor (PPI), e.g., omeprazole may also administered. In one embodiment, the antiinflammatory agent is a corticosteroid. In another embodiment, the antiinflammatory agent is colchicine.

In another embodiment, the second therapeutic agent is an immunomodulatory compound or an immunosuppressant compound such as azathioprine (Imuran™, Azasan™), methotrexate (Rheumatrex™, Trexall™), penicillamine (Depen™, Cuprimine™), cyclophosphamide (Cytoxan™), mycophenalate (CellCept™, Myfortic™), bosentan (Tracleer®), prednisone (Deltasone™, Liquid Pred™), and a PDE5 inhibitor. In another embodiment, where the affected individual has digital ulcerations and pulmonary hypertension, a vasodilator such as prostacyclin (iloprost) may be administered.

In another embodiment, the second therapeutic agent is an inhibitor of ActRII receptors or an activin-ActRII inhibitor. Inhibitors of ActRII receptors include ActRIIA inhibitors and ActRIIB inhibitors. Inhibitors of ActRII receptors can be polypeptides comprising activin-binding domains of ActRII. In certain embodiments, the activin-binding domain comprising polypeptides are linked to an Fc portion of an antibody (i.e., a conjugate comprising an activin-binding domain comprising polypeptide of an ActRII receptor and an Fc portion of an antibody is generated). In certain embodiments, the activin-binding domain is linked to an Fc portion of an antibody via a linker, e.g., a peptide linker.

Examples of non-antibody proteins selected for activin or ActRIIA binding and methods for design and selection of the same are found in WO/2002/088171, WO/2006/055689, WO/2002/032925, WO/2005/037989, US 2003/0133939, and US 2005/0238646, each of which is incorporated herein by reference in its entirety.

As discussed elsewhere herein, encompassed herein is a method of reducing, treating and/or preventing adverse or undesired effects associated with conventional therapy including, but not limited to, surgery, chemotherapy, radiation therapy, hormonal therapy, biological therapy and immunotherapy. A solid form comprising a salt or cocrystal of Compound 1 provided herein and other active ingredient can be administered to a patient prior to, during, or after the occurrence of the adverse effect associated with conventional therapy.

Use With Transplantation Therapy

A solid form comprising a salt or cocrystal of Compound 1 provided herein can be used to reduce the risk of Graft Versus Host Disease (GVHD). Therefore, encompassed herein is a method of treating, preventing and/or managing cancer, which comprises administering a solid form comprising a salt or cocrystal of Compound 1 in conjunction with transplantation therapy.

As those of ordinary skill in the art are aware, the treatment of cancer is often based on the stages and mechanism of the disease. For example, as inevitable leukemic transformation develops in certain stages of cancer, transplantation of peripheral blood stem cells, hematopoietic stem cell preparation or bone marrow may be necessary. The combined use of a solid form comprising a salt or cocrystal of Compound 1 provided herein and transplantation therapy provides a unique and unexpected synergism. In particular, a solid form comprising a salt or cocrystal of Compound 1 exhibits immunomodulatory activity that may provide additive or synergistic effects when given concurrently with transplantation therapy in patients with cancer.

A solid form comprising a salt or cocrystal of Compound 1 can work in combination with transplantation therapy reducing complications associated with the invasive procedure of transplantation and risk of GVHD. Encompassed herein is a method of treating, preventing and/or managing cancer which comprises administering to a patient (e.g., a human) a solid form comprising a salt or cocrystal of Compound 1, before, during, or after the transplantation of umbilical cord blood, placental blood, peripheral blood stem cell, hematopoietic stem cell preparation, or bone marrow. Some examples of stem cells suitable for use in the methods provided herein are disclosed in U.S. Pat. No. 7,498,171, the disclosure of which is incorporated herein by reference in its entirety.

In one embodiment, a solid form comprising a salt or cocrystal of Compound 1 is administered to patients with multiple myeloma before, during, or after the transplantation of autologous peripheral blood progenitor cell.

In another embodiment, a solid form comprising a salt or cocrystal of Compound 1 is administered to patients with relapsing multiple myeloma after the stem cell transplantation.

In yet another embodiment, a solid form comprising a salt or cocrystal of Compound 1 and prednisone are administered as maintenance therapy to patients with multiple myeloma following the transplantation of autologous stem cell.

In yet another embodiment, a solid form comprising a salt or cocrystal of Compound 1 and dexamethasone are administered as salvage therapy for low risk post transplantation to patients with multiple myeloma.

In yet another embodiment, a solid form comprising a salt or cocrystal of Compound 1 and dexamethasone are administered as maintenance therapy to patients with multiple myeloma following the transplantation of autologous bone marrow.

In yet another embodiment, a solid form comprising a salt or cocrystal of Compound 1 is administered following the administration of high dose of melphalan and the transplantation of autologous stem cell to patients with chemotherapy responsive multiple myeloma.

In yet another embodiment, a solid form comprising a salt or cocrystal of Compound 1 and PEG INTRO-A are administered as maintenance therapy to patients with multiple myeloma following the transplantation of autologous CD34-selected peripheral stem cell.

In yet another embodiment, a solid form comprising a salt or cocrystal of Compound 1 is administered with post transplant consolidation chemotherapy to patients with newly diagnosed multiple myeloma to evaluate anti-angiogenesis.

In still another embodiment, a solid form comprising a salt or cocrystal of Compound 1 and dexamethasone are administered as maintenance therapy after DCEP consolidation, following the treatment with high dose of melphalan and the transplantation of peripheral blood stem cell to 65 years of age or older patients with multiple myeloma.

In one embodiment, a solid form comprising a salt or cocrystal of Compound 1 is administered to patients with NHL (e.g., DLBCL) before, during, or after the transplantation of autologous peripheral blood progenitor cell.

In another embodiment, a solid form comprising a salt or cocrystal of Compound 1 is administered to patients with NHL (e.g., DLBCL) after a stem cell transplantation.

Cycling Therapy

In certain embodiments, the prophylactic or therapeutic agents provided herein are cyclically administered to a patient. Cycling therapy involves the administration of an active agent for a period of time, followed by a rest for a period of time, and repeating this sequential administration. Cycling therapy can reduce the development of resistance to one or more of the therapies, avoid, or reduce the side effects of one of the therapies, and/or improves the efficacy of the treatment.

In one aspect, the cycling therapy includes an administration of a solid form comprising a salt or cocrystal of Compound 1 for a period of 5 days followed by a 2 day rest period. In still another aspect, the cycling therapy includes an extended administration period followed by a 7 day rest period. An “extended administration period” as used herein refers to continual daily administration (e.g., QD) of a solid form comprising a salt or cocrystal of Compound 1 for 7 or more days. In certain embodiments, an extended administration period includes continual daily administration of a solid form comprising a salt or cocrystal of Compound 1 for 7, 8, 9, 10, 11, 12, 13, 14, 15, 16, 17, 18, 19, 20, or 21 days. In certain embodiments, an extended administration period includes continual daily administration of a solid form comprising a salt or cocrystal of Compound 1 for 10, 11, 12, 13, 14, 15, 16, 17, 18, 19, 20, or 21 days. In one embodiment, the continual administration is for 7 days. In one embodiment, the continual administration is for 8 days. In one embodiment, the continual administration is for 9 days. In one embodiment, the continual administration is for 10 days. In one embodiment, the continual administration is for 11 days. In one embodiment, the continual administration is for 12 days. In one embodiment, the continual administration is for 13 days. In one embodiment, the continual administration is for 14 days. In one embodiment, the continual administration is for 15 days. In one embodiment, the continual administration is for 16 days. In one embodiment, the continual administration is for 17 days. In one embodiment, the continual administration is for 18 days. In one embodiment, the continual administration is for 19 days. In one embodiment, the continual administration is for 20 days. In one embodiment, the continual administration is for 21 days.

In another embodiment the cycling therapy includes an administration period of at least 7 continual days of administration. The administration period can be 21 days, where the administration period is repeated for at least 1 more cycles as described herein.

Cycling therapies can be repeated for at least 2, 3, 4, 5, 6, 7, 8, or more cycles. In certain instances a cycling therapy includes from one to about 24 cycles, from about two to about 16 cycles, or from about two to about four cycles. In certain instances, the cycling therapy is not limited to the number of cycles, and the therapy is continued until diease progression. Cycles, can in certain instances, include varying the duration of administration periods and/or rest periods described herein.

In one aspect is a cycling therapy that includes administering a solid form comprising a salt or cocrystal of Compound 1 as described herein in an administration period of 5 days followed by a 2 day rest period (e.g., a 5/7 cycling therapy). In one embodiment, the 5/7 cycling therapy is repeated from about 2 to about 16 cycles. In one embodiment, the 5/7 cycling therapy is repeated 2, 3, or 4 cycles. The dosage amound of Compound 1 in the 5/7 cycling therapy is as described herein. In one embodiment the 5/7 cycling therapy includes administering a solid form comprising a salt or cocrystal of Compound 1 at a dosage amount of about 0.1 mg to about 20 mg, from about 0.1 mg to about 15 mg, from about 0.1 mg to about 10 mg, from about 1 mg to about 7 mg, from about 1 mg to about 5 mg, from about 1 mg to about 4 mg, or from about 1 mg to about 3 mg.

In one embodiment the 5/7 cycling therapy includes administering a solid form comprising a salt or cocrystal of Compound 1 at a dosage amount of about 1 mg, 2 mg, 3 mg, 4 mg or 5 mg.

In certain embodiments, the amount of a solid form comprising a salt or cocrystal of Compound ladministered in the 5/7 cycling therapy is greater than an amount administered in an extended administration period described herein. Without being bound by any particular theory, a higher dosage administrat ion may be more tolerable to a patient and result in greater efficacy over the shortened administration period.

In other embodiments, the amount of a solid form comprising a salt or cocrystal of Compound 1 administered in a 5/7 cycling therapy described herein is less than an amount administered in an extended administration period described herein. Such lowered administration can be performed over any number of cycles described herein and in particular over a number of cycles greater than 1, 3, 5, 7, or 10 cycles. In certain embodiments, the lower amount of administered compound allows for decreased observation of development of resistance to the compound.

In another aspect is cycling therapy that includes administering a solid form comprising a salt or cocrystal of Compound 1 as described herein in an extended administration period as described herein followed by a 7 day rest period. In one embodiment the extended administration period includes administering a solid form comprising a salt or cocrystal of Compound 1 as described herein daily over 21 continual days. In one embodiment the cycling therapy includes an extended administration period is repeated consecutively over 3, 4, 5, 6, 7, 8, 9, or more cycles.

In one embodiment, a cycling therapy that includes an extended administration period described herein includes administration of a solid form comprising a salt or cocrystal of Compound 1 daily at a dosage amount of about 0.1 mg to about 20 mg, from about 0.1 mg to about 15 mg, from about 0.1 mg to about 10 mg, from about 1 mg to about 7 mg, from about 1 mg to about 5 mg, from about 1 mg to about 4 mg, or from about 1 mg to about 3 mg.

In one embodiment, the amount of compound administered in a cycling therapy that includes an extended administration period described herein is 3 or 4 mg. In one embodiment, the amount of compound administered in a cycling therapy that includes an extended administration period described herein is from about 3 mg to about 4 mg.

In yet another aspect is a cycling therapy that includes one or more of the cycling therapies described herein. Thus in one embodiment, a cycling therapy includes at least one 5/7 cycle as set forth above and at least one cycling therapy that includes an extended administration period. A solid form comprising a salt or cocrystal of Compound 1 can be administered at the same amount for all administration periods in such cycling therapies and can be administered as described herein. Alternatively, in one embodiment, a solid form comprising a salt or cocrystal of Compound 1 is administered at different doses between the administration periods (e.g., a 5 day administration period and an extended administration period described herein). In one such embodiment, a solid form comprising a salt or cocrystal of Compound 1 is administered at an amount lower in a second administration period than a first administration period as described herein. A solid form comprising a salt or cocrystal of Compound 1 can be administered according to the dosages and dosage amounts described herein.

Cyclic therapies that include administration periods of varying lengths as set forth above can be administered in any order and independently in any number of cycles described herein. Thus, in one embodiment is a cycling therapy of a solid form comprising a salt or cocrystal of Compound 1 that includes at least 1, 2, 3, 4, 5, 6 or more 5/7 cycling therapies as described herein and 1, 2, 3, 4, 5, 6 or more cycling therapies that include an extended administration period as described herein, where the two therapies can be administered in any combination (e.g., two 5/7 cycling therapies followed by 1 cycling therapy that includes an extended administration period.)

Provided herein in one embodiment is a method of treating, preventing, managing, and/or ameliorating cancer while reducing an adverse effect associated with such treatment, prevention, management, or amelioration by administering to a subject in need thereof an effective amount of a solid form comprising a salt or cocrystal of Compound 1 where the compound is administered to the subject in a cycling therapy that includes (a) an administration period of 5 days followed by a rest period of 2 days or (b) an extended administration period of 21 days as described herein followed by a rest period of 7 days, and where the cycling therapy is administered to the patient for a period of at least 1 cycle.

In another embodiment, a solid form comprising a salt or cocrystal of Compound 1 and a second active ingredient are administered orally, with administration of the solid form comprising a salt or cocrystal of Compound 1 occurring 30 to 60 minutes prior to a second active ingredient, during a cycle of four to six weeks. In certain embodiments, the combination of a solid form comprising a salt or cocrystal of Compound 1 and a second active ingredient is administered by intravenous infusion over about 90 minutes every cycle. In certain embodiments, one cycle comprises the administration from about 0.1 to about 150 mg/day of a solid form comprising a salt or cocrystal of Compound 1 and from about 50 to about 200 mg/m²/day of a second active ingredient daily for three to four weeks and then one or two weeks of rest. In certain embodiments, the number of cycles during which the combinatorial treatment is administered to a patient is ranging from about one to about 24 cycles, from about two to about 16 cycles, or from about four to about three cycles.

### 5.4. PHARMACEUTICAL COMPOSITIONS

Pharmaceutical compositions and single unit dosage forms comprising one or more solid forms comprising Compound 1 or a pharmaceutically acceptable salt thereof are provided herein. Also provided herein are methods for preparing pharmaceutical compositions and single unit dosage forms comprising one or more solid forms comprising Compound 1 or a pharmaceutically acceptable salt thereof. For example, in certain embodiments, individual dosage forms comprising a solid form provided herein or prepared using solid form provided herein may be suitable for oral, mucosal (including rectal, nasal, or vaginal), parenteral (including subcutaneous, intramuscular, bolus injection, intraarterial, or intravenous), sublingual, transdermal, buccal, or topical administration.

In certain embodiments, pharmaceutical compositions and dosage forms provided herein comprise one or more solid forms comprising Compound 1 or a pharmaceutically acceptable salt thereof. Certain embodiments herein provide pharmaceutical compositions and dosage forms comprising a solid form comprising a salt or cocrystal of Compound 1, such as, e.g., Form A of a sulfate salt of Compound 1, Form A or Form B of a mesylate salt of Compound 1, Form A or Form B of an esylate salt of Compound 1, Form A of a besylate salt of Compound 1, Form A of a napadisylate salt of Compound 1, Form A of a napsilate salt of Compound 1, Form A or Form B of a thiocyanate salt of Compound 1, and Form A or Form B of a tosylate salt of Compound 1; Compound 1 acetic acid cocrystal Form A, Compound 1 isethionic acid cocrystal Form A, Compound 1 isethionic acid cocrystal Form B, Compound 1 lauric acid cocrystal Form A, Compound 1 maleic acid cocrystal Form A, Compound 1 phosphoric acid cocrystal Form A as provided herein.

Certain embodiments herein provide pharmaceutical compositions and dosage forms comprising a mixture of solid forms comprising Compound 1, including, e.g., a mixture comprising one or more of the following: Form A of a sulfate salt of Compound 1, Form A or Form B of a mesylate salt of Compound 1, Form A or Form B of an esylate salt of Compound 1, Form A of a besylate salt of Compound 1, Form A of a napadisylate salt of Compound 1, Form A of a napsilate salt of Compound 1, Form A or Form B of a thiocyanate salt of Compound 1, and Form A or Form B of a tosylate salt of Compound 1; Compound 1 acetic acid cocrystal Form A, Compound 1 isethionic acid cocrystal Form A, Compound 1 isethionic acid cocrystal Form B, Compound 1 lauric acid cocrystal Form A, Compound 1 maleic acid cocrystal Form A, Compound 1 phosphoric acid cocrystal Form A as provided herein.

Pharmaceutical compositions and dosage forms provided herein typically also comprise one or more pharmaceutically acceptable excipient, diluent or carrier.

A particular pharmaceutical composition encompassed by this embodiment comprises one or more solid forms comprising Compound 1 or a pharmaceutically acceptable salt thereof and at least one additional therapeutic agent. Examples of additional therapeutic agents include, but are not limited to: anti-cancer drugs and anti-inflammation therapies including, but not limited to, those provided herein.

Single unit dosage forms of the disclosure are suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the disclosure will typically vary depending on their use. For example, a dosage form used in the acute treatment of inflammation or a related disorder may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease or disorder. These and other ways in which specific dosage forms encompassed by this disclosure will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form.

Lactose-free compositions of the disclosure can comprise excipients that are well known in the art and are listed, for example, in the U.S. Pharmocopia (USP) SP (XXI)/NF (XVI). In general, lactose-free compositions comprise an active ingredient, a binder/filler, and a lubricant in pharmaceutically compatible and pharmaceutically acceptable amounts. Preferred lactose-free dosage forms comprise an active ingredient, microcrystalline cellulose, pre-gelatinized starch, and magnesium stearate.

This disclosure further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the disclosure can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The disclosure further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms provided herein lie within the range of from about 1 mg to about 1,000 mg per day, given as a single once-a-day dose in the morning but preferably as divided doses throughout the day. More specifically, the daily dose is administered twice daily in equally divided doses. Specifically, a daily dose range may be from about 5 mg to about 500 mg per day, more specifically, between about 10 mg and about 200 mg per day. In managing the patient, the therapy may be initiated at a lower dose, perhaps about 1 mg to about 25 mg, and increased if necessary up to about 200 mg to about 1,000 mg per day as either a single dose or divided doses, depending on the patient's global response.

### 5.4.1. ORAL DOSAGE FORMS

Pharmaceutical compositions of the disclosure that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally Remington's Pharmaceutical Sciences, 18th ed., Mack Publishing, Easton Pa. (1990).

Typical oral dosage forms of the disclosure are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the disclosure include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the disclosure is typically present in from about 50 to about 99 weight percent of the pharmaceutical composition or dosage form.

Suitable forms of microcrystalline cellulose include, but are not limited to, the materials sold as AVICEL-PH-101™, AVICEL-PH-103™, AVICEL RC-581™, AVICEL-PH-105™ (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC-581™. Suitable anhydrous or low moisture excipients or additives include AVICEL-PH-103™ and Starch 1500 LM™.

Disintegrants are used in the compositions of the disclosure to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the disclosure. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms of the disclosure include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200™, manufactured by W.R. Grace Co. of Baltimore, Md.), a coagulated aerosol of synthetic silica (marketed by Degussa Co. of Plano, Tex.), CAB-O-SIL™ (a pyrogenic silicon dioxide product sold by Cabot Co. of Boston, Mass.), and mixtures thereof. If used at all, lubricants are typically used in an amount of less than about one weight percent of the pharmaceutical compositions or dosage forms into which they are incorporated.

### 5.4.2. DELAYED RELEASE DOSAGE FORMS

Solid forms comprising Compound 1 as provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the disclosure. The disclosure thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### 5.4.3. PARENTERAL DOSAGE FORMS

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the disclosure are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the disclosure.

### 5.4.4. TRANSDERMAL, TOPICAL, AND MUCOSAL DOSAGE FORMS

Transdermal, topical, and mucosal dosage forms of the disclosure include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this disclosure are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

Depending on the specific tissue to be treated, additional components may be used prior to, in conjunction with, or subsequent to treatment with active ingredients of the disclosure. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80™ (polysorbate 80) and Span 60™ (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different solid forms comprising the active ingredients can be used to further adjust the properties of the resulting composition.

### 5.4.5. KITS

This disclosure encompasses kits which, when used by the medical practitioner, can simplify the administration of appropriate amounts of active ingredients to a patient.

A typical kit of the disclosure comprises a unit dosage form of Compound 1, or a pharmaceutically acceptable solid form or prodrug thereof, and a unit dosage form of a second active ingredient. Examples of second active ingredients include, but are not limited to, those listed herein.

Kits of the disclosure can further comprise devices that are used to administer the active ingredient(s). Examples of such devices include, but are not limited to, syringes, drip bags, patches, and inhalers.

Kits of the disclosure can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 6. EXAMPLES

### 6.1. EXAMPLE 1

### Assays

### 6.1.1. TNFα Inhibition Assay in PMBC

Peripheral blood mononuclear cells (PBMC) from normal donors are obtained by Ficoll Hypaque (Pharmacia, Piscataway, N.J., USA) density centrifugation. Cells are cultured in RPMI 1640 (Life Technologies, Grand Island, N.Y., USA) supplemented with 10% AB+human serum (Gemini Bio-products, Woodland, Calif., USA), 2 mM L-glutamine, 100 U/mL penicillin, and 100 μg/mL streptomycin (Life Technologies).

PBMC (2×10⁵ cells) are plated in 96-well flat-bottom Costar tissue culture plates (Corning, N.Y., USA) in triplicate. Cells are stimulated with LPS (from Salmonella abortus equi, Sigma cat. no. L-1887, St. Louis, Mo., USA) at 1 ng/mL final in the absence or presence of compounds. Compounds provided herein are dissolved in DMSO (Sigma) and further dilutions are done in culture medium immediately before use. The final DMSO concentration in all assays can be about 0.25%. Compounds are added to cells 1 hour before LPS stimulation. Cells are then incubated for 18-20 hours at 37° C. in 5% CO₂, and supernatants are then collected, diluted with culture medium and assayed for TNFα levels by ELISA (Endogen, Boston, Mass., USA). IC₅₀s are calculated using non-linear regression, sigmoidal dose-response, constraining the top to 100% and bottom to 0%, allowing variable slope (GraphPad Prism v3.02). In two experiments, Compound 1 demonstrated an IC50 of 10 and 85 nM.

### 6.1.2. IL-2 and MIP-3α Production by T Cells

PBMC are depleted of adherent monocytes by placing 1×10⁸ PBMC in 10 mL complete medium (RPMI 1640 supplemented with 10% heat-inactivated fetal bovine serum, 2 mM L-glutamine, 100 U/mL penicillin, and 100 μg/mL streptomycin) per 10 cm tissue culture dish, in 37° C., 5% CO₂ incubator for 30-60 minutes. The dish is rinsed with medium to remove all non-adherent PBMC. T cells are purified by negative selection using the following antibody (Pharmingen) and Dynabead (Dynal) mixture for every 1×10⁸ non-adherent PBMC: 0.3 mL Sheep anti-mouse IgG beads, 15 μL anti-CD16, 15 μL anti-CD33, 15 μL anti-CD56, 0.23 mL anti-CD19 beads, 0.23 mL anti-HLA class II beads, and 56 μL anti-CD14 beads. The cells and bead/antibody mixture is rotated end-over-end for 30-60 minutes at 4° C. Purified T cells are removed from beads using a Dynal magnet. Typical yield is about 50% T cells, 87-95% CD3⁺ by flow cytometry.

Tissue culture 96-well flat-bottom plates are coated with anti-CD3 antibody OKT3 at 5 μg/mL in PBS, 100 μL per well, incubated at 37° C. for 3-6 hours, then washed four times with complete medium 100 μL/well just before T cells are added. Compounds are diluted to 20 times of final in a round bottom tissue culture 96-well plate. Final concentrations are about 10 μM to about 0.00064 μM. A 10 mM stock of compounds provided herein is diluted 1:50 in complete for the first 20× dilution of 200 μM in 2% DMSO and serially diluted 1:5 into 2% DMSO. Compound is added at 10 μl per 200 μl culture, to give a final DMSO concentration of 0.1%. Cultures are incubated at 37° C., 5% CO₂ for 2-3 days, and supernatants analyzed for IL-2 and MIP-3a by ELISA (R&D Systems). IL-2 and MIP-3α levels are normalized to the amount produced in the presence of an amount of a compound provided herein, and EC₅₀s calculated using non-linear regression, sigmoidal dose-response, constraining the top to 100% and bottom to 0%, allowing variable slope (GraphPad Prism v3.02).

### 6.1.3. Cell Proliferation Assay

Cell lines Namalwa, MUTZ-5, and UT-7 are obtained from the Deutsche Sammlung von Mikroorganismen and Zellkulturen GmbH (Braunschweig, Germany). The cell line KG-1 is obtained from the American Type Culture Collection (Manassas, Va., USA). Cell proliferation as indicated by ³H-thymidine incorporation is measured in all cell lines as follows.

Cells are plated in 96-well plates at 6000 cells per well in media. The cells are pre-treated with compounds at about 100, 10, 1, 0.1, 0.01, 0.001, 0.0001 and 0 μM in a final concentration of about 0.25% DMSO in triplicate at 37° C. in a humidified incubator at 5% CO₂ for 72 hours. One microcurie of ³H-thymidine (Amersham) is then added to each well, and cells are incubated again at 37° C. in a humidified incubator at 5% CO₂ for 6 hours. The cells are harvested onto UniFilter GF/C filter plates (Perkin Elmer) using a cell harvester (Tomtec), and the plates are allowed to dry overnight.

Microscint 20 (Packard) (25 μL/well) is added, and plates are analyzed in TopCount NXT (Packard). Each well is counted for one minute. Percent inhibition of cell proliferation is calculated by averaging all triplicates and normalizing to the DMSO control (0% inhibition). Each compound is tested in each cell line in three separate experiments. Final IC5os are calculated using non-linear regression, sigmoidal dose-response, constraining the top to 100% and bottom to 0%, allowing variable slope. (GraphPad Prism v3.02).

### 6.1.4. Immunoprecipitation and Immunoblot

Namalwa cells are treated with DMSO or an amount of a compound provided herein for 1 hour, then stimulated with 10 U/mL of Epo (R&D Systems) for 30 minutes. Cell lysates are prepared and either immunoprecipitated with Epo receptor Ab or separated immediately by SDS-PAGE. Immunoblots are probed with Akt, phospo-Akt (Ser473 or Thr308), phospho-Gabl (Y627), Gabl, IRS2, actin and IRF-1 Abs and analyzed on a Storm 860 Imager using ImageQuant software (Molecular Dynamics).

### 6.1.5. Cell Cycle Analysis

Cells are treated with DMSO or an amount of a compound provided herein overnight. Propidium iodide staining for cell cycle is performed using CycleTEST PLUS (Becton Dickinson) according to manufacturer's protocol. Following staining, cells are analyzed by a FACSCalibur flow cytometer using ModFit LT software (Becton Dickinson).

### 6.1.6. Apoptosis Analysis

Cells are treated with DMSO or an amount of a compound provided herein at various time points, then washed with annexin-V wash buffer (BD Biosciences). Cells are incubated with annexin-V binding protein and propidium iodide (BD Biosciences) for 10 minutes. Samples are analyzed using flow cytometry.

### 6.1.7. Luciferase Assay

Namalwa cells are transfected with 4 μg of AP1-luciferase (Stratagene) per 1×10⁶ cells and 3 μL Lipofectamine 2000 (Invitrogen) reagent according to manufacturer's instructions. Six hours post-transfection, cells are treated with DMSO or an amount of a compound provided herein. Luciferase activity is assayed using luciferase lysis buffer and substrate (Promega) and measured using a luminometer (Turner Designs).

### 6.2. EXAMPLE 2

### Preparation of 3-(5-amino-2-methyl-4-oxo-4H-quinazolin-3-yl)-piperidine-2,6-dione (Compound 1)

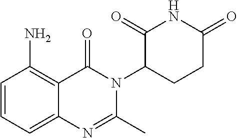 [SMILES: Cc1nc2cccc(N)c2c(=O)n1C1CCC(=O)NC1=O]

Step 1: To a solution of potassium hydroxide (16.1 g, 286 mmol) in water (500 mL), was added 3-nitrophthalimide (25.0 g, 130 mmol) in portion at 0° C. The suspension was stirred at 0° C. for 3 hrs, and then heated to 30° C. for 3 hrs. To the solution, was added HCl (100 mL, 6N). The resulting suspension was cooled to 0° C. for 1 hr. The suspension was filtered and washed with cold water (2×10 mL) to give 3-nitro-phthalamic acid as a white solid (24.6 g, 90% yield): ¹H NMR (DMSO-d₆) δ 7.69 (brs, 1H, NHH), 7.74 (t, J=8 Hz, 1H, Ar), 7.92 (dd, J=1, 8 Hz, 1H, Ar), 8.13 (dd, J=1, 8 Hz, 1H, Ar), 8.15 (brs, 1H, NHH), 13.59 (s, 1H, OH); ¹³C NMR (DMSO-d₆) δ 125.33, 129.15, 130.25, 132.54, 136.72, 147.03, 165.90, 167.31.

Step 2: To a mixture of 3-nitro-phthalamic acid (24.6 g, 117 mmol) and potassium hydroxide (6.56 g, 117 mmol) in water (118 mL), was added a mixture of bromine (6 mL), potassium hydroxide (13.2 g, 234 mmol) in water (240 mL) at 0° C., followed by addition of a solution of potassium hydroxide (19.8 g, 351 mmol) in water (350 mL). After 5 minutes at 0° C., the mixture was heated in a 100° C. oil bath for 1 hr. The reaction solution was cooled to room temperature, and then, in an ice-water bath for 30 minutes. To the mixture, a solution of HCl (240 mL, 2N) was added dropwise at 0° C., and the resulting mixture was kept for 1 hr. The supsension was filtered and washed with water (5 mL) to give 2-amino-6-nitro-benzoic acid as yellow solid (15.6 g, 73% yield): HPLC: Waters Symmetry C₁₈, 5 μm, 3.9×150 mm, 1 mL/min, 240 nm, CH₃CN/0.1% H₃PO₄, 5% grad to 95% over 5 min, 5.83 min (85%); ¹H NMR (DMSO-d₆) δ 6.90 (dd, J=1, 8 Hz, 1H, Ar), 7.01 (dd, J=1, 9 Hz, 1H, Ar), 7.31 (t, J=8 Hz, 1H, Ar), 8.5-9.5 (brs, 3H, OH, NH₂); ¹³C NMR (DMSO-d₆) δ 105.58, 110.14, 120.07, 131.74, 149.80, 151.36, 166.30; LCMS: MH=183.

Step 3: A mixture of 2-amino-6-nitro-benzoic acid (1.5 g, 8.2 mmol) in acetic anhydride (15 mL) was heated at 200° C. for 30 minutes in a microwave oven. The mixture was filtered and washed with ethyl acetate (20 mL). The filtrate was concentrated in vacuo. The solid was stirred in ether (20 mL) for 2 hrs. The suspension was filtered and washed with ether (20 mL) to give 2-methyl-5-nitro-benzo[d][1,3]oxazin-4-one as a light brown solid (1.4 g, 85% yield): HPLC: Waters Symmetry C₁₈, 5 μm, 3.9×150 mm, 1 mL/min, 240 nm, CH₃CN/0.1% H₃PO₄, 5% grad 95% in 5 min, 5.36 min (92%); ¹H NMR (DMSO-d₆) δ 2.42 (s, 3H, CH₃), 7.79 (dd, J=1, 8 Hz, 1H, Ar), 7.93 (dd, J=1, 8 Hz, 1H, Ar), 8.06 (t, J=8 Hz, 1H, Ar); ¹³C NMR (DMSO-d₆) δ 20.87, 107.79, 121.54, 128.87, 137.19, 147.12, 148.46, 155.18, 161.78; LCMS: MH=207.

Step 4: Two vials each with a suspension of 5-nitro-2-methyl-benzo[d][1,3]oxazin-4-one (0.60 g, 2.91 mmol) and 3-amino-piperidine-2,6-dione hydrogen chloride (0.48 g, 2.91 mmol) in pyridine (15 mL) were heated at 170° C. for 10 minutes in a microwave oven. The suspension was filtered and washed with pyridine (5 mL). The filtrate was concentrated in vacuo. The resulting mixture was stirred in HCl (30 mL, 1N), ethyl acetate (15 mL) and ether (15 mL) for 2 hrs. The suspension was filtered and washed with water (30 mL) and ethyl acetate (30 mL) to give a dark brown solid, which was stirred with methanol (50 mL) at room temperature overnight. The suspension was filtered and washed with methanol to give 3-(2-methyl-5-nitro-4-oxo-4H-quinazolin-3-yl)-piperidine-2,6-dione as a black solid (490 mg, 27% yield). The solid was used in the next step without further purification.

Step 5: A mixture of 3-(2-methyl-5-nitro-4-oxo-4H-quinazolin-3-yl)-piperidine-2,6-dione (250 mg) and Pd(OH)₂ on carbon (110 mg) in DMF (40 mL) was shaken under hydrogen (50 psi) for 12 hrs. The suspension was filtered through a pad of Celite and washed with DMF (10 mL). The filtrate was concentrated in vacuo and the resulting oil was purified by flash column chromatography (silica gel, methanol/methylene chloride) to give 3-(5-amino-2-methyl-4-oxo-4H-quinazolin-3-yl)-piperidine-2,6-dione as a white solid (156 mg, 69% yield): HPLC: Waters Symmetry C₁₈, 5 μm, 3.9×150 mm, 1 mL/min, 240 nm, 10/90 CH₃CN/0.1% H₃PO₄, 3.52 min (99.9%); mp: 293-295° C.; ¹H NMR (DMSO-d₆) δ 2.10-2.17 (m, 1H, CHH), 2.53 (s, 3H, CH₃), 2.59-2.69 (m, 2H, CH₂), 2.76-2.89 (m, 1H, CHH), 5.14 (dd, J=6, 11 Hz, 1H, NCH), 6.56 (d, J=8 Hz, 1H, Ar), 6.59 (d, J=8 Hz, 1H, Ar), 7.02 (s, 2H, NH₂), 7.36 (t, J=8 Hz, 1H, Ar), 10.98 (s, 1H, NH); ¹³C NMR (DMSO-d₆) δ 20.98, 23.14, 30.52, 55.92, 104.15, 110.48, 111.37, 134.92, 148.17, 150.55, 153.62, 162.59, 169.65, 172.57; LCMS: MH=287; Anal. Calcd. for C₁₄H₁₄N₄O₃+0.3 H₂O: C, 57.65; H, 5.05; N, 19.21. Found: C, 57.50; H, 4.73; N, 19.00.

### 6.3. EXAMPLE 3

### Salt and Cocrystal Screen of Compound 1

The salt and cocrystal screen was carried out using three experimental methodologies, as described below.

Experimental Methodology

Stoichiometric Precipitation and Cooling Experiments

Stoichiometric precipitation and cooling experiments were carried out in glass vials. About 20 mg of Compound land an approximately equimolar amount of coformer were dissolved in a given solvent. The solutions were combined. If precipitation occurred, the slurry was allowed to stir at ambient temperature for 2 days. If no precipitation occurred, the solution placed in a freezer to induce crystallization. The solids were isolated by filtration and analyzed by XRPD and other techniques as described below.

Stoichiometric Slurry Experiments

Stoichiometric slurry experiments were carried out in glass vials. Each of the vials was charged with about 20 mg of Compound 1, an approximately equimolar amount of coformer, and approximately 500 μL of a saturated solution of both Compound 1 and the same coformer in the solvent used for that experiment. A magnetic stir bar was placed in each vial and the rack of vials was placed on a stir plate at room temperature for 2 days. The solids were were isolated and analyzed by XRPD and other techniques as described below.

Stoichiometric Evaporation Experiments

Stoichiometric slow evaporation experiments were carried out in glass vials. Each of the vials was charged with about 20 mg of Compound 1 and an approximately equimolar amount of coformer. The contents were dissolved in a given solvent and placed in glass vials. The solutions were allowed to evaporate at ambient temperature. The solids were analyzed by XRPD and other techniques as described below.

Generation of Salts and/or Cocrystals

Compound 1 was mixed with various acids under various conditions in attempts to generate crystalline salts and cocrystals. The acids used are as follows: acetic acid, maleic acid, sulfuric acid, benzenesulfonic acid, methanesulfonic acid, thiocyanic acid, ethanesulfonic acid, naphthalene-1,5-disulfonic acid, 4-toluenesulfonic acid, isethionic acid, naphthalene-2-sulfonic acid, lauric acid and phosphoric acid.

The screening experiments are shown in Table 1.

TABLE 1

| Acid | Conditions |
|---|---|
| Acetic acid | SS, acetone |
| Benzenesulfonic acid | P, acetone |
| Ethanesulfonic acid | P, acetone; SL, acetone |
| Isethionic acid | E, acetone; SL, acetone |
| Lauric acid | SS, acetone |
| Maleic acid | E, ACN |
| Methanesulfonic acid | E, acetone |
| Naphthalene-1,5-disulfonic | E, ACN |
| acid | |
| Naphthalene-2-sulfonic acid | P, acetone |
| Phosphoric acid | P, acetone |
| Sulfuric acid | P, acetone |
| Thiocyanic acid | P, acetone; E, acetone |
| 4-Toluenesulfonic acid | P, acetone |

ACN = acetonitrile,
E = evaporation,
MeOH = methanol,
P = precipitation,
RT = room temperature,
SL = slurry,
SS = stoichiometric slurry.

Characterization Methodology

### X-Ray Powder Diffraction (XRPD)

The Rigaku Smart-Lab X-ray diffraction system was configured for reflection Bragg-Brentano geometry using a line source X-ray beam. The x-ray source is a Cu Long Fine Focus tube that was operated at 40 kV and 44 mA. That source provides an incident beam profile at the sample that changes from a narrow line at high angles to a broad rectangle at low angles. Beam conditioning slits are used on the line X-ray source to ensure that the maximum beam size is less than 10 mm both along the line and normal to the line. The Bragg-Brentano geometry is a para-focusing geometry controlled by passive divergence and receiving slits with the sample itself acting as the focusing component for the optics. The inherent resolution of Bragg-Brentano geometry is governed in part by the diffractometer radius and the width of the receiving slit used. Typically, the Rigaku Smart-Lab is operated to give peak widths of 0.1° 2θ or less. The axial divergence of the X-ray beam is controlled by 5.0° Soller slits in both the incident and diffracted beam paths.

Powder samples were prepared in a low background Si holder using light manual pressure to keep the sample surfaces flat and level with the reference surface of the sample holder. Each sample was analyzed from 2 to 40° 2θ using a continuous scan of 6° 2θ per minute with an effective step size of 0.02° 2θ.

### Differential Scanning Calorimetry (DSC):

DSC analyses were performed on a TA Instruments Q2000 differential scanning calorimeter. Indium was used as a calibration standard for instrument temperature. The DSC cell was kept under a nitrogen purge of ˜50 mL per minute during each analysis. The sample was placed in a standard, crimped, aluminum pan and was heated from approximately 25° C. to 350° C. at a rate of 10° C. per minute.

### Thermal Gravimetric Analyses (TGA):

TGA analyses were performed on a TA Instruments Q50 thermogravimetric analyzer. The instrument balance was calibrated using class M weights and the temperature calibration was performed using alumel. The nitrogen purge was ˜40 mL per minute at the balance and ˜60 mL per minute at the furnace. Each sample was placed into a pretared platinum pan and heated from approximately 25° C. to 350° C. at a rate of 10° C. per minute.

### Dynamic Vapor Sorption (DVS):

DVS analysis was carried out using a TA Instruments Q5000 Dynamic Vapor Sorption analyzer. The instrument was calibrated with standard weights and a sodium bromide standard for humidity. Approximately 10-25 mg of sample was loaded into a metalcoated quartz pan for analysis. The sample was analyzed at 25° C. with a maximum equilibration time of one hour in 10% relative humidity (RH) steps from 5 to 95% RH (adsorption cycle) and from 95 to 5% RH (desorption cycle). The movement from one step to the next occurred either after satisfying the equilibrium criterion of 0.01% weight change or, if the equilibrium criterion was not met, after one hour. The percent weight change values were calculated using Microsoft Excel®.

### Nuclear Magnetic Resonance (NMR) Spectroscopy

The 1H NMR spectra were acquired on a Bruker DRX-500 spectrometer located at the Chemistry Department of Purdue University. Samples were prepared by dissolving material in DMSO-d₆. The solutions were filtered and placed into individual 5-mm NMR tubes for subsequent spectral acquisition. The temperature controlled (298K) 1H NMR spectra acquired on the DRX-500 utilized a 5-mm cryoprobe operating at an observing frequency of 499.89 MHz.

### Solid Form Screening Study Results

Solid forms comprising Compound 1 which were prepared during the solid form screening studies included Form A of a besylate salt of Compound 1, Form A or Form B of an esylate salt of Compound 1, Form A of a sulfate salt of Compound 1, Form A or Form B of a mesylate salt of Compound 1, Form A of a napadisylate salt of Compound 1, Form A of a napsilate salt of Compound 1, Form A or Form B of a thiocyanate salt of Compound 1, and Form A or Form B of a tosylate salt of Compound 1; Compound 1 acetic acid cocrystal Form A, Compound 1 isethionic acid cocrystal Form A, Compound 1 isethionic acid cocrystal Form B, Compound 1 lauric acid cocrystal Form A, Compound 1 maleic acid cocrystal Form A, Compound 1 phosphoric acid cocrystal Form A.

Representative XRPD patterns, DSC plots, TGA plots and DVS plots and NMR spectra for Form A of a besylate salt of Compound 1, Form A or Form B of an esylate salt of Compound 1, Form A of a sulfate salt of Compound 1, Form A or Form B of a mesylate salt of Compound 1, Form A of a napadisylate salt of Compound 1, Form A of a napsilate salt of Compound 1, Form A or Form B of a thiocyanate salt of Compound 1, and Form A or Form B of a tosylate salt of Compound 1; Compound 1 acetic acid cocrystal Form A, Compound 1 isethionic acid cocrystal Form A, Compound 1 isethionic acid cocrystal Form B, Compound 1 lauric acid cocrystal Form A, Compound 1 maleic acid cocrystal Form A, Compound 1 phosphoric acid cocrystal Form A are provided herein as FIGS. 1 to 51.

### Solid Forms of Compound 1

### Besylate Salt of Compound 1

One polymorph of the besylate salt has been observed todate, and was designated as Form A. The characterization data for Compound 1 besylate salt Form A are shown in Table 2. Compound 1 besylate salt Form A is an unsolvated form and has stoichiometry of 1:1. Thermogravimetry data showed 0.002% weight loss below 225° C. which is likely due to loss of residual solvent. Approximately 0.04 moles of acetone was observed in the NMR spectrum. Compound 1 besylate salt Form A was moderately hygroscopic.

TABLE 2

| Technique | FIG. No. | Result |
|---|---|---|
| XRPD | FIG. 1A | Besylate Form A |
| DSC | FIG. 2 | endo 278.79° C. |
| TG | FIG. 2 | 0.002% up to 225° C. |
| DVS | FIG. 3 | 0.16% loss upon drying at 5% RH |
| | | 4.31% gain from 5 to 95% RH |
| | | 4.31% loss from 95 to 5% RH |
| Post-DVS XRPD | FIG. 1B | Unchanged |
| NMR | FIGS. 4A, 4B and | 1:1 salt |
| | 4C | |

### Esylate Salt of Compound 1

Two polymorphs of the esylate salt has been observed todate, and were designated as Form A and Form B. Representative XRPD patterns of esylate salt Form A and Form B Compound 1 are overlaid and shown in FIG. 6.

The characterization data are shown in Table 3. Compound 1 esylate salt Form B is an unsolvated form and has stoichiometry of 1:1. Thermogravimetry data showed 2.0% weight loss below 50° C. which is likely due to loss of residual solvent. Approximately 0.06 moles of acetone was observed in the NMR spectrum.

TABLE 3

| Technique | FIG. No. | Result |
|---|---|---|
| XRPD | FIG. 5 | Esylate Form B |
| DSC | FIG. 7 | endo 76.98, 95.44, 228.15° C. |
| TG | FIG. 7 | 2.03% up to 50° C. |
| | | 0.49% from 50 to 200° C. |
| NMR | FIGS. 8A and 8B | 1:1 salt |

### Napadisylate Salt of Compound 1

One polymorph of the napadisylate salt has been observed todate, and was designated as Form A. The characterization data are shown in Table 4. Compound 1 napadisylate salt Form A is an unsolvated form and has stoichiometry of 1:1. Thermogravimetry data showed 1.52% weight loss below 275° C. which is likely due to loss of residual solvent. Approximately 0.15 moles of acetone was observed in the NMR spectrum.

TABLE 4

| Technique | FIG. No. | Result |
|---|---|---|
| XRPD | FIG. 9 | Napadisylate Form A |
| DSC | FIG. 10 | endo 315.91° C. |
| TG | FIG. 10 | 1.52% up to 275° C. |
| NMR | FIGS. 11A and 11B | 1:1 salt |

### Napsilate Salt of Compound 1

One polymorph of the napsilate salt has been observed todate, and was designated as Form A. The characterization data are shown in Table 5. Compound 1 napsilate salt Form A is an unsolvated form and has stoichiometry of 1:1. The thermogravimetry data showed 3.36% weight loss below 100° C. which is likely due to loss of residual solvent. Approximately 0.09 moles of acetone was observed in the NMR spectrum.

TABLE 5

| Technique | FIG. No. | Result |
|---|---|---|
| XRPD | FIG. 12 | Napsilate Form A |
| DSC | FIG. 13 | endo 109.30 and 282.55° C. |
| TG | FIG. 13 | 3.36% up to 100° C. |
| | | 0.15% from 100 to 225° C. |
| NMR | FIGS. 13A, 13B and 13C | 1:1 salt |

### Mesylate Salt of Compound 1

Two polymorph forms of the mesylate salt have been observed todate, and were designated as Form A and Form B. Representative XRPD patterns of Compound 1 mesylate salt Form A and Form B are overlaid and shown in FIG. 16.

The characterization data for mesylate salt forms are shown in Table 6. Compound 1 mesylate salt Form A is an unsolvated form and has stoichiometry of 1:1. Thermogravimetry data showed 2.84% weight loss below 225° C. Approximately 0.10 moles of acetone was observed in the NMR spectrum. Compound 1 mesylate salt Form A is moderately hygroscopic.

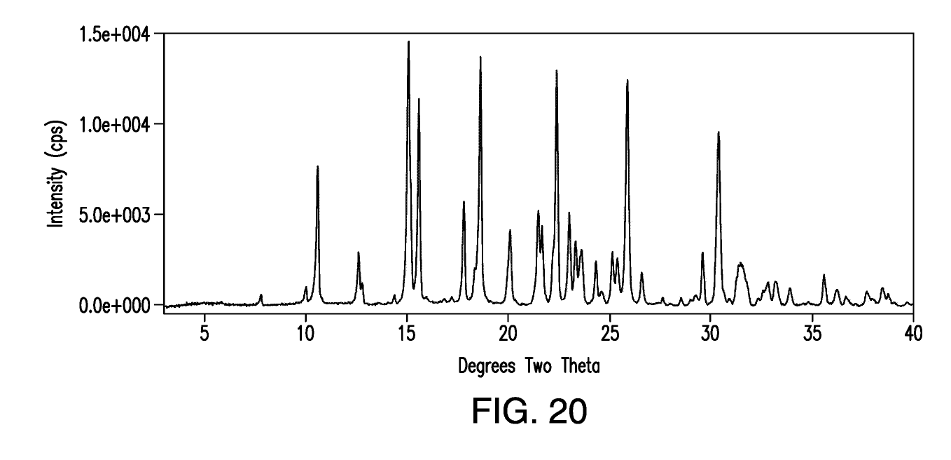

TABLE 6

| Technique | FIG. No. | Result |
|---|---|---|
| XRPD | FIG. 15A | Mesylate Form A |
| DSC | FIG. 17 | endo 137.16, 282.88° C. |
| TG | FIG. 17 | 2.84% up to 225° C. |
| DVS | FIG. 19 | 0.17% loss upon drying at 5% RH |
| | | 9.79% gain from 5 to 95% RH |
| | | 7.98% loss from 95 to 5% RH |
| Post-DVS XRPD | FIG. 15B | Mesylate Form B |
| NMR | FIGS. 20A and | 1:1 salt |
| | 20B | |

### Sulfate Salt of Compound 1

One polymorph form of the sulfate salt has been observed todate, and was designated as Form A. The characterization data for the sulfate salt form are shown in Table 7. Compound 1 sulfate salt Form A is an unsolvated form and has stoichiometry of 1:1. The thermogravimetry data showed 0.96% weight loss below 240° C. Approximately 0.10 moles of acetone was observed in the NMR spectrum. Compound 1 sulfate salt Form A is moderately hygroscopic.

TABLE 7

| Technique | FIG. No. | Result | | | | |
|---|---|---|---|---|---|---|
| XRPD | FIG. 21 | Sulfate Form A | | | | |
| DSC | FIG. 22 | endo 247.32, 274.37° C. | | | | |
| TG | FIG. 22 | 0.96% up to 240° C. | | | | |
| DVS | FIG. 23 | 0.21% loss on drying at 5% RH | | | | |
| | | 5.36% gain from 5 to 95% RH | | | | |
| | | 5.63% loss from 95 to 5% RH | | | | |
| Post-DVS XRPD | FIG. 24 | Unchanged | | | | |
| NMR | FIGS. 25A | — | | | | |
| | and 25B | | | | | |
| | | — | % C | % H | % N | % S |
| Elemental Analysis | — | Theo | 43.74 | 4.20 | 14.58 | 8.34 |
| | | (1:1) | | | | |
| | | Result | 43.40 | 4.03 | 14.15 | 7.90 |

### Thiocyanate Salt of Compound 1

Two polymorph forms of the thiocyanate salt have been observed todate, and were designated as Form A and Form B. Representative XRPD patterns of Compound 1 thiocyanate salt Form A and Form B are overlaid and shown in FIG. 28.

The characterization data for the thiocyanate salt form are shown in Table 8. Compound 1 thiocyanate salt Form A is an unsolvated form and has stoichiometry of 1:1. The thermogravimetry data showed 0.62% weight loss below 125° C. The weight loss of 17.25% from 125 to 175° C. corresponds to volatilization of approximately one mole of thiocyanic acid. Approximately 0.20 moles of acetone was observed in the NMR spectrum.

TABLE 8

| Technique | FIG. No. | Result | | | | |
|---|---|---|---|---|---|---|
| XRPD | FIG. 26 | Thiocyanate Form A | | | | |
| DSC | FIG. 27 | endo 167.18, 186.42, 228.08° C. | | | | |
| TG | FIG. 27 | 0.62% loss up to 125° C. | | | | |
| | | 17.25% loss from 125 to 225° C. | | | | |
| NMR | FIGS. 29A and 29B | — | | | | |
| | | — | % C | % H | % N | % S |
| Elemental | — | Theo (1:1) | 52.16 | 4.38 | 20.28 | 9.28 |
| Analysis | | Result | 52.24 | 4.57 | 18.73 | 8.44 |

### Tosylate Salt of Compound 1

Two polymorph forms of the tosylate salt have been observed todate, and were designated as Form A and Form B. Representative XRPD patterns of Compound 1 tosylate salt Form A and Form B are overlaid and shown in FIG. 34.

The characterization data for the tosylate salt forms are shown in Table 9. Compound 1 tosylate salt Form A is an unsolvated form and has stoichiometry of 1:1. The thermogravimetry data showed 0.51% weight loss below 250° C. Approximately 0.08 moles of acetone was observed in the NMR spectrum.

TABLE 9

| Technique | FIG. No. | Result |
|---|---|---|
| XRPd | FIG. 30 | Tosylate Form A |
| DSC | FIG. 31 | endo 285.32° C. |
| TG | FIG. 31 | 0.51% up to 250° C. |
| DVS | FIG. 32 | 0.14% loss upon drying at 5% |
| | | RH |
| | | 1.15% gain from 5 to 95% RH |
| | | 1.17% loss from 95 to 5% RH |
| Post-DVS XRPD | FIG. 33 | Tosylate form B |
| NMR | FIGS. 35A and 35B | 1:1 salt |

### Compound 1 Acetic Acid Cocrystal

One polymorph of the acetic acid cocrystal has been observed todate, and was designated as Form A. The characterization data are shown in Table 10. Acetic acid cocrystal Form A of Compound 1 is an unsolvated form and has stoichiometry of 1:1. The thermogravimetry data showed 21.90% weight loss below 140° C. which corresponds to 1.3 moles of acetic acid. The similar amount of acetic acid was observed in the NMR spectrum. There are two steps in the TG curve, the first ending around 80° C. (volatilization of residual acetic acid) and the second ending around 140° C. (breaking of cocrystal and volatilization of acetic acid).

TABLE 10

| Technique | FIG. No. | Result |
|---|---|---|
| XRPD | FIG. 36 | acetic acid cocrystal Form A |
| DSC | FIG. 37 | endo 109.08 and 277.56° C. |
| TG | FIG. 37 | 21.90% loss up to 140° C. |
| NMR | FIGS. 38A and 38B | 1:1 cocrystal |

### Compound 1 Isethionic Acid Cocrystal

Two polymorphs of the isethionic acid cocrystal has been observed todate, and were designated as Form A and Form B. Representative XRPD patterns of Compound 1 isethionic cocrystal Form A and Form B are overlaid and shown in FIG. 40.

The characterization data are shown in Table 11. Isethionic acid cocrystal Form B of Compound 1 is solvated form and has stoichiometry of 1:1. The thermogravimetry data showed about 4.0% weight loss below 165° C. which corresponds to 0.21 moles of acetone. The TG weight loss occurs at a high enough temperature to suggest that the solvent is not residual.

TABLE 11

| Technique | FIG. No. | Result |
|---|---|---|
| XRPD | FIG. 39 | isethionic acid cocrystal Form B |
| DSC | FIG. 41 | endo 147.00, 186.11° C. |
| TG | FIG. 41 | 3.98% loss up to 165° C. |
| NMR | FIGS. 42A and 42B | 1:1 cocrystal |

### Compound 1 Lauric Acid Cocrystal

One polymorph of the lauric acid cocrystal has been observed todate, and was designated as Form A. The characterization data are shown in Table 12. Laurie acid cocrystal of Compound 1 is an unsolvated form and has stoichiometry of 1:1. The thermogravimetry data showed 0.14% weight loss below 90° C. The weight loss of 40.45% from 90 to 145° C. corresponds to approximately 1 mole of lauric acid. The endotherm at 45.05° C. in the DSC curve is likely due to lauric acid melting.

TABLE 12

| Technique | FIG. No. | Result |
|---|---|---|
| XRPD | FIG. 43 | lauric acid cocrystal Form A |
| DSC | FIG. 44 | endo 45.05° C. |
| TG | FIG. 44 | 0.14% up to 90° C. |
| | | 40.45% from 90 to 175° C. |
| NMR | FIGS. 45A and 45B | 1:1 cocrystal |

### Compound 1 Maleic Acid Cocrystal

One polymorph of the maleic acid cocrystal has been observed todate, and was designated as Form A. The characterization data are shown in Table 13. Maleic acid cocrystal Form A of Compound 1 is a solvated form and has stoichiometry of 2:1 (Compound 1 to maleic acid). The thermogravimetry data showed about 6.8% weight loss below 125° C. which corresponds to 0.5 moles of acetonitrile. Approximately 0.5 moles of acetonitrile was observed in the NMR spectrum.

TABLE 13

| Technique | FIG. No. | Result |
|---|---|---|
| XRPD | FIG. 46 | maleic acid cocrystal Form A |
| DSC | FIG. 47 | endo 106.50° C. |
| TG | FIG. 47 | 6.78% up to 125° C. |
| NMR | FIGS. 48A and 48B | 2:1 cocrystal |

### Compound 1 Phosphoric Acid Cocrystal

One polymorph of the phosphoric acid cocrystal has been observed todate, and was designated as Form A. The characterization data are shown in Table 14. Phosphoric acid cocrystal Form A of Compound 1 is a solvated form and has stoichiometry of 2:3 (Compound 1 to phosphoric acid). The thermogravimetry data showed 4.14% weight loss below 175° C. Approximately 0.27 moles of acetone was observed in the NMR spectrum.

TABLE 14

| Technique | FIG. No. | Result | | | | |
|---|---|---|---|---|---|---|
| XRPD | FIG. 49 | phosphoric acid cocrystal Form A | | | | |
| DSC | FIG. 50 | endo 116.89, 166.37, 222.27° C. | | | | |
| TG | FIG. 50 | 4.14% up to 175° C. | | | | |
| NMR | FIGS. 51A and 51B | — | | | | |
| | | — | % C | % H | % N | % S |
| Elemental | — | Theo (1:1) | 43.75 | 4.46 | 14.58 | 8.06 |
| Analysis | | Theo (2:3) | 38.81 | 4.30 | 12.93 | 10.72 |
| | | Result | 38.15 | 4.51 | 12.11 | 10.00 |

While the disclosure has been described with respect to the particular embodiments, it will be apparent to those skilled in the art that various changes and modifications may be made without departing from the spirit and scope of the disclosure as defined in the claims. Such modifications are also intended to fall within the scope of the appended claims.

All of the patents, patent applications and publications referred to herein are incorporated herein in their entireties. Citation or identification of any reference in this application is not an admission that such reference is available as prior art to this disclosure. The full scope of the disclosure is better understood with reference to the appended claims.

What is claimed is:

1. A solid form compring a salt or a cocrystal of 3-(5-amino-2-methyl-4-oxo-4H-quinazolin-3-yl)-piperidine-2,6-dione: 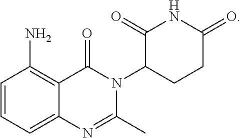 [SMILES: Cc1nc2cccc(N)c2c(=O)n1C1CCC(=O)NC1=O]
wherein the salt or cocrystal is selected from a besylate salt, an esylate salt, a napadisylate salt, a napsilate salt, a mesylate salt, a sulfate salt, a thiocyanate salt, a tosylate salt, an acetic acid cocrystal, an isethionic acid cocrystal, a lauric acid cocrystal, a maleic acid cocrystal, and a phosphoric acid cocrystal.

2. The solid form of claim 1, which is Form A of a besylate salt of 3-(5-amino-2-methyl-4-oxo-4H-quinazolin-3-yl)-piperidine-2,6-dione.

3. The solid form of claim 1, having an X-ray powder diffraction pattern substantially as shown in FIG. 1.

4. The solid form of claim 1, having a differential scanning calorimetry plot comprising an endothermic event with a peak temperature of about 279° C.

5. The solid form of claim 1, having a thermal gravimetric analysis plot comprising a weight loss of about 0.002% when heated up to about 225° C.

6. The solid form of claim 1, which exhibits a weight increase of about 4.31% when subjected to an increase in relative humidity from about 5% to about 95%.

7. The solid form of claim 1, which is Form B of an esylate salt of 3-(5-amino-2-methyl-4-oxo-4H-quinazolin-3-yl)-piperidine-2,6-dione.

8. The solid form of claim 1, having an X-ray powder diffraction pattern substantially as shown in FIG. 5.

9. The solid form of claim 7, having a differential scanning calorimetry plot comprising endothermic events with peak temperatures of about 77, 95 or 228° C.

10. The solid form of claim 7, having a thermal gravimetric analysis plot comprising a weight loss of about 2.0% when heated up to about 50° C.

11. The solid form of claim 1, which is Form A of a napadisylate salt of 3-(5-amino-2-methyl-4-oxo-4H-quinazolin-3-yl)-piperidine-2,6-dione.

12. The solid form of claim 1, having an X-ray powder diffraction pattern substantially as shown in FIG. 9.

13. The solid form of claim 11, having a differential scanning calorimetry plot comprising an endothermic event with a peak temperature of about 316° C.

14. The solid form of 11, having a thermal gravimetric analysis plot comprising a weight loss of about 1.52% when heated up to about 275° C.

15. The solid form of claim 1, which is Form A of a napsilate salt of 3-(5-amino-2-methyl-4-oxo-4H-quinazolin-3-yl)-piperidine-2,6-dione.

16. The solid form of claim 1, having an X-ray powder diffraction pattern substantially as shown in FIG. 12.

17. The solid form of claim 15, having a differential scanning calorimetry plot comprising endothermic events with peak temperatures of about 109 or 283° C.

18. The solid form of claim 15 having a thermal gravimetric analysis plot comprising a weight loss of about 3.36% when heated up to about 100° C.

19. The solid form of claim 1, which is Form A of a mesylate salt of 3-(5-amino-2-methyl-4-oxo-4H-quinazolin-3-yl)-piperidine-2,6-dione.

20. The solid form of claim 1, having an X-ray powder diffraction pattern substantially as shown in FIG. 15.

21. The solid form of claim 19, having a differential scanning calorimetry plot comprising endothermic events with peak temperatures of about 137 or 283° C.

22. The solid form of claim 19, having a thermal gravimetric analysis plot comprising a weight loss of about 2.84% when heated up to about 225° C.

23. The solid form of claim 1, which is Form B of a mesylate salt of 3-(5-amino-2-methyl-4-oxo-4H-quinazolin-3-yl)-piperidine-2,6-dione.

24. The solid form of claim 23, having an X-ray powder diffraction pattern substantially as shown in FIG. 16.

25. The solid form of claim 1, which is Form A of a sulfate salt of 3-(5-amino-2-methyl-4-oxo-4H-quinazolin-3-yl)-piperidine-2,6-dione.

26. The solid form of claim 1, having an X-ray powder diffraction pattern substantially as shown in FIG. 21.

27. The solid form of claim 25, having a differential scanning calorimetry plot comprising endothermic events with peak temperatures of about 247 or 274° C.

28. The solid form of claim 25, having a thermal gravimetric analysis plot comprising a weight loss of about 0.96% when heated up to about 240° C.

29. The solid form of claim 25, which exhibits a weight increase of about 5.36% when subjected to an increase in relative humidity from about 5% to about 95%.

30. The solid form of claim 1, which is Form A of a thiocyanate salt of 3-(5-amino-2-methyl-4-oxo-4H-quinazolin-3-yl)-piperidine-2,6-dione.

31. The solid form of claim 30, having an X-ray powder diffraction pattern substantially as shown in FIG. 26.

32. The solid form of claim 30, having a differential scanning calorimetry plot comprising endothermic events with peak temperatures of about 167, 186 or 228° C.

33. The solid form of claim 30, having a thermal gravimetric analysis plot comprising a weight loss of about 0.62% when heated up to about 125° C.

34. The solid form of claim 1, which is Form A of a tosylate salt of 3-(5-amino-2-methyl-4-oxo-4H-quinazolin-3-yl)-piperidine-2,6-dione.

35. The solid form of claim 34, having an X-ray powder diffraction pattern substantially as shown in FIG. 30.

36. The solid form of claim 34, having a differential scanning calorimetry plot comprising an endothermic event with a peak temperature of about 285° C.

37. The solid form of claim 34, having a thermal gravimetric analysis plot comprising a weight loss of about 0.51% when heated up to about 250° C.

38. The solid form of claim 34, which exhibits a weight increase of about 1.15% when subjected to an increase in relative humidity from about 5% to about 95%.

39. The solid form of claim 1, which is Form B of a tosylate salt of 3-(5-amino-2-methyl-4-oxo-4H-quinazolin-3-yl)-piperidine-2,6-dione.

40. The solid form of claim 39, having an X-ray powder diffraction pattern substantially as shown in FIG. 33.

41. The solid form of claim 1, which is 3-(5-amino-2-methyl-4-oxo-4H-quinazolin-3-yl)-piperidine-2,6-dione acetic acid cocrystal Form A.

42. The solid form of claim 41, having an X-ray powder diffraction pattern substantially as shown in FIG. 36.

43. The solid form of claim 41, having a differential scanning calorimetry plot comprising endothermic events with peak temperatures of about 109 or 278° C.

44. The solid form of claim 41, having a thermal gravimetric analysis plot comprising a weight loss of about 21.90% when heated up to about 140° C.

45. The solid form of claim 1, which is 3-(5-amino-2-methyl-4-oxo-4H-quinazolin-3-yl)-piperidine-2,6-dione isethionic acid cocrystal Form B.

46. The solid form of claim 45, having an X-ray powder diffraction pattern substantially as shown in FIG. 39.

47. The solid form of claim 45, having a differential scanning calorimetry plot comprising endothermic events with peak temperatures of about 147 or 186° C.

48. The solid form of claim 45, having a thermal gravimetric analysis plot comprising a weight loss of about 4.0% when heated up to about 165° C.

49. The solid form of claim 1, which is 3-(5-amino-2-methyl-4-oxo-4H-quinazolin-3-yl)-piperidine-2,6-dione lauric acid cocrystal Form A.

50. The solid form of claim 49, having an X-ray powder diffraction pattern substantially as shown in FIG. 43.

51. The solid form of claim 49, having a differential scanning calorimetry plot comprising an endothermic event with a peak temperature of about 45° C.

52. The solid form of claim 49, having a thermal gravimetric analysis plot comprising a weight loss of about 0.14% when heated up to about 90° C.

53. The solid form of claim 1, which is 3-(5-amino-2-methyl-4-oxo-4H-quinazolin-3-yl)-piperidine-2,6-dione maleic acid cocrystal Form A.

54. The solid form of claim 53, having an X-ray powder diffraction pattern substantially as shown in FIG. 46.

55. The solid form of claim 54 having a differential scanning calorimetry plot comprising an endothermic event with a peak temperature of about 107° C.

56. The solid form of claim 53, having a thermal gravimetric analysis plot comprising a weight loss of about 6.8% when heated up to about 125° C.

57. The solid form of claim 1, which is 3-(5-amino-2-methyl-4-oxo-4H-quinazolin-3-yl)-piperidine-2,6-dione phosphoric acid cocrystal Form A.

58. The solid form of claim 57, having an X-ray powder diffraction pattern substantially as shown in FIG. 49.

59. The solid form of claim 57, having a differential scanning calorimetry plot comprising endothermic events with peak temperatures of about 117, 166 or 222° C.

60. The solid form of claim 57, having a thermal gravimetric analysis plot comprising a weight loss of about 4.1% when heated up to about 175° C.

61. A mixture comprising two or more solid forms of claim 1.

62. A pharmaceutical composition comprising the solid form of claim 1 and a pharmaceutical acceptable carrier, diluent or excipient.

63. The pharmaceutical composition of claim 62, wherein the composition is formulated for oral, parenteral, or intravenous administration.

64. The pharmaceutical composition of claim 62, wherein the composition is formulated as a single unit dosage form.

65. The pharmaceutical composition of claim 64, wherein the oral dosage form is a tablet or capsule.

66. A method of treating, managing or preventing a disease or disorder comprising administering to a patient a solid form of claim 1, wherein the disease or disorder is cancer or a disorder associated with angiogenesis.

67. The method of claim 66, further comprising administering a second active agent.
